(19) United States
(12) Patent Application Publication
(10) Pub. No.: US 2022/0008368 A1
(43) Pub. Date: Jan. 13, 2022

(54) METHODS AND COMPOSITIONS RELATED TO TARGETING FFAR2 AND ILC3 POPULATIONS FOR THE TREATMENT OF A GASTROINTESTINAL DISEASE

(71) Applicant: PRESIDENT AND FELLOWS OF HARVARD COLLEGE, Cambridge, MA (US)

(72) Inventors: Eunyoung CHUN, Brookline, MA (US); Wendy Sarah GARRETT, Brookline, MA (US)

(73) Assignee: PRESIDENT AND FELLOWS OF HARVARD COLLEGE, Cambridge, MA (US)

(21) Appl. No.: 17/293,840

(22) PCT Filed: Nov. 14, 2019
(86) PCT No.: PCT/US2019/061358

§ 371 Date: May 13, 2021

Related U.S. Application Data
(60) Provisional application No. 62/767,847, filed on Nov. 15, 2018.

(51) Int. Cl. A61K 31/19; A61K 31/426; A61K 31/513; A61K 38/17; A61P 1/04; A61P 29/00; G01N 33/92; G01N 33/50

(52) U.S. Cl. CPC A61K 31/19; A61K 31/426; A61K 31/513; A61K 38/177; A61K 48/00; A61P 29/00; G01N 33/92; G01N 33/5047; A61P 1/04

(57) ABSTRACT

Described herein are methods, assays, and compositions and uses thereof related to treating, preventing, and detecting a gastrointestinal disease with an agent that targets Ffar2. The agents described herein can further increase populations of group 3 innate lymphoid cells (ILC3s) in the gut.

### CROSS-REFERENCE TO RELATED APPLICATION

This application claims benefit under 35 U.S.C. § 119(e) of U.S. Provisional Application No. 62/767,847 filed Nov. 15, 2018, the content of which is incorporated herein by reference in its entirety.

### GOVERNMENT SUPPORT

This invention was made with government support under Grant No. R01CA154426, awarded by the National Institutes of Health. The government has certain rights in the invention.

### TECHNICAL FIELD

The technology described herein relates to methods, compositions, and assays for treating, preventing, and detecting a gastrointestinal disease and uses thereof.

### BACKGROUND

Gastrointestinal diseases (e.g. ulcerative colitis) are debilitating diseases that result in abdominal pain, diarrhea, weight loss, fever, among other symptoms. The gut has group 3 innate lymphoid cells (ILC3s) that sense environmental signals useful for the normal activity in the body's defense from infections caused by microorganisms (e.g. bacteria). Short-chain fatty acids (SCFAs), are a type of microbial metabolite that have emerged as significant regulators of immune responses in the gut and systemically. Ffar2, a SCFA-sensing receptor, is broadly immunomodulatory and plays a significant role in regulation of inflammation within the gut. However, the metabolite-sensing receptors that regulate ILC3s in the gastrointestinal tract remain poorly understood. Thus, there is an unmet need for therapeutics that target ILC3 function to treat gastrointestinal diseases and infections.

### SUMMARY

Provided herein are methods, assays, and compositions related to treating and preventing a gastrointestinal disease in a subject.

In one aspect, described herein is method for treating or preventing a gastrointestinal disease, the method comprises: administering to a subject in need thereof an agent that increases the level or activity of Free Fatty Acid Receptor 2 (Ffar2) in the subject.

In another aspect, described herein is an assay for identifying an agent that modulates a functional property of an immune lymphoid cell, the assay comprises: (a) contacting a population of innate lymphoid cells with an agent; and (b) detecting the level of Ffar2; wherein detecting a change in Ffar2 levels after contacting step (a) identifies the agent as one that can modulate a functional property of innate lymphoid cells.

In another aspect, described herein is a method of treating a gastrointestinal disease in a subject, the method comprises: (a) measuring the level of Ffar2 in a biological sample of a subject; and (b) comparing the measurement of (a) to a reference level; (c) identifying a subject with decreased Ffar2 in (a) as compared to a reference level as having a gastrointestinal disease; and (d) administering to the subject having a gastrointestinal disease an agent that modulates Ffar2.

In another aspect, described herein is a method of reducing inflammation in the gastrointestinal tract of a subject, the method comprises: administering to a subject an agent that increases the level or activity of Free Fatty Acid Receptor 2 (Ffar2) in the subject.

In another aspect, described herein is a pharmaceutical composition formulated for the treatment of a gastrointestinal disease, the pharmaceutical composition comprising: an agent that increases the level or activity of Ffar2, wherein the pharmaceutical composition increases the number of group 3 innate lymphoid cells (ILC3s) in a subject.

In one embodiment of any of the aspects, the agent preferentially binds to a Ffar2 receptor.

In another embodiment of any of the aspects, the agent induces an increase in the number of group 3 innate lymphoid cells (ILC3s).

In another embodiment of any of the aspects, the agent induces secretion of interleukin-22 (IL-22) and/or interleukin-17 (IL-17) from ILC3s.

In another embodiment of any of the aspects, the agent is selected from the group consisting of a small molecule, an antibody, a peptide, a genome editing system, a vector, nucleic acid, a miRNA, and a siRNA.

In another embodiment of any of the aspects, the small molecule is a short chain fatty acid (SCFA) or derivative thereof.

In another embodiment of any of the aspects, the small molecule is selected from the group consisting of: ES43012-SOD, trans-2-methylcrotonic acid, propiolic acid, angelic acid, compound 34, sodium acetate, sodium propionate, sodium butyrate, formate, pentanoate, (S)-2-(4-chlorophenyl)-3,3-dimethyl-N-(5-phenylthiazol-2-yl)butanamide, BTI-A-404, BTI-A-292, AZ1729, and any derivative thereof.

In another embodiment of any of the aspects, the agent is a vector that encodes the agent. In another embodiment, the vector or nucleic acid encodes a Ffar2 polypeptide

In another embodiment of any of the aspects, the vector is non-integrative or integrative. In another embodiment of any of the aspects, the non-integrative vector is selected from the group consisting of an episomal vector, an EBNA1 vector, a minicircle vector, a non-integrative adenovirus, a non-integrative RNA, and a Sendai virus. In another embodiment of any of the aspects, the vector is an episomal vector. In another embodiment of any of the aspects, the vector is a lentiviral vector.

In another embodiment of any of the aspects, the agent is formulated with a pharmaceutical composition.

In another embodiment of any of the aspects, the pharmaceutical composition is formulated to restrict delivery of the agent to the gastrointestinal tract of the subject. In another embodiment, the pharmaceutical composition comprises an enteric coating.

In another embodiment of any of the aspects, the gastrointestinal disease is selected from the group consisting of: a gastrointestinal infection, inflammatory bowel disease (IBD), gastrointestinal injury, appendicitis, Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer.

In another embodiment of any of the aspects, the administering reduces inflammation of the gastrointestinal tract.

In another embodiment of any of the aspects, the subject is a mammal. In another embodiment of any of the aspects, the mammal is a human.

In another embodiment of any of the aspects, the level or activity of Ffar2 is increased by at least 50%, at least 60%, at least 70%, at least 80%, at least 90%, or more as compared to an appropriate control.

In another embodiment of any of the aspects, the secretion of IL-22 and/or IL-17 from ILC3s is increased by at least 50%, at least 60%, at least 70%, at least 80%, at least 90%, or more as compared to an appropriate control.

In another embodiment of any of the aspects, the agent is administered by direct injection, subcutaneous injection, muscular injection, oral, or nasal administration.

In another embodiment of any of the aspects, detecting step (b) further comprises detecting the level of IL-22. In another embodiment of any of the aspects, detecting step (b) further comprises detecting the level of RORγt, X-box binding protein-1 (XBP1), phosphorylated-Akt, phosphorylated STAT3, phosphorylated ERK, mucin 2, mucin 3, mucin 4, mucin 5a, mucin 5b, Regenerating islet-derived protein (Reg) 3 alpha, Reg 3 beta, Reg 3 gamma, and/or Ki-67.

In another embodiment of any of the aspects, the innate lymphoid cells are group 3 innate lymphoid cells (ILC3).

In another embodiment of any of the aspects, the method further comprises, prior to (a), obtaining a biological sample from the subject.

In another embodiment of any of the aspects, the biological sample is a blood sample, buffy coat, serum, or tissue. In another embodiment of any of the aspects, the tissue is removed from the esophagus, small intestine, large intestine, or colon.

### BRIEF DESCRIPTION OF THE DRAWINGS

FIG. 1A-1D shows that Ffar2 agonism selectively promotes colonic RORγt+ ILC3 expansion. FIG. 1A shows Ffar2 mRNA expression in the indicated cell population from the colon of WT mice (n=6 mice pooled per cell type per experiment). mRNA expression was normalized to housekeeping gene Actb. NK, conventional NK cells (NK1.1+); G, granulocytes (CD11b+Gr-1+); Mac, macrophages (CD11b+Gr-1−CD11c−); DC, conventional dendritic cells (CD11c+MHCII+), and ILC, innate lymphoid cells (Lin−CD90.2+). FIG. 1B shows Ffar2 mRNA expression in colonic ILC subsets from WT mice (n=6 mice pooled per cell subsets per experiment). FIG. 1C shows flow cytometry analysis of colonic ILC populations from WT mice fed with an Ffar2 agonist (n=9) or control (n=6). Colonic lamina propria (LP) cells were isolated and stained with viability dye, antibodies to lineage markers (CD3, Gr-1, CD11b, CD45R/B220, and Ter-119), anti-NK1.1, anti-CD45, anti-CD90.2, anti-Gata3, and anti-RORγt. Percentage among ILCs and the number of ILC1s (gated on live CD45+Lin−NK1.1−CD90.2+GATA3− RORγt−), ILC2s (gated on live CD45+Lin−NK1.1−CD90.2+GATA3+ RORγt−) and ILC3s (gated on live CD45+Lin−NK1.1−CD90.2+GATA3− RORγt+) were analyzed. FIG. 1D shows Ki-67 expression in colonic ILC2s and ILC3s from WT fed with the Ffar2 agonist (n=7) or control (n=6). Flow cytometry plots of live ILC2s (gated on live CD45+Lin−NK1.1−CD90.2+GATA3+ RORγt−) and ILC3s (gated on live CD45+Lin−NK1.1−CD90.2+GATA3− RORγt+) are shown (left). Numbers in flow cytometry plots represent percentages of Ki-67+ cells in each gate. Percentage and number of Ki-67+ cells among colonic ILC3s were analyzed by flow cytometry (right). Each symbol (FIGS. 1C-1D) represents an individual mouse. Data are representative of 3 independent experiments (a,b,d) and 4 independent experiments (FIG. 1C). Data represent means±s.e.m. * P<0.05, ** P<0.01, *** P<0.001, **** P<0.0001 (one-way ANOVA with Tukey's (FIG. 1A) or Dunnett's (FIG. 1B) multiple comparisons test, two-tailed Mann-Whitney test (FIGS. 1C-1D)).

FIG. 2A-2E shows Ffar2 regulates colonic ILC3 proliferation and ILC3-derived IL-22 production in a cell-intrinsic manner. FIG. 2A shows colonic RORγt+ ILC3s from RORγt−Cre Ffar2^{fl/fl} (n=8) mice or their littermate control Ffar2^{fl/fl} mice (n=8) were analyzed by flow cytometry. Isolated colonic LP cells were stained and gated on live CD45+Lin−NK1.1−CD90.2+GATA3− RORγt+ for colonic ILC3s. FIG. 2B shows Ki-67 expression in colonic ILC3s from RORγt−Cre Ffar2^{fl/fl} (n=6) mice or their littermate control Ffar2^{fl/fl} mice (n=5). Flow cytometry plots of live ILC3s (gated on live CD45+Lin−NK1.1−CD90.2+RORγt+) are shown (left). Numbers in flow cytometry plots represent percentages of Ki-67+ cells. Percentage and number of Ki-67+ cells among colonic ILC3s are shown (right). FIG. 2C shows intracellular cytokine staining of colonic RORγt+ ILC3s from RORγt−Cre Ffar2^{fl/fl} mice (n=8) or littermate control Ffar2^{fl/fl} mice (n=8). Representative flow cytometry plot for IL-22 and IL-17A staining in colonic ILC3s (gated on live CD45+Lin−NK1.1−CD90.2+RORγt+) (left). Percentage and number of IL-22 or IL-17A-producing ILC3s among colonic ILC3s are shown (right). FIG. 2D shows flow cytometry analysis of RORγt expression in colonic RORγt+ ILC3s from RORγt−Cre Ffar2^{fl/fl} (n=9) mice or their littermate control Ffar2^{fl/fl} mice (n=8). Histogram represents mean fluorescence intensity (MFI) of RORγt in colonic ILC3s from indicated mice. Gray shaded area indicates isotype-matched control antibody. Bar graph depicts the average MFI of RORγt in colonic ILC3s. FIG. 2E shows intracellular Ahr levels in colonic RORγt+ ILC3s or IL-22− producing ILC3s from RORγt−Cre Ffar2^{fl/fl} (n=9) mice or control Ffar2^{fl/fl} mice (n=8). MFI of Ahr in indicated cells was measured by flow cytometry. Each symbol (a,b,c) represents an individual mouse. Data are representative of 4 independent experiments (FIG. 2A), 3 independent experiments (FIG. 2B), 4 independent experiments (FIG. 2C) or are pooled from 2 independent experiments with a total of at least 4 mice per group (FIG. 2D-2E). Data represent means±s.e.m. * P<0.05, ** P<0.01, *** P<0.001 (unpaired two-tailed Student's t-test (FIG. 2A-2E), two-tailed Mann-Whitney test (FIG. 2B) as these data were not normally distributed, this non-parametric test was employed).

FIG. 3A-3G shows Ffar2 influences CCR6+ILC3 expansion and function. FIG. 3A shows flow cytometry analysis of colonic ILC3 subsets in RORγt−Cre Ffar2^{fl/fl} (n=5) mice or their littermate control Ffar2^{fl/fl} mice (n=5). Representative flow cytometry plot of CCR6+ ILC3s (gated on live CD45+Lin−NK1.1−CD90.2+RORγt+CCR6+NKp46−), CCR6− ILC3s (gated on live CD45+Lin−NK1.1−CD90.2+RORγt+CCR6−NKp46−), and NKp46+ ILC3s (gated on live CD45+Lin−NK1.1−CD90.2+RORγt+CCR6−NKp46+) are shown (left). Numbers of colonic ILC3 subsets are shown (right). FIG. 3B shows Ki-67 expression in colonic ILC3 subsets from RORγt−Cre Ffar2^{fl/fl} (n=5) mice or littermate control Ffar2^{fl/fl} mice (n=5). Flow cytometry plots of Ki-67+ cells in CCR6+ ILC3s and CCR6− ILC3s are shown (left). Numbers in flow cytometry plots represent percentages of Ki-67+ cells. Numbers of colonic Ki-67+ cells in colonic ILC3 subsets are shown (right). FIG. 3C shows IL-22− producing ILC3 subsets in RORγt−Cre Ffar2^{fl/fl} (n=5) mice or Ffar2^{fl/fl} mice (n=5). (FIGS. D-G) Distribution of Ffar2-expressing ILC3s in colonic lymphoid tissues of RORγt−Cre Ffar2^{fl/fl} mice (n=4) or littermate control Ffar2^{fl/fl} mice (n=4). RNA in situ hybridization of Ffar2 (magenta) and Rorc (blue) and immunofluorescence staining of CD3 (green) were performed on colon tissue section. FIG. 3D shows representative images of colonic ILC3s (RORγt+CD3−) in a colonic patch and a colonic solitary intestinal lymphoid tissue (SILT) from RORγt−Cre Ffar2^{fl/fl} mice or Ffar2^{fl/fl} mice. Scale bars, 100 μm (colonic patch); 20 μm (colonic SILT). FIG. 3E shows representative images of Ffar2 expression on colonic ILC3s in colonic lymphoid tissues. Scale bars, 10 μm. FIG. 3F shows number of colonic patches and colonic SILTs in the colon from RORγt−Cre Ffar2^{fl/fl} mice or littermate control Ffar2^{fl/fl} mice. FIG. 3G shows quantification of colonic ILC3s in colonic lymphoid tissues of RORγt-Cre Ffar2^{fl/fl} mice or Ffar2^{fl/fl} mice. Number of ILC3s (RORγt+CD3−) in a colonic patch or SILT was counted. Data are representative of 3 independent experiments (FIGS. A-C) or combined from 2 independent experiments (FIGS. 3D-3G). Data represent means±s.e.m. * P<0.05, ** P<0.01 (two-tailed Mann-Whitney test).

FIG. 4A-4F shows Ffar2 regulates colonic ILC3-derived IL-22 via AKT and STAT3 activation. FIG. 4A-4E shows flow cytometry analysis of phosphorylation of AKT, p38, ERK and STAT3 in sorted colonic ILC3s. FIG. 4A shows phosphorylation of AKT, p38 and ERK in sorted colonic ILC3s (gated on live CD45+Lin−NK1.1− NKp46+/−CD90.2+KLRG1−) from WT (n=20 for each signal protein) or Ffar2−/− mice (n=20 for each signal protein). Percentage of phosphorylated cells and MFI level are shown. FIG. 4B shows phosphorylation of AKT in sorted colonic ILC3s from WT mice that were cultured with Ffar2 agonist (n=24). FIG. 4C shows STAT3 activation in sorted colonic ILC3s from WT (n=24) or Ffar2−/− mice (n=24). FIG. 4D shows phosphorylation of STAT3 in sorted colonic ILC3s from WT mice that were cultured with Ffar2 agonist (n=24). FIG. 4E shows pSTAT3 expression in sorted colonic ILC3s cultured with Ffar2 agonist and AKT inhibitor (VIII) (n=30) FIG. 4F shows IL-22 expression in sorted ILC3s cultured with Ffar2 agonist, AKT inhibitor (VIII) or STAT3 inhibitor (S3I) (AKT inhibitor, n=24; STAT3 inhibitor, n=24). IL-22 expression was normalized to the housekeeping gene Actb. Data are pooled from 4 independent experiments. Data represent means±s.e.m. * P<0.05, ** P<0.01 (two-tailed Student's t-test).

FIG. 5A-5F shows Ffar2-expressing ILC3s protect against DSS-induced colonic injury and inflammation. FIG. 5A shows gene expression in epithelial cells of RORγt−Cre Ffar2^{fl/fl} (n=7) mice compared to littermate control Ffar2^{fl/fl} mice (n=6). FIG. 5B-5F shows RORγt−Cre Ffar2^{fl/fl} (n=12) and Ffar2^{fl/fl} mice (n=12) were given 3% DSS solution for 5 days followed by 2 days of normal water. Mice were sacrificed on day 7. FIG. 5B shows body weight changes were measured daily. FIG. 5C shows colon length. FIG. 5D shows histologic colitis score. FIG. 5E-5F shows flow cytometry analysis of colonic ILC3s and IL-22− producing ILC3s. Colonic LPs were isolated from RORγt−Cre Ffar2^{fl/fl} and Ffar2^{fl/fl} mice on day 7 after DSS treatment, stained with antibodies against RORγt+ILC3s and IL-22− producing RORγt+ ILC3s. Percentage and number of colonic ILC3s (gated on live CD45+Lin−NK1.1−CD90.2+RORγt+) and IL-22+ILC3s were shown. Each symbol (FIG. 5E-5F) represents an individual mouse. Data are combined from 2 independent experiments (FIG. 5A) or 3 independent experiments (FIG. 5B-D) or are representative of 3 independent experiments (FIG. 5E-5F). Data represent means±s.e.m. * P<0.05, ** P<0.01, *** P<0.001(unpaired two-tailed Student's t-test (FIGS. 5A-5F), two-tailed Mann-Whitney test (FIGS. 5E-5F)).

FIGS. 6A-6E shows Ffar2-expressing ILC3s are required for host defense against C. rodentium infection. RORγt−Cre Ffar2^{fl/fl} (n=15) and their littermate control Ffar2^{fl/fl} mice (n=14) were infected with 4×109 CFU of C. rodentium by oral gavage. Mice were sacrificed on day 7. FIG. 6A shows body weight changes were measured daily. FIG. 6B shows colon length. FIG. 6C shows bacterial load in spleen (left) and liver (right) on day 7. FIGS. 6D-6E shows flow cytometry analysis of colonic ILC3s and IL-22− producing ILC3s. Colonic LPs were isolated from RORγt−Cre Ffar2^{fl/fl} and Ffar2^{fl/fl} mice on day 7 after C. rodentium infection, stained with antibodies against RORγt+ ILC3s and IL-22+RORγt+ ILC3s. Percentage and number of colonic ILC3s (gated on live CD45+Lin−NK1.1−CD90.2+RORγt+) and IL-22+ILC3s were shown. Each symbol (FIGS. 6D-E) represents an individual mouse. Data are combined from 3 independent experiments (FIGS. 6A-C) or are representative of 3 independent experiments (FIGS. 6D-6E). Data represent means±s.e.m. * P<0.05, ** P<0.01 (unpaired two-tailed Student's t-test (FIGS. 6A-6B), two-tailed Mann-Whitney test (FIG. 6C-6E)).

FIG. 7A-7F shows colonic ILC3s may be a target of Ffar2 agonism. FIG. 7A-7B shows a T cell-transfer colitis model. C57BL/6 Rag2−/− mice were injected with naïve CD4+ T cells (CD3+CD4+CD25−CD45RBhi, 5×105 cells/mouse) alone or in combination with Treg cells (CD3+CD4+Foxp3+, 1×105 cells/mouse) from WT mice. After injection, mice received an Ffar2 agonist or sodium propionate for 6 weeks. FIG. 7A shows histologic colitis score. Naïve CD4+ T cells only (n=6); Foxp3+ Tregs (n=7); Ffar2 agonist (n=7); sodium propionate (n=7) FIG. 7B shows the number of colonic Treg cells (gated on live CD45+CD3+CD4+Foxp3+) from T cell-transfer colitis mice. Naïve CD4+ T cells only (n=6); Foxp3+ Tregs (n=7); Ffar2 agonist (n=7); sodium propionate (n=7) FIG. 7C shows flow cytometry analysis of colonic RORγt+ ILC3s from WT mice fed with SCFAs. N, sodium chloride as a control (n=18 (n=6 for each SCFA)); A, sodium acetate (n=10); P, sodium propionate (n=8); B, sodium butyrate (n=8). Percentage (left) and number (right) of colonic ILC3s (gated on live CD45+Lin−NK1.1−CD90.2+RORγt+) are shown. FIG. 7D shows flow analysis of colonic RORγt+ ILC3s from WT (n=7) or germ-free (GF) WT mice (n=8). FIG. 7E shows number of colonic RORγt+ ILC3s from GF-WT fed with Ffar2 agonist for 1 week. Control (n=4) and Ffar2 agonist (n=6) FIG. 7F shows number of colonic RORγt+ ILC3s from GF-WT fed with sodium acetate (n=5) or sodium chloride (4) as a control for 2-week. Each symbol (FIG. 7A-F) represents an individual mouse. Data are pooled from 3 independent experiments (FIG. 7A-B, FIG. 7D), 6 independent experiments (FIG. 7C) or 2 independent experiments (FIG. 7E-F). Data represent means±s.e.m. * P<0.05, ** P<0.01, *** P<0.001, **** P<0.0001 (one-way ANOVA with Tukey's (FIG. 7A-B) or Dunnett's (FIG. 7C) multiple comparisons test, two-tailed Mann-Whitney test (FIG. 7D-F)).

FIG. 8A-8F shows FFar2 does not affect total colonic ILCs, GATA3+ILC2s or RORγt+CD4+ T cells. FIG. 8A shows flow analysis of colonic ILCs (gated on live CD45+Lin−NK1.1−CD90.2+) from RORγt−Cre Ffar2^{fl/fl} (n=8) or littermate control Ffar2^{fl/fl} mice (n=8). FIG. 8B shows representative flow cytometry plot of Ki-67 in colonic GATA3+ILC2s (gated on live CD45+Lin−NK1.1−CD90.2+GATA3+RORγt−) from RORγt−Cre Ffar2^{fl/fl} (n=6) mice or their littermate control Ffar2^{fl/fl} mice (n=5). Numbers in flow cytometry plots represent percentages of Ki-67+ cells. FIG. 8C shows flow cytometry analysis of colonic RORγt+CD4+ T cells in RORγt−Cre Ffar2^{fl/fl} mice (n=10) or littermate control Ffar2^{fl/fl} mice (n=11). Frequency of RORγt+ CD4+ T cells (gated on live CD45+CD3+CD4+RORγt+) within CD4+ T cells (upper) and total number of RORγt+CD4+ T cells (bottom) are shown. FIG. 8D shows intracellular cytokine staining of IL-22 and IL-17A in colonic RORγt+CD4+ T cells from RORγt−Cre Ffar2^{fl/fl} mice (n=10) or littermate control Ffar2^{fl/fl} mice (n=11). FIG. 8E shows Il23 expression in colonic lamina propria from RORγt−Cre Ffar2^{fl/fl} mice (n=4) or littermate control Ffar2^{fl/fl} mice (n=4). mRNA expression was normalized to the housekeeping gene Actb. FIG. 8F shows I123r expression in sorted colonic ILC3s (gated on live CD45+Lin−NK1.1−NKp46+/−CD90.2+KLRG1−) from WT (n=15) or Ffar2−/− mice (n=18). mRNA expression was normalized to Actb. Each symbol (FIG. 8B-8C) represents an individual mouse. Data are representative of 4 independent experiments (FIG. 8A) and 3 independent experiments (FIG. 8B) or are pooled from 3 independent experiments (FIGS. 8C-8D) and 6 independent experiments (FIGS. 8E-8F). Data represent means±s.e.m.

FIG. 9A-9H shows Ffar2 influences colonic RORγt+ CCR6+ ILC3 expansion. FIG. 9A shows RORγt and Ffar2 mRNA expression in colonic ILC3 subsets from WT mice (n=18 per each gene). mRNA expression was normalized to the housekeeping gene Actb. Colonic CCR6+ ILC3s (gated on live CD45+Lin−NK1.1−NKp46−CD90.2+KLRG1−CCR6+) and CCR6− ILC3s (gated on live CD45+Lin−NK1.1−NKp46+/−CD90.2+KLRG1−CCR6−) were isolated with a FACSAria. FIG. 9B shows flow cytometry analysis of RORγt expression in colonic ILC3 subsets from RORγt−Cre Ffar2^{fl/fl} (n=5) mice or their littermate control Ffar2^{fl/fl} mice (n=5). MFI, mean fluorescence intensity. FIG. 9C-G shows distribution of Ffar2-expressing ILC3s in colonic lymphoid tissues of WT mice (n=3) or their littermate Ffar2−/− mice (n=3). RNA in situ hybridization of Ffar2 (magenta) and Rorc (blue) and immunofluorescence staining of CD3 (green) were performed on colon tissue section. FIG. 9C shows representative images of colonic ILC3s (RORγt+CD3−) in a colonic patch and a colonic solitary intestinal lymphoid tissue (SILT) from WT mice or Ffar2−/− mice. Scale bars, 100 μm (colonic patch); 20 μm (colonic SILT). FIG. 9D-9E shows representative images of Ffar2 expression on colonic ILC3s in a colonic patch from WT mice or Ffar2−/− mice. Scale bars, 10 μm. Arrows indicate FFar2-sufficient ILC3 (FIG. 9D) and Ffar2-deficient ILC3 (FIG. 9E). FIG. 9F shows number of colonic patches and colonic SILTs in the colon from WT mice or Ffar2−/− mice. FIG. 9G shows quantification of colonic ILC3s in colonic lymphoid tissues of WT mice or Ffar2−/− mice. Number of ILC3s (RORγt+CD3−) in a colonic patch or colonic SILT was counted. FIG. 9H shows Tbx21, Notch and Tox mRNA expression in colonic CCR6+ ILC3s and CCR6− ILC3s from WT (n=18) or Ffar2−/− mice (n=18). Samples were normalized using Hprt1, Gapdh and Eefla1 as housekeeping genes. Data represent means±s.e.m. Data are pooled from 6 independent experiments (FIG. 9A, FIG. 9H) or are representative of 3 independent experiments (FIG. 9B) and 2 independent experiments (FIG. 9C-9G). * P<0.05, ** P<0.01 (two-tailed Mann-Whitney test).

FIG. 10A-10F shows FFar2 signaling activates AKT and STAT3 in sorted colonic ILC3s. FIG. 10A shows representative flow cytometry histograms of phosphorylated AKT, p38 and ERK in sorted colonic ILC3s (gated on live CD45+Lin−NK1.1−NKp46+/−CD90.2+KLRG1−) from WT (n=20 for each signal protein) or Ffar2−/− mice (n=20 for each signal protein). Bold lines, WT ILC3; dashed lines, Ffar2−/− ILC3s; gray shaded area, isotype-matched control antibody. FIG. 10B shows representative flow cytometry histogram of phosphorylated AKT in sorted colonic ILC3s from WT mice that were cultured with Ffar2 agonist (n=24). Bold line, Ffar2 agonist; dashed line, vehicle (DI water). FIG. 10C shows phosphorylated p38 and ERK in sorted colonic ILC3s from WT mice that were cultured with Ffar2 agonist (n=24 for each signal protein). Percentage of phosphorylated p38+ and ERK+ cells and MFI levels are shown. FIG. 10D shows IL-22 expression in colonic ILC3s from WT mice (n=15) or Ffar2−/− mice (n=15). mRNA expression was normalized to the housekeeping gene Actb. FIG. 10E shows representative flow cytometry histogram of phosphorylation of STAT3 in sorted colonic ILC3s from WT (n=24) or Ffar2−/− mice (n=24). FIG. 10F shows representative flow cytometry histogram of pSTAT3 in sorted colonic ILC3s from WT mice (n=24) that were cultured with Ffar2 agonist (n=24). Data represent means±s.e.m. Data reflect 4 independent experiments (FIG. 10A-C, FIGS. 10E-10F) and 6 independent experiments (FIG. 10D). Data represent means±s.e.m. ** P<0.01 (two-tailed Student's t-test).

FIGS. 11A-11H demonstrates that Ffar2 regulates colonic ILC3-derived IL-22 via AKT and STAT3 activation. FIG. 11A shows that Il22 mRNA expression in sorted ILC3s cultured with acetate (A) (10 mM), propionate (P) (10 mM), or Ffar2 agonist (10 mM) (A, N=21; P, N=21; Ffar2 agonist, N=24). FIG. 11B shows Il22 mRNA expression in sorted ILC3s cultured with Ffar2 agonist (10 mM), Gi/o inhibitor (pertussis toxin [PTX]) (500 ng/mL), or a Gq inhibitor (YM-254890) (1 mM) overnight (Gi/o inhibitor, N=24; Gq inhibitor, N=24). FIG. 11C shows Il22 mRNA expression in sorted ILC3s cultured with P (10 mM), PTX (500 ng/mL) or YM-254890 (1 mM) overnight (PTX, N=24; YM-254890, N=24). FIG. 11D-11F shows flow analysis of AKT, p38, ERK, and STAT3 phosphorylation in sorted colonic ILC3s. FIG. 11D shows AKT, p38, and ERK phosphorylation in sorted colonic ILC3s from WT (N=20 for each protein) or Ffar2−/− mice (N=20 for each protein). FIG. 11E shows STAT3 activation in sorted colonic ILC3s from WT (N=24) or Ffar2−/− mice (N=24). FIG. 11F shows AKT, p38, ERK, and STAT3 phosphorylation in sorted colonic ILC3s from mice cultured with P (10 mM) (N=21 for each protein) or Ffar2 agonist (10 mM) (N=24 for each protein). FIG. 11G shows Il22 mRNA expression in sorted ILC3s cultured with Ffar2 agonist (10 mM), AKT (10 mM), or STAT3 inhibitor (10 mM) (AKT inhibitor, N=24; STAT3 inhibitor, N=24). FIG. 11H shows STAT3 activation in sorted colonic ILC3s cultured with AKT inhibitor (10 mM) before stimulation with Ffar2 agonist (10 mM) (N=30). Data pooled from 4 independent experiments. Data reflect 3 independent flow cytometry sorting sessions with cells harvested from 5-7 mice per Ffar2 ligand (FIG. 11A), 4 independent FACS sessions with cells harvested from 6 mice per inhibitor (FIGS. 11B, C, G) or 5-6 mice per protein (FIGS. 11D, E, F, H). Data (bars) represent mean±SEM *p<0.05, **p<0.01, ***p<0.001, two-tailed Student's t test. See also FIGS. 12A-12H.

FIG. 12A-12H demonstrates that Ffar2 agonism distinctly affects ILC3 proliferation and activates AKT, ERK and STAT3 in sorted colonic ILC3s. FIG. 12A shows the percentage of in vitro BrdU+ incorporation in sorted colonic ILC3s that were cultured with acetate (A) (10 mM) or propionate (P) (10 mM) and BrdU (10 μM) overnight (acetate, n=20; propionate, n=20). FIG. 12B-12C shows representative flow cytometry histograms and MFI levels of phosphorylated AKT, p38, ERK, STAT3 in sorted colonic ILC3s from WT mice (n=20 for each protein) or Ffar2−′− mice (n=20 for each protein). Bold lines, WT (Ffar2+′+) ILC3; dashed lines, Ffar2−′− ILC3s; gray shaded area, isotype-matched control antibody. FIG. 12D shows phosphorylated AKT and STAT3 in sorted colonic ILC3s from WT mice that were cultured with the Ffar2 agonist (n=24 for each protein). Bold line, Ffar2 agonist; dashed line, vehicle (DI water). Representative flow cytometry histograms of pAKT and pSTAT3 are shown (upper panel). MFI levels of pAKT and pSTAT3 are shown (lower panel). FIG. 12E shows phosphorylation of AKT, p38, ERK and STAT3 in sorted colonic ILC3s from Ffar2−/− mice that were cultured the Ffar2 agonist (10 μM) for 30 min (n=24 for each protein). FIG. 12F shows phosphorylation of ERK in sorted colonic ILC3s from WT mice that were cultured with propionate (10 mM) for 1 h (n=20). FIG. 12G shows phosphorylation of STAT3 in sorted colonic ILC3s from WT mice that were cultured with propionate (10 mM) for 1 h (n=20 for each protein). FIG. 12H shows Il22 mRNA expression in sorted ILC3s cultured with the propionate (10 mM) or ERK inhibitor (10 μM) overnight (n=24). Data show means±s.e.m. Data reflect 4 independent FACS sessions with cells harvested from 5 mice (FIGS. 12A, 12B, 12C, 12F, 12G) or 6 mice (FIGS. 12D, 12E, 12H) per each protein and 4 independent flow cytometry analyses. *p<0.05, (two-tailed Student's t-test).

### DETAILED DESCRIPTION

Briefly, the methods and compositions described herein relate to methods and agents for treating or preventing a gastrointestinal disease. In one aspect, the method comprises: administering to a subject in need thereof an agent that increases the level or activity of Free Fatty Acid Receptor 2 (Ffar2) in the subject.

### Some Selected Definitions:

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with gastrointestinal disease, e.g. ulcerative colitis. The term “treating” includes reducing or alleviating at least one adverse effect or symptom of gastrointestinal disease, for example, diarrhea, bleeding, loss of appetite, discomfort, or vomiting. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein “preventing” or “prevention” refers to any methodology where the disease state does not occur due to the actions of the methodology (such as, for example, administration of an agent as described herein). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g. a subject who is not treated with the methods or compositions described herein).

As used herein, the terms “administering,” and “injecting” are used interchangeably in the context of the placement of an agent (e.g. a small molecule) described herein, into a subject, by a method or route which results in at least partial localization of the agent at a desired site, such as the gastrointestinal tract or a region thereof, such that a desired effect(s) is produced (e.g., increase Ffar2 level or activity). The agent described herein can be administered by any appropriate route which results in delivery to a desired location in the subject. The half-life of the agent after administration to a subject can be as short as a few minutes, hours, or days, e.g., twenty-four hours, to a few days, to as long as several years, i.e., long-term. In some embodiments of any of the aspects, the term “administering” refers to the administration of a pharmaceutical composition comprising one or more agents. The administering can be done by direct injection (e.g., directly administered to a target cell or tissue), subcutaneous injection, muscular injection, oral, or nasal delivery to the subject in need thereof. Administering can be local or systemic.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a gastrointestinal disease, or has never received treatment for a gastrointestinal disease. A subject can have previously been diagnosed with having a gastrointestinal disease, or has never been diagnosed with a gastrointestinal disease.

The term “agent” as used herein means any compound or substance such as, but not limited to, a small molecule, nucleic acid, polypeptide, peptide, drug, ion, short chain fatty acid (SCFA), etc. An “agent” can be any chemical, entity or moiety, including without limitation, synthetic and naturally-occurring proteinaceous and non-proteinaceous entities. In some embodiments of any of the aspects, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

The agent can be a molecule from one or more chemical classes, e.g., organic molecules, which may include organometallic molecules, inorganic molecules, genetic sequences, etc. Agents may also be fusion proteins from one or more proteins, chimeric proteins (for example domain switching or homologous recombination of functionally significant regions of related or different molecules), synthetic proteins or other protein variations including substitutions, deletions, insertion and other variants.

The term “derivative” as used herein means any chemical, conservative substitution, or structural modification of an agent. The derivative can improve characteristics of the agent or small molecule such as pharmacodynamics, pharmacokinetics, absorption, distribution, delivery, targeting to a specific receptor, or efficacy. For example, for a small molecule, the derivative can consist essentially of at least one chemical modification to about ten modifications. The derivative can also be the corresponding salt of the agent (e.g. sodium propionate as a derivative of propionate). The derivative can be the pro-drug of the small molecule as described herein.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level. For example, increasing activity can refer to activating Ffar2 receptors or increasing levels of Ffar2 directly or indirectly.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a gastrointestinal disease, or a biological sample that has not been contacted with an agent or composition disclosed herein).

As used herein, an “appropriate control” refers to an untreated, otherwise identical cell or population (e.g., a biological sample that was not contacted by an agent or composition described herein, or not contacted in the same manner, e.g., for a different duration, as compared to a non-control cell). In some embodiments of any of the aspects, an appropriate control would be the level of Ffar2 activity in an otherwise identical sample that is not contacted by an agent or composition described herein, or is the level of Ffar2 activity in a subject prior to administration of an agent or composition. Further, an appropriate control can be the level of Ffar2 activity in a healthy subject, e.g., an individual that does not have a gastrointestinal disease. One skilled in the art can determine the activity of Ffar2 using functional readouts of Ffar2's activity, for example, by measuring/assessing the secretion of IL-22. One skilled in the art can assess/measure the protein and mRNA levels of Ffar2 and downstream targets or secretions from the cells of interest, e.g., using western blotting or PCR-based assays, respectively.

The term “pharmaceutically acceptable” can refer to compounds and compositions which can be administered to a subject (e.g., a mammal or a human) without undue toxicity.

As used herein, the term “secreting” or “secretion” are used interchangeably to refer to the ability of a cell or tissue to release a protein, molecule, nucleic acid, or vesicle. For example, ILC3s can secrete interleukins (e.g. IL-22), small proteins of ˜5-20 kDa that modulate cell signaling such as immune responses to infections.

As used herein, “detecting” is understood to mean that an assay was performed for a specific target or protein (e.g. Ffar2). The amount of target detected can be none or below the level of detection of the assay. Examples of assays include but are not limited to, flow cytometry, immunohistochemistry, real time or reverse transcriptase-PCR, Western blotting, enzyme-linked immunosorbent assay (ELISA), or any other assay known in the art.

As used herein, the term “modulates” refers to an effect including increasing or decreasing a given parameter as those terms are defined herein.

As used herein, the term “contacting” when used in reference to a cell or organ, encompasses both introducing or administering an agent, surface, hormone, etc. to the cell, tissue, or organ in a manner that permits physical contact of the cell with the agent, surface, hormone etc., and introducing an element, such as a genetic construct or vector, that permits the expression of an agent, such as a miRNA, polypeptide, or other expression product in the cell. It should be understood that a cell genetically modified to express an agent, is “contacted” with the agent, as are the cell's progeny that express the agent.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

### Innate Lymphoid Cell Function in the Gut:

The innate lymphoid cell (ILC) family plays a significant role in immunity, inflammation, tissue homeostasis, and repair. Generally, ILCs are classified into three groups on the basis of signature transcription factors and distinct effector cytokines: Group 1 ILCs (TLC's) require T-bet and produce interferon-γ (IFN-γ), Group 2 ILCs (ILC2s) express GATA3 and produce the type 2 cytokines interleukin 5 (IL-5) and IL-13, and Group 3 ILCs (ILC3s) are a heterogeneous population expressing transcription factor RAR-related orphan receptor gamma t (RORγt) and have the ability to produce IL-22 and/or IL-17.

ILC3s are enriched in the intestine, where they maintain gut homeostasis by orchestrating: lymphoid organ development, containment of commensal bacterial, tissue repair, host defense and regulation of adaptive immunity. ILC3s process signals from other cells and soluble mediators within their local tissue microenvironment. Environmental cues, such as microbial, dietary, and neuronal signals, regulate ILC3s through cell-intrinsic receptors.

As used herein, the terms “group 3 innate lymphoid cells” or “innate lymphoid cells” or “ILC3s” are used interchangeably to refer to immune cells in the gut responsible for maintaining tissue function, immunity, and repair. Innate lymphoid cells (ILCs) are classified into three groups on the basis of signature transcription factors and distinct effector cytokines: Group 1 ILCs (ILC1s) consist essentially of T-bet and produce interferon-γ (IFN-γ), Group 2 ILCs (ILC2s) consist essentially of GATA3 and produce the type 2 cytokines interleukin 5 (IL-5) and IL-13, and Group 3 ILCs (ILC3s) consist essentially of a heterogeneous population expressing transcription factor RAR-related orphan receptor gamma t (RORγt) and have the ability to produce IL-22 and/or IL-17. ILC3s are typically enriched in the intestine, where they maintain gut homeostasis by orchestrating: lymphoid organ development, containment of commensal bacterial, tissue repair, host defense and regulation of adaptive immunity. ILC3s can on occasion be divided into two subsets based on CC chemokine receptor type 6 (CCR6) expression. Both CCR6+ ILC3s and CCR6− ILC3s produce IL-22, a key cytokine that is essential for recovery from tissue damage and protection against intracellular bacteria. ILC3 proliferation as described herein can be necessary for protection from infection.

In one aspect, described herein is a method of increasing the levels of ILC3s in the gastrointestinal tract, the method comprises: administering an agent that increases the level or activity of Ffar2.

In one embodiment of any of the aspects, the agent protects from an infection or inflammation.

In another embodiment of any of the aspects, the agent described herein induces an increase in the number of group 3 innate lymphoid cells (ILC3s). In another embodiment, of any of the aspects, the agent induces an increase in T-regulatory cells (Tregs). In another embodiment of any of the aspects, the ILC3s are CC chemokine receptor type 6 (CCR6) positive or CCR6 negative cells. The ILC3s as described herein can be present in any tissue in the body or systemically. Exemplary tissues where ILC3s are present include but are not limited the esophagus, stomach, small intestine, large intestine, or colon.

ILC3s can be characterized by any method known in the art such as flow cytometry, immunohistochemistry, real time PCR using cell surface markers, transcription factors, or secretions specific to ILC3s. Markers of ILC3s include but are not limited to Ffar2, cluster of differentiation (CD) CD45, CD90.2, CD11b, CD11c, protein gamma response 1 (Gr-1), major histocompatibility complex (MHC) MHC Class II, Killer cell lectin-like receptor subfamily G member 1 (KLRG1), CD3c, CD4, interleukins (IL) IL-22, IL-17, CCR6, Aryl hydrocarbon receptor (Ahr), RAR-related orphan receptor gamma (RORγ), RORγt, IL-1β, interferon γ (IFN γ) or any other ILC3 marker known in the art.

In the context of the methods described herein, the term “functional property,” as applied to an innate lymphoid cell(s), ILC3s, or culture of ILC3s, refers to any of the parameters described herein as measures of innate lymphoid cell function or ILC3 function. A “change in functional property” as described herein is indicated by a statistically significant increase or decrease in a functional property with respect to a reference level or appropriate control.

In addition to ILC3s, the gastrointestinal tract can comprise other immune cells, such as regulatory T cells or “T_{regs}.” T_{regs} are a subpopulation of T cells that modulate the immune system, and prevent infections (e.g. gastrointestinal infections). The T_{regs} express biomarkers CD4, Fox3P, and CD25. T_{regs} can be found in the colon. T_{regs} produce and secrete a number of cytokines including IL-35 and IL-10 that regulate immune cell function. As described in the working examples, Ffar2 can also regulate T_{reg} cell function and expansion in the colon.

### Ffar2 and Gut Homeostasis:

In one aspect, described herein is a method of increasing the level or activity of FFar2 in a subject.

The Free Fatty Acid Receptor 2 (Ffar2, Ffar2 receptor, GPR43) is a G-protein coupled receptor that is expressed in the gut, adipose tissue, pancreas, spleen, lymph nodes, bone marrow, and peripheral blood mononuclear cells, among others. Specifically, Ffar2 can couple with G_{i/o} or G_{q} proteins and Ffar2 activation can inhibit cAMP and/or stimulate Ca²⁺ influx, eliciting intracellular signal cascades that regulate numerous cell-specific functions. For example, Ffar2 is involved in cell signaling events related to inflammation in response to infections, injury, and the like. Sequences for FFAR2, also known as GPR43, are known for a number of species, e.g., human FFAR2 (NCBI Gene ID: 2867 and NCBI Reference Sequence NC_000019.10) polypeptide and mRNA (e.g., NCBI Reference Sequence: NM_005306.2). Ffar2 can refer to human Ffar2, including naturally occurring variants, molecules, genetically engineered Ffar2, and alleles thereof. Ffar2 refers to the mammalian Ffar2 of, e.g., mouse, rat, rabbit, dog, cat, cow, horse, pig, and the like. The amino acid sequence of Ffar2 is shown in SEQ ID NO: 1. The gene sequence is shown in SEQ ID NO: 2.

Of the hundreds of bacterial metabolites in the gut, short-chain fatty acids (SCFAs) have emerged as substantial regulators of immune responses in the gut and systemically. SCFAs, which are produced in the colon through bacterial fermentation of dietary fiber can engage ‘metabolite-sensing’ G-protein-coupled receptors (GPCRs).

Ffar2 is a SCFA-sensing GPCR, and the functions of Ffar2 are broadly immunomodulatory. FFar2 plays a useful role in gut homeostasis and regulation of inflammation. Ffar2 also mediates colonic T_{reg} cell expansion and protects against T-cell transfer colitis.

Provided herein are methods related to Ffar2 signaling that affect colonic ILC3 proliferation and function in a cell-intrinsic manner. The methods described herein can modulate ILC3 regulation of gut inflammatory tone and pathogen defense.

As used herein, the terms “Ffar2 activity” or “activity of Ffar2” refers to the cellular functions of the Ffar2 receptor, for example, activation of Ffar2 in Group 3 innate lymphoid cells (ILC3s) results in the secretion of interleukins 22 (IL-22) and 17 (IL-17). As described herein, an increase in Ffar2 levels and activity results in the expansion of ILC3 populations in the gut. Ffar2 activity can further refer to the sensing of microorganism metabolites, maintenance of gut homeostasis, and resistance to infection. The activation of Ffar2 or an increase in Ffar activity as described herein can also refer to the secretion of other immunomodulatory molecules and proteins such as cytokines, interferons, and complement. Exemplary immunomodulatory molecules and proteins include but are not limited to IL-22, IL-17, IFNγ, granulocyte-macrophage colony-stimulating factor (GM-CSF), lymphotoxin (LT) alpha, and LT beta.

In one embodiment of any of the aspects, the agent described herein induces secretion of interleukin-22 (IL-22) and/or interleukin-17 (IL-17) from ILC3s. In another embodiment, of any of the aspects, the agent described herein induces secretion of IFNγ or IL-1β.

In another embodiment of any of the aspects, activating Ffar2 is increasing Ffar2 activity. The Ffar2 activity can be any function of the FFAR2 gene, gene product, or polypeptide, e.g., increased secretion of IL-22 by ILC3s. In another embodiment of any of the aspects, the activity of Ffar2 is increased by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control.

In another embodiment, of any of the aspects, activating Ffar2 is increasing Ffar2 levels in the cell, e.g., gene expression levels or gene product levels. In one embodiment of any of the aspects, Ffar2 levels are increased by at least 5%, 10%, 15%, 20%, 25%, 30%, 35%, 40%, 45%, 50%, 55%, 60%, 65%, 70%, 75%, 80%, 85%, 90%, 95%, 99%, or more as compared to an appropriate control.

### Gastrointestinal Diseases:

In one aspect of any of the embodiments, the methods, assays, and compositions described herein can be used to treat or prevent a gastrointestinal disease in a subject.

In another aspect of any of the embodiments, the methods described herein are methods and compositions for treating a subject having or diagnosed as having a gastrointestinal disease. The methods described herein comprise administering an agent that increases the levels or activity of Ffar2 as described herein.

In another aspect, described herein is a method of reducing inflammation in the gastrointestinal tract of a subject, the method comprises: administering to a subject an agent that increases the level or activity of Free Fatty Acid Receptor 2 (Ffar2) in the subject.

Subjects having a gastrointestinal disease can be identified by a physician using current methods of diagnosing a condition. Symptoms and/or complications of a gastrointestinal disease, which characterize this disease and aid in diagnosis are well known in the art and include but are not limited to, fatigue, pain, vomiting, nausea, upset stomach, diarrhea, etc. Tests that may aid in a diagnosis of, e.g. a gastrointestinal disease, include but are not limited example blood tests, non-invasive imaging, and/or tissue biopsy. A family history of a gastrointestinal disease will also aid in determining if a subject is likely to have the condition or in making a diagnosis of a gastrointestinal disease. Furthermore, a blood test or tissue biopsy will aid in diagnosing a gastrointestinal infection and which microorganism is responsible for said infection.

The methods and compositions described herein can further be used to treat or prevent other diseases in organs where Ffar2 is present. For example, such diseases would include but are not limited to, diabetes, obesity, pancreatitis, splenic rupture, biliary diseases, and the like.

As used herein the term “gastrointestinal disease” or “GI disease” refers to any disease that affects the gastrointestinal tract or gut. The gastrointestinal disease can cause at least one symptom of the disease. These symptoms can include but are not limited to, diarrhea, vomiting, nausea, upset stomach, pain, malaise, fever, weight loss, weight gain, bleeding, any change in the consistency or frequency of a bowel movement or stool, or any other symptom associated with a gastrointestinal disease in a subject. Non-limiting examples of gastrointestinal diseases include a gastrointestinal infection, inflammatory bowel disease (IBD), gastrointestinal injury, appendicitis, Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer.

In one embodiment of any of the aspects, the administering of the agent as described herein reduces inflammation of the gastrointestinal tract.

As used herein, the term “inflammation” or “inflamed” refers to activation or recruitment of the immune system or immune cells (e.g. T cells, B cells, macrophages, etc.). A tissue that has inflammation can become reddened, white, swollen, hot, painful, exhibit a loss of function, or have a film or mucus. Methods of identifying inflammation are well known in the art. Inflammation typically occurs following injury or infection by a microorganism.

In some embodiments of any of the aspects, the gastrointestinal disease described herein is selected from the group consisting of: a gastrointestinal infection, inflammatory bowel disease (IBD), gastrointestinal injury, appendicitis, Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer.

In some embodiments of any of the aspects, the gastrointestinal infection described herein is due to a microorganism. In another embodiment of any of the aspects, the microorganism is a bacterium, virus, fungus, parasite, yeast, prion, or any other microorganism known in the art.

As used herein, the term “microbe” or “microorganism” refers to an organism which is microscopic. A microbe can be a single-celled organism. In some embodiments of any of the aspects, a microbe is a bacterium. As used herein, the term “pathogen” refers to an organism or molecule that causes a disease or disorder in a subject. For example, pathogens include but are not limited to viruses, fungi, bacteria, parasites and other infectious organisms, or molecules therefrom, as well as taxonomically related macroscopic organisms within the categories algae, fungi, yeast and protozoa or the like.

In another embodiment of any of the aspects, the bacterium is a Clostridium, Staphylococcus, Streptococcus, Escherichia (e.g. E. coli), Mycobacterium, Pseudomonas, Burkholderiz, Trichomonas, Campylobacter, Shigella, Salmonella, Citrobacter, their species, or any other bacteria known in the art. In another embodiment of any of the aspects, the virus is influenza virus, coronavirus, retrovirus, or any other virus known in the art.

In one aspect of any embodiment, described herein is a method for treating or preventing a gastrointestinal disease, the method comprises: administering to a subject in need thereof an agent that increases the level or activity of Free Fatty Acid Receptor 2 (Ffar2) in the subject.

In another embodiment of any of the aspects, the subject has been previously diagnosed with having a gastrointestinal disease. In another embodiment of any of the aspects, the subject is diagnosed with a gastrointestinal disease prior to the administering of the agent. In another embodiment of any of the aspects, the subject is a mammal. In another embodiment of any of the aspects, the subject is a human.

### Agents

In one aspect, described herein is method of treating or preventing a gastrointestinal disease, the method comprises: administering to a subject an agent that increases the level or activity of Ffar2.

In one embodiment of any of the aspects, the agent preferentially binds to a Ffar2 receptor. In some embodiments of any of the aspects, the agent is an agonist of Ffar2, a partial agonist of Ffar2, or an allosteric modulator of Ffar2.

Efficacy of the agent or compositions described herein can be quantitated in several ways based on functional data as described herein (e.g. IL-22 secretion). The agent affinity for Ffar2 can be characterized by a dissociation constant (Kd). The Kd of the agent for the Ffar2 receptor can be about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M.

In another aspect of any embodiment, an agent that increases Ffar2 levels or activity is administered to a subject having or at risk of having a gastrointestinal disease.

In one embodiment of any of the aspects, the agent is selected from the group consisting of: a small molecule, an antibody, a peptide, a genome editing system, a vector, a nucleic acid, a miRNA, and a siRNA.

An agent described herein is considered effective for increasing the levels or activity of Ffar2 if, for example, upon administration, it increases the presence, amount, activity and/or level of Ffar2 in a cell.

An agent can increase or activate e.g., the transcription, or the translation of Ffar2 in the cell. An agent can increase the activity or alter the activity (e.g., such that the activity increases, is enhanced or occurs properly (e.g., as compared to wild-type Ffar2 activity), or occurs at an increased rate) of Ffar2 in the cell (e.g., Ffar2's expression).

The agent can function directly in the form in which it is administered. Alternatively, the agent can be modified or utilized intracellularly to produce something which increases Ffar2, such as introduction of a nucleic acid sequence into the cell and its transcription resulting in the production of the nucleic acid and/or protein agonist, partial agonist, modulator, or activator of Ffar2 within the cell.

In some embodiments of any of the aspects, the agent is any chemical, entity or moiety, including without limitation, synthetic and naturally-occurring non-proteinaceous entities. In certain embodiments, of any of the aspects, the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be identified from a library of diverse compounds.

As used herein, the term “small molecule” refers to a chemical agent which can include, but is not limited to, a short chain fatty acid, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In some embodiments of any of the aspects, the small molecule is a short chain fatty acid (SCFA). As used herein, the term “short chain fatty acid” or “SCFA” refers to a fatty acid or carboxylic acid consisting essentially of two to about ten carbon atoms in the carbon backbone and salts, esters, and pro-drugs thereof. Non-limiting examples of short chain fatty acids include propionic acid, acetic acid, butyric acid, formic acid, isobutyric acid, valeric acid, isovaleric acid, formate, acetate, propionate, butyrate, pentanoate, isobutyrate, valerate, isovalerate and pharmaceutically acceptable salts thereof (e.g. sodium propionate). SCFA or SCFAs can also refer to a mixture of propionic acid, acetic acid, and/or butyric acid.

In some embodiments of any of the aspects, the small molecule is a derivative of a SCFA. In some embodiments of any of the aspects, a pharmaceutical composition comprises a SCFA or mixture of SCFAs. In some embodiments of any of the aspects, SCFAs comprise a mixture of propionic acid, acetic acid, and/or butyric acid. In some embodiments of any of the aspects, the SCFAs can be engineered, synthesized, isolated, or processed. In some embodiments of any of the aspects, the SCFA is a pro-drug. In some embodiments of any of the aspects, SCFAs are isolated or produced by a living cell.

In some embodiments of any of the aspects, the small molecule is ES43012-SOD, trans-2-methylcrotonic acid, propiolic acid, angelic acid, compound 34, sodium acetate, sodium propionate, sodium butyrate, formate, pentanoate, (S)-2-(4-chlorophenyl)-3,3-dimethyl-N-(5-phenylthiazol-2-yl)butanamide, BTI-A-404, BTI-A-292, AZ1729, or any derivative thereof.

In some embodiments of any of the aspects, the small molecules described herein can preferentially bind or activate Ffar2 to increase Ffar2 levels and/or activity. For example, ES43012-SOD is a FFar2 agonist that, as provided herein, selectively promotes an increase in colonic ILC3 population frequency and number.

In some embodiments, the agent is ES43012-SOD or any derivative, or analog, thereof. In some embodiments, the agent described herein is Compound 1 in Patent Application Number: WO2011/076732 A1; which is incorporated herein by reference in its entirety.

Non-limiting examples of small molecules, peptides, and recombinant proteins that modulate Ffar2 activity are shown in TABLE 1 below. The small molecules, peptides, and recombinant proteins can be used in any combination and combined with any other agent or pharmaceutical composition described herein.

TABLE 1
Chemicals, Peptides, and Recombinant Proteins

| REAGENT or RESOURCE | SOURCE | IDENTIFIER |
|---|---|---|
| Ffar agonist | EPICS SA, Belgium | Compound 1- |
| | | WO2011/076732 A1 |
| Sodium Acetate | Sigma-Aldrich | S8750 |
| Sodium Propionate | Sigma-Aldrich | P5436 |
| Sodium Butyrate | Sigma-Aldrich | 303410 |
| Dithiothreitol | Sigma-Aldrich | 00-5523-00 |
| Penicillin/streptomycin | Corning | 30-002-CI |
| Collagenase D | Roche | 11088882001 |
| Collagenase A | Roche | 10103586001 |
| DNase I | Roche | 10104159001 |
| Dispase | StemCell | 07913 |
| | Technologies | |
| Phorbol mysristate acetate | Sigma-Aldrich | P8139-1MG |
| Ionomycin | Sigma-Aldrich | I0634-1MG |
| Brefeldin A Solution | BioLegend | 420601 |
| QIAzol | QIAGEN | 79306 |
| RNAlater | Sigma-Aldrich | R0901-100ML |
| Mm-Ffar2 probe | ACDBio | 433711 |
| TSA cyanine 3 | PerkinElmer | NEL744E001KT |
| TSA cyanine 5 | PerkinElmer | NEL745E001KT |
| Prolong Gold antifade | Life Technologies | P36934 |
| mounting | | |
| FITC dextran | Sigma-Aldrich | 46944-500MG-F |
| DSS | Thermo Scientific | J1448922 |
| Pertussis Toxin | Calbiochem | CAS 70323-44-3 |
| YM-254890 | Focus Biomolecules | 10-1590-0100 |
| AKT1/2 kinase inhibitor | Sigma-Aldrich | A6730-5MG |
| (VIII) | | |
| ERK kinase inhibitor | Sigma-Aldrich | P215-1MG |
| (PD98059) | | |
| STAT3 inhibitor | Sigma-Aldrich | SML0330-5MG |
| (S3I-201) | | |

Methods for screening small molecules are known in the art and can be used to identify a small molecule that is efficient at, for example, modulating Ffar2 activity or levels, given the desired target (e.g., Ffar2 receptor).

In various embodiments of any of the aspects, the agent is an antibody or antigen-binding fragment thereof, or an antibody reagent that is specific for Ffar2 or a regulator of Ffar2. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and Fab fragments, F(ab′)2, Fd fragments, Fv fragments, scFv, CDRs, and domain antibody (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, or IgM (as well as subtypes and combinations thereof). Antibodies can be from any source, including mouse, rabbit, pig, rat, and primate (human and non-human primate) and primatized antibodies. Antibodies also include midibodies, nobodies, humanized antibodies, chimeric antibodies, and the like.

In one embodiment of any of the aspects, the agent is a humanized, monoclonal antibody or antigen-binding fragment thereof, or an antibody reagent. As used herein, “humanized” refers to antibodies from non-human species (e.g., mouse, rat, sheep, etc.) whose protein sequence has been modified such that it increases the similarities to antibody variants produce naturally in humans. In one embodiment of any of the aspects, the humanized antibody is a humanized monoclonal antibody. In one embodiment of any of the aspects, the humanized antibody is a humanized polyclonal antibody. In one embodiment of any of the aspects, the humanized antibody is for therapeutic use.

In one embodiment of any of the aspects, the anti-Ffar2 antibody is any known anti-Ffar2 antibodies in the art, or any anti-Ffar2 antibodies that are yet to be discovered. Exemplary anti-Ffar2 antibodies known in the art include, but are not limited to, anti-Ffar2 antibodies sold by Thermo Fisher Scientific (Waltham, Mass.). In one embodiment of any of the aspects, the anti-Ffar2 antibody is a humanized anti-Ffar2 antibody derived from any known, or yet to be discovered, non-human anti-Ffar2 antibody.

In another embodiment of any of the aspects, the antibody or antibody reagent binds to an amino acid sequence that corresponds to the amino acid sequence encoding Ffar2 (SEQ ID NO: 1).

In another embodiment of any of the aspects, the anti-Ffar2 antibody or antibody reagent binds to an amino acid sequence that comprises the sequence of SEQ ID NO: 1; or binds to an amino acid sequence that comprises a sequence with at least 80%, at least 85%, at least 90%, at least 95%, at least 96%, at least 97%, at least 98%, at least 99% or greater sequence identity to the sequence of SEQ ID NO: 1. In one embodiment of any of the aspects, the anti-Ffar2 antibody or antibody reagent binds to an amino acid sequence that comprises the entire sequence of SEQ ID NO: 1 In another embodiment, of any of the aspects, the antibody or antibody reagent binds to an amino acid sequence that comprises a fragment of the sequence of SEQ ID NO: 1, wherein the fragment is sufficient to bind its target, e.g., Ffar2 or a metabolite that is a ligand for Ffar2, and result in the activation of Ffar2 level and/or activity. The antibody can directly or indirectly affect Ffar2 levels, e.g. by binding to a transcriptional repressor protein of Ffar2 gene expression thereby increasing gene expression of Ffar2.

In one embodiment of any of the aspects, the agent that increases Ffar2 is an antisense oligonucleotide. As used herein, an “antisense oligonucleotide” refers to a synthesized nucleic acid sequence that is complementary to a DNA or mRNA sequence, such as that of a microRNA. Antisense oligonucleotides are typically designed to block expression of a DNA or RNA target by binding to the target and halting expression at the level of transcription, translation, or splicing. Antisense oligonucleotides as described herein are complementary nucleic acid sequences designed to hybridize under cellular conditions to a gene. Thus, oligonucleotides are chosen that are sufficiently complementary to the target, i.e., that hybridize sufficiently well and with sufficient specificity in the context of the cellular environment, to give the desired effect. For example, an antisense oligonucleotide that activates or increases levels of Ffar2 directly or indirectly may comprise at least 5, at least 10, at least 15, at least 20, at least 25, at least 30, or more bases complementary to a portion of the coding sequence of the human Ffar gene (e.g., SEQ ID NO: 2), respectively. Furthermore, the antisense oligonucleotide can target transcription factors that regulate the expression of Ffar2 such as RORγt, X-box binding protein-1 (XBP1), or any other transcription factors known in the art.

In one embodiment of any of the aspects, Ffar2 is increased in the cell's genome using any genome editing system including, but not limited to, zinc finger nucleases, TALENS, meganucleases, and CRISPR/Cas systems. In one embodiment of any of the aspects, the genomic editing system used to incorporate the nucleic acid encoding one or more guide RNAs into the cell's genome is not a CRISPR/Cas system; this can prevent undesirable cell death in cells that retain a small amount of Cas enzyme/protein. It is also contemplated herein that either the Cas enzyme or the sgRNAs are each expressed under the control of a different inducible promoter, thereby allowing temporal expression of each to prevent such interference. The gene editing system can directly or indirectly modulate levels of Ffar2 expression, e.g. by inhibiting transcriptional repressors of Ffar2 that result in an increase in Ffar2 transcription.

When a nucleic acid encoding one or more sgRNAs and a nucleic acid encoding an RNA-guided endonuclease each need to be administered in vivo, the use of an adenovirus associated vector (AAV) is specifically contemplated. Other vectors for simultaneously delivering nucleic acids to both components of the genome editing/fragmentation system (e.g., sgRNAs, RNA-guided endonuclease) include lentiviral vectors, such as Epstein Barr, Human immunodeficiency virus (HIV), and hepatitis B virus (HBV). Each of the components of the RNA-guided genome editing system (e.g., sgRNA and endonuclease) can be delivered in a separate vector as known in the art or as described herein.

In one embodiment of any of the aspects, the agent activates or increases Ffar2 activity by RNA insertion or increasing RNA transcripts. Activators of the expression of a given gene can be an activating nucleic acid or transcription factor for Ffar2. In some embodiments, of any of the aspects, the inhibitory nucleic acid is an inhibitory RNA (iRNA). The RNAi can be single stranded or double stranded. The RNAi can directly or indirectly modulate Ffar2 expression, e.g. inhibiting transcriptional repressors of Ffar2 and thereby increasing Ffar2.

The iRNA can be siRNA, shRNA, endogenous microRNA (miRNA), or artificial miRNA. In one embodiment of any of the aspects, an iRNA as described herein affects inhibition of the expression and/or activity of a target, e.g. a transcriptional repressor of Ffar2. In some embodiments of any of the aspects, the agent is siRNA that inhibits transcriptional repressors of Ffar2. In some embodiments of any of the aspects, the agent is shRNA that inhibits a transcriptional repressor of Ffar2, thereby increasing Ffar2 expression.

One skilled in the art would be able to design siRNA, shRNA, or miRNA to target Ffar2 directly or indirectly by inhibiting a transcriptional repressor of Ffar2, e.g., using publically available design tools. siRNA, shRNA, or miRNA is commonly made using companies such as Dharmacon (Layfayette, CO) or Sigma Aldrich (St. Louis, Mo.).

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions

The RNA of an iRNA can be chemically modified to enhance stability or other beneficial characteristics. The nucleic acids as described herein may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference.

In another embodiment of any of the aspects, the agent is miRNA that activates or increases Ffar2 activity. MicroRNAs are small non-coding RNAs with an average length of 22 nucleotides. These molecules act by binding to complementary sequences within mRNA molecules, usually in the 3′ untranslated (3′UTR) region, thereby promoting target mRNA degradation or inhibited mRNA translation. The interaction between microRNA and mRNAs is mediated by what is known as the “seed sequence”, a 6-8-nucleotide region of the microRNA that directs sequence-specific binding to the mRNA through imperfect Watson-Crick base pairing. More than 900 microRNAs are known to be expressed in mammals. Many of these can be grouped into families on the basis of their seed sequence, thereby identifying a “cluster” of similar microRNAs. A miRNA can be expressed in a cell, e.g., as naked DNA. A miRNA can be encoded by a nucleic acid that is expressed in the cell, e.g., as naked DNA or can be encoded by a nucleic acid that is contained within a vector. The miRNA can directly or indirectly modulate Ffar2 expression. For example, the miRNA can inhibit transcriptional repressors of Ffar2.

The agent may be contained in and thus further include a vector. Many such vectors useful for transferring exogenous genes into target mammalian cells are available. The vectors may be episomal, e.g. plasmids, virus-derived vectors such cytomegalovirus, adenovirus, etc., or may be integrated into the target cell genome, through homologous recombination or random integration, e.g. retrovirus-derived vectors such as MMLV, HIV-1, ALV, etc. In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide (e.g., Ffar2 or a modulator of Ffar2) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free host cells. Another non-integrative viral vector is adenoviral vector and the adeno-associated viral (AAV) vector.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages).

Another example of a non-integrative vector is a minicircle vector. Minicircle vectors are circularized vectors in which the plasmid backbone has been released leaving only the eukaryotic promoter and cDNA(s) that are to be expressed.

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

In the various embodiments of any of the aspects, it is contemplated that variants (naturally occurring or otherwise), alleles, homologs, conservatively modified variants, and/or conservative substitution variants of any of the particular polypeptides described are encompassed. As to amino acid sequences (e.g. SEQ ID NO:1), one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ligand-mediated receptor activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments of any of the aspects, the agent is a peptide. In some embodiments of any of the aspects, the agent is a Ffar2 polypeptide. In some embodiments of any of the aspects, the agent is a vector that encodes a Ffar2 polypeptide.

In some embodiments of any of the aspects, a “peptide” or “polypeptide” as described herein (or a nucleic acid encoding such a polypeptide) can be a functional fragment of one of the amino acid sequences described herein. As used herein, a “functional fragment” is a fragment or segment of a peptide which retains at least 50% of the wild type reference polypeptide's activity according to an assay known in the art or described below herein. A functional fragment can comprise conservative substitutions of the sequences disclosed herein.

In some embodiments of any of the aspects, a polypeptide as described herein can be a variant of a polypeptide or molecule as described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 80%, at least 90%, at least 91%, at least 92%, at least 93%, at least 94%, at least 95%, at least 96%, at least 97%, at least 98%, at least 99%, identical to a native or reference sequence (e.g. SEQ ID NO: 1). The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

### Pharmaceutical Compositions:

In one aspect, described herein is a pharmaceutical composition comprising the agent described herein. In another aspect, described herein is an agent that is formulated with a pharmaceutical composition or a pharmaceutically acceptable carrier.

In another aspect, described herein is a pharmaceutical composition formulated for the treatment of a gastrointestinal disease, the pharmaceutical composition comprises: an agent that increases the level or activity of Ffar2, wherein the pharmaceutical composition increases in the number of group 3 innate lymphoid cells (ILC3s) in a subject.

In another aspect, described herein is a pharmaceutical composition formulated for the treatment of a gastrointestinal disease, the pharmaceutical composition comprises: an agent that increases the level or activity of Ffar2, a pharmaceutically acceptable carrier or excipient, wherein the pharmaceutical composition increases in the number of group 3 innate lymphoid cells (ILC3s) in a subject.

In another aspect, described herein is a pharmaceutical composition for use in the treatment of a gastrointestinal disease.

In one embodiment of any of the aspects, the pharmaceutical composition is formulated to restrict delivery of the agent to the gastrointestinal tract of the subject. In some embodiments, the pharmaceutical composition comprises an enteric coating.

In another embodiment of any of the aspects, the composition is formulated for the treatment or prevention of a gastrointestinal disease. In another embodiment, of any of the aspects, the gastrointestinal disease is a gastrointestinal infection, inflammatory bowel disease (IBD), gastrointestinal injury, appendicitis, Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer.

In one embodiment of any of the aspects, the composition further comprises a lipid vehicle. Exemplary lipid vehicles include, but are not limited to, liposomes, micelles, exosomes, lipid emulsions, and lipid-drug complex.

In another embodiment of any of the aspects, the pharmaceutical composition further comprises a particle or polymer-based vehicle. Exemplary particle or polymer-based vehicles include, but are not limited to, nanoparticles, microparticles, polymer microspheres, or polymer-drug conjugates.

As used herein, the term pharmaceutical composition” or “pharmaceutically acceptable carrier” are used interchangeably and can include any material or substance that, when combined with an active ingredient, allows the ingredient to retain biological activity and is non-reactive with the subject's immune system. Examples include, but are not limited to, any of the standard pharmaceutical carriers such as a phosphate buffered saline solution, emulsions such as oil/water emulsion, and various types of wetting agents. The term “pharmaceutically acceptable carriers” excludes tissue culture media. Non limiting examples of pharmaceutical carriers include particle or polymer-based vehicles such as nanoparticles, microparticles, polymer microspheres, or polymer-drug conjugates.

In some embodiments, the pharmaceutical composition is a liquid dosage form or solid dosage form. Liquid dosage forms for oral administration include, but are not limited to, pharmaceutically acceptable emulsions, microemulsions, solutions, suspensions, syrups and elixirs.

The liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the agent described herein is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form can also comprise buffering agents.

Solid compositions of a similar type can also be employed as fillers in soft and hard-filled gelatin capsules using such excipients as lactose or milk sugar as well as high molecular weight polyethylene glycols, and the like. The solid dosage forms of tablets, dragees, capsules, pills, can be used. Solid compositions of a similar type can also be employed as fillers in soft and hard-filled gelatin capsules using such excipients as lactose or milk sugar as well as high molecular weight polyethylene glycols, and the like. The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type can also be employed as fillers in soft and hard-filled gelatin capsules using such excipients as lactose or milk sugar as well as high molecular weight polyethylene glycols, and the like.

The agent described herein can be admixed with at least one inert diluent such as sucrose, lactose and starch. Such dosage forms can also comprise, as in normal practice, additional substances other than inert diluents, e.g., tableting lubricants and other tableting aids such as magnesium stearate and microcrystalline cellulose. In the case of capsules, tablets and pills, the dosage forms can also comprise buffering agents. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

As used herein, the term “restricts delivery of the composition to the gastrointestinal tract” refers to a formulation that permits or facilitates the delivery of the agent or pharmaceutical composition described herein to the colon, large intestine, or small intestine in viable form. Enteric coating or micro- or nano-particle formulations can facilitate such delivery as can, for example, buffer or other protective formulations.

In some embodiments, the carrier or excipient restricts delivery of the composition to the gastrointestinal tract. In some embodiments, the composition provided herein is restricted to the gastrointestinal tract by the addition of a sulfate group or a polar group to the compounds.

In some embodiments, the carrier or excipient is an enteric coating or enteric-coated drug delivery device. As used herein, the terms “enteric coating” or “enteric-coated drug delivery device” refers to any drug delivery method that can be administered orally but is not degraded or activated until the device enters the intestines. Such methods can utilize a coating or encapsulation that is degraded using e.g., pH dependent means, permitting protection of the delivery device and the agent to be administered or transplanted throughout the gastrointestinal tract until the device reaches the alkaline pH of the intestines (e.g. cecum or colon).

An enteric coating can control the location of where an agent is released in the digestive system. Thus, an enteric coating can be used such that a pharmaceutical composition does not dissolve and release the agent in the stomach, but rather travels to the intestine, where it dissolves and releases the agent in an environment that is most beneficial for increasing IL-22 secretion (e.g. from ILC3 cells). An enteric coating can be stable at low pH (such as in the stomach) and can dissolve at higher pH (for example, in the intestine). Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (trade name EmCoat 120 N, Marcoat 125); cellulose acetate phthalate (trade names AQUACOAT™, AQUACOAT ECD™, SEPIFILM™, KLUCEL™, and METOLOSE™); polyvinylacetate phthalate (trade name SURETERIC™); and methacrylic acid (trade names EUDRAGIT™, EUDRAGIT L 100-55™ from Evonik Industries, Germany).

Pharmaceutical compositions include formulations suitable for oral administration may be provided as discrete units, such as tablets, capsules, cachets, syrups, elixirs, prepared food items, microemulsions, solutions, suspensions, lozenges, or gel-coated ampules, each containing a predetermined amount of the active compound; as powders or granules; as solutions or suspensions in aqueous or non-aqueous liquids; or as oil-in-water or water-in-oil emulsions.

Accordingly, formulations suitable for rectal administration include gels, creams, lotions, aqueous or oily suspensions, dispersible powders or granules, emulsions, dissolvable solid materials, douches, and the like can be used. The formulations are preferably provided as unit-dose suppositories comprising the active ingredient in one or more solid carriers forming the suppository base, for example, cocoa butter. Suitable carriers for such formulations include petroleum jelly, lanolin, polyethyleneglycols, alcohols, and combinations thereof. Alternatively, colonic washes with the rapid recolonization deployment agent of the present disclosure can be formulated for colonic or rectal administration.

### Administration and Dosing:

The agents and pharmaceutical compositions described herein (e.g., that increases the level or activity of Ffar2) can be administered to a subject having or diagnosed as having a gastrointestinal disease. In some embodiments of any of the aspects, the methods described herein comprise administering an effective amount of an agent to a subject in order to alleviate at least one symptom of the gastrointestinal disease. As used herein, “alleviating at least one symptom of the gastrointestinal disease” is ameliorating any condition or symptom associated with the gastrointestinal disease (e.g., fatigue, pain, vomiting, nausea, upset stomach, diarrhea). As compared with an equivalent untreated control, such reduction is by at least 5%, 10%, 20%, 40%, 50%, 60%, 80%, 90%, 95%, 99% or more as measured by any standard technique. A variety of means for administering the agents and compositions described herein to subjects are known to those of skill in the art.

In one embodiment of any of the aspects, the agent is administered systemically or locally (e.g., to the gastrointestinal tract). In one embodiment of any of the aspects, the agent is administered intravenously. In one embodiment of any of the aspects, the agent is administered continuously, in intervals, or sporadically. The route of administration of the agent will be optimized for the type of agent being delivered (e.g., a small molecule), and can be determined by a skilled practitioner.

The term “effective amount” as used herein refers to the amount of an agent or composition described herein can be administered to a subject having or diagnosed as having a gastrointestinal disease needed to alleviate at least one or more symptom of the disease. The term “therapeutically effective amount” therefore refers to an amount of an agent or composition that is sufficient to provide a particular anti-gastrointestinal disease effect when administered to a typical subject. An effective amount as used herein, in various contexts, would also include an amount of an agent sufficient to delay the development of a symptom of the disease, alter the course of a symptom of the disease (e.g., slowing the progression of the gastrointestinal disease), or reverse a symptom of the disease (e.g., correcting or halting symptoms of the gastrointestinal disease). Thus, it is not generally practicable to specify an exact “effective amount”. However, for any given case, an appropriate “effective amount” can be determined by one of ordinary skill in the art using only routine experimentation.

In one embodiment of any of the aspects, the agent or composition is administered continuously (e.g., at constant levels over a period of time). Continuous administration of an agent can be achieved, e.g., by epidermal patches, continuous release formulations, or on-body injectors.

In one embodiment of any of the aspects, the agent or pharmaceutical composition is administered in intervals (e.g., at various levels over a given period of time). In some embodiments, the agent or pharmaceutical composition is administered hourly, daily, weekly, or monthly. By way of example only, the agent or pharmaceutical composition can be administered orally twice a day for a period of 1-2 weeks.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 or EC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring gastrointestinal function, or blood work, among others. The dosage can be determined by a physician and adjusted, as necessary, to suit observed effects of the treatment.

“Unit dosage form” as the term is used herein refers to a dosage for suitable one administration. By way of example a unit dosage form can be an amount of therapeutic disposed in a delivery device, e.g., a syringe or intravenous drip bag. In one embodiment of any of the aspects, a unit dosage form is administered in a single administration. In another embodiment, more than one unit dosage form can be administered simultaneously.

The dosage of the agent as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further agents, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

In one embodiment of any of the aspects, the agent or composition described herein is used as a monotherapy. In one embodiment of any of the aspects, the agents described herein can be used in combination with other known agents and therapies for a gastrointestinal disease. Administered “in combination,” as used herein, means that two (or more) different treatments are delivered to the subject during the course of the subject's affliction with the disorder, e.g., the two or more treatments are delivered after the subject has been diagnosed with the disorder (a gastrointestinal disease) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The agents described herein and the at least one additional therapy can be administered simultaneously, in the same or in separate compositions, or sequentially. For sequential administration, the agent described herein can be administered first, and the additional agent can be administered second, or the order of administration can be reversed. The agent and/or other therapeutic agents, procedures or modalities can be administered during periods of active disorder, or during a period of remission or less active disease. The agent can be administered before another treatment, concurrently with the treatment, post-treatment, or during remission of the disorder.

Therapeutics currently used to treat or prevent a gastrointestinal disease include, but are not limited to, antibiotics (e.g. aminosalicylic acid, norflaxacin, penicillin, cephalosporin), antivirals (e.g. zanamivir, oseltamivir), vaccines, corticosteroids (e.g. hydrocortisone, prednisone, prednisolone, budesonide), analgesics (e.g. acetaminophen, ibuprofen), non-steroidal anti-inflammatory drugs (e.g. mesalamine), anti-inflammatory drugs (e.g. sulfasalazine), immunosuppressants (e.g. infliximab, azathioprine, adalimumab, mercaptopurine), dietary supplements (e.g. iron), surgeries (e.g. colostomy, ileostomy, colectomy, proctocolectomy), IV fluids, enemas, other treatments for gastrointestinal disease are known in the art.

When administered in combination, the agent or composition and the additional agent (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of a gastrointestinal disease) is lower (e.g., at least 20%, at least 30%, at least 40%, or at least 50% lower) than the amount or dosage of each agent individually required to achieve the same therapeutic effect.

In some embodiments of any of the aspects, the agent is administered by direct injection, subcutaneous injection, muscular injection, oral, or nasal administration.

Parenteral dosage forms of an agents described herein can be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

In some embodiments of any of the aspects, described herein is an agent or pharmaceutical composition that is administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound of formula (I)'s onset of action, duration of action, plasma levels within the therapeutic window, and peak blood levels. In particular, controlled- or extended-release dosage forms or formulations can be used to ensure that the maximum effectiveness of an agent is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with any agent described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, DUOLITE® A568 and DUOLITE® AP143 (Rohm&Haas, Spring House, Pa. USA).

### Efficacy:

The efficacy of an agents described herein, e.g., for the treatment of a gastrointestinal disease, can be determined by the skilled practitioner. However, a treatment is considered “effective treatment,” as the term is used herein, if one or more of the signs or symptoms of the gastrointestinal disease are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., fatigue, pain, weight loss, vomiting, or nausea. Efficacy can also be measured by a failure of an individual to worsen as assessed by hospitalization, or need for medical interventions (i.e., progression of the symptoms). Methods of measuring these indicators are known to those of skill in the art and/or are described herein. Efficacy can be assessed in animal models of a condition described herein, for example, a mouse model or an appropriate animal model of gastrointestinal disease (e.g. DSS-colonic injury model or T cell transfer colitis model), as the case may be. When using an experimental animal model, efficacy of treatment is evidenced when a statistically significant change in a marker is observed, e.g., reduced diarrhea, weight gain, etc.).

The dosage administered, as single or multiple doses, to an individual will vary depending upon a variety of factors, including pharmacokinetic properties of the inhibitor, the route of administration, conditions and characteristics (sex, age, body weight, health, size) of subjects, extent of symptoms, concurrent treatments, frequency of treatment and the effect desired. A therapeutically effective amount is also one in which any toxic or detrimental effects of the therapeutic agent are outweighed by the therapeutically beneficial effects. The effective amount in each individual case can be determined empirically by a skilled artisan according to established methods in the art and without undue experimentation. In general, the phrases “therapeutically-effective” and “effective for the treatment, prevention, or inhibition”, are intended to qualify agonist as disclosed herein which will achieve the goal of reduction in the severity of a gastrointestinal disease or at one related symptom thereof.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of use or administration utilized.

The effective dose can be estimated initially from cell culture assays. A dose can be formulated in animals. Generally, the compositions are administered so that a compound of the disclosure herein is used or given at a dose from 1 μg/kg to 1000 mg/kg; 1 μg/kg to 500 mg/kg; 1 μg/kg to 150 mg/kg, 1 μg/kg to 100 mg/kg, 1 μg/kg to 50 mg/kg, 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg, 100 μg/kg to 1 mg/kg, 1 mg/kg to 100 mg/kg, 1 mg/kg to 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. It is to be understood that ranges given here include all intermediate ranges, for example, the range 1 mg/kg to 10 mg/kg includes 1 mg/kg to 2 mg/kg, 1 mg/kg to 3 mg/kg, 1 mg/kg to 4 mg/kg, 1 mg/kg to 5 mg/kg, 1 mg/kg to 6 mg/kg, 1 mg/kg to 7 mg/kg, 1 mg/kg to 8 mg/kg, 1 mg/kg to 9 mg/kg, 2 mg/kg to 10 mg/kg, 3 mg/kg to 10 mg/kg, 4 mg/kg to 10 mg/kg, 5 mg/kg to 10 mg/kg, 6 mg/kg to 10 mg/kg, 7 mg/kg to 10 mg/kg, 8 mg/kg to 10 mg/kg, 9 mg/kg to 10 mg/kg, and the like. Further contemplated is a dose (either as a bolus or continuous infusion) of about 0.1 mg/kg to about 10 mg/kg, about 0.3 mg/kg to about 5 mg/kg, or 0.5 mg/kg to about 3 mg/kg. It is to be further understood that the ranges intermediate to those given above are also within the scope of this disclosure, for example, in the range 1 mg/kg to 10 mg/kg, for example use or dose ranges such as 2 mg/kg to 8 mg/kg, 3 mg/kg to 7 mg/kg, 4 mg/kg to 6 mg/kg, and the like.

The agent described herein can be administered at once, or can be divided into a number of smaller doses to be administered at intervals of time. It is understood that the precise dosage and duration of treatment will be a function of the location of where the composition is parenterally administered, the carrier and other variables that can be determined empirically using known testing protocols or by extrapolation from in vivo or in vitro test data. It is to be noted that concentrations and dosage values can also vary with the age of the individual treated. It is to be further understood that for any particular subject, specific dosage regimens can need to be adjusted over time according to the individual need and the professional judgment of the person administering or supervising the administration of the formulations. Hence, the concentration ranges set forth herein are intended to be exemplary and are not intended to limit the scope or practice of the claimed formulations.

### Assays, Treatments, and Diagnostics for a Gastrointestinal Disease:

In one aspect of any of the embodiments, described herein is a method of treating a gastrointestinal disease in a subject, the method comprises: (a) measuring the level of Ffar2 in a biological sample of a subject; and (b) comparing the measurement of (a) to a reference level; (c) identifying a subject with decreased Ffar2 in (a) as compared to a reference level as having a gastrointestinal disease; and (d) administering to the subject having a gastrointestinal disease an agent that modulates Ffar2.

In another aspect, described herein is a method of treating a gastrointestinal disease in a subject, the method comprises: (a) receiving the results of an assay that indicates that the level of Ffar2 in a biological sample of a subject is decreased compared to a reference level; and (b) administering to the subject an agent that modulates Ffar2.

In one embodiment of any of the aspects, the gastrointestinal disease is selected from the group consisting of: a gastrointestinal infection, inflammatory bowel disease (IBD), gastrointestinal injury, appendicitis, Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer.

In another embodiment of any of the aspects, the method further comprises, prior to step (a), obtaining a biological sample from the subject. The biological sample can be obtained by methods known in the art such as blood draw or surgical methods. In another embodiment, of any of the aspects, the biological sample is a blood sample, buffy coat, serum, or tissue.

In another embodiment, of any of the aspects, the tissue is removed from the esophagus, small intestine, large intestine, or colon. In another embodiment, of any of the aspects, the tissue is a colonic solitary lymphoid tissue. Surgical removal of intestinal tissues is standard in the medical profession and methods are known in the art. For example, a colectomy, is a procedure in which part of the colon or a tissue sample from the colon is removed. Accordingly, if a gastrointestinal injury occurs, due to a car accident, military wounds, dextran sulfate sodium (DSS) injury, etc., tissue from the gastrointestinal tract can be repaired and a small biopsy can be removed for testing.

In another aspect, described herein is an assay for identifying an agent that modulates a functional property of an immune lymphoid cell, the assay comprises (a) contacting a population of innate lymphoid cells with an agent; and (b) detecting the level of Ffar2 wherein detecting a change in Ffar2 levels after contacting step (a) identifies the agent as one that can modulate a functional property of innate lymphoid cells.

In one embodiment of any of the aspects, the detecting step further comprises detecting the level of IL-22. In another embodiment, of any of the aspects, the detecting step further comprises detecting the level of RORγt. In another embodiment, of any of the aspects, the detecting step further comprises detecting the level of phosphorylated-Akt, XBP1, phosphorylated STAT3, phosphorylated ERK, mucin 2, mucin 3, mucin 4, mucin 5a, mucin 5b, Regenerating islet-derived protein (Reg) 3 alpha, Reg 3 beta, Reg 3 gamma, AhR, CCR6, Ki-67, GATA3, NKp46, pAKT, pp38, pERK, pSTAT3, IL-17, and/or Foxp3.

In another embodiment of any of the aspects, the detecting is accomplished by RT-PCR, flow cytometry, immunohistochemistry, Western Blot, enzyme-linked immunosorbent assay (ELISA), mass spectrometry, or microscopy. These methods are well known in the art.

For the assay, cells can be optionally allowed to grow for a period time before contacting with the agent. In some embodiments, a practitioner can obtain cells (e.g. ILC3s) that are already plated in the appropriate vessel and allowed to grow for a period of time. In other embodiments, the practitioner plates the cell in the appropriate vessel and allow the cells to grow for a period time, e.g., at least one day, at least two days, at least three days, at least four days, at least five days, at least six days, at least seven days or more before contacting with the test compound.

After the agent or test compound has been in contact with the cell or population of cells (e.g. ILC3s) for a sufficient period of time, amount of reporter (e.g., expression or activity) is measured and compared to a control or reference. For example, contact time can be from seconds to days or weeks. The practitioner can optimize the contact time for obtaining an optimal signal-to-noise ratio, time constraints, amount of test compound to be tested, number of cells, test volume, availability of reagents for the assay, and the like.

As used herein, the term “test compound” refers to compounds, agents as described herein, and/or pharmaceutical compositions that are to be screened for their ability to stimulate and/or increase and/or promote Ffar2 activity or expansion of ILC3 populations. The test compounds can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; an extract made from biological materials such as bacteria, plants, fungi, or animal cells; animal tissues; naturally occurring or synthetic compositions; and any combinations thereof. In some embodiments of any of the aspects, the agent or test compound is a small molecule. In some embodiments of any of the aspects, the agent or test compound is a SCFA.

The number of possible test compounds runs into millions. Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule, Pharmacopia, Graffinity, Panvera, Vitas-M Lab, Biomol International and Oxford. These libraries can be screened using the screening devices and methods described herein. Chemical compound libraries such as those from NIH Roadmap, Molecular Libraries Screening Centers Network (MLSCN) can also be used. A comprehensive list of compound libraries can be found on the world-wide web at http://<broad.harvard.edu/chembio/platform/screening/compound_libraries/index.htm>. A chemical library or compound library is a collection of stored chemicals usually used ultimately in high-throughput screening or industrial manufacture. The chemical library can consist in simple terms of a series of stored chemicals. Each chemical has associated information stored in some kind of database with information such as the chemical structure, purity, quantity, and physiochemical characteristics of the compound.

Depending upon the particular embodiment being practiced, the test compounds can be provided free in solution, or may be attached to a carrier, or a solid support, e.g., beads. A number of suitable solid supports may be employed for immobilization of the test compounds. Examples of suitable solid supports include agarose, cellulose, dextran (commercially available as, i.e., Sephadex™, Sepharose™) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group.

The test compound can be tested at any desired concentration. For example, the test compound can be tested at a final concentration of from 0.01 nanomolar to about 10 millimolar. Further, the test can be tested at 2 or more (e.g., 2, 3, 4, 5, 6, 7, 8, 9, 10 or more) different concentrations. This can be helpful if the test compound is active only in a range of concentration. When the test compound is tested at 2 or more different concentrations, the concentration difference can range from 10-10,000 fold (e.g., 10-5000 fold, 10-1000 fold, 10-500 fold, or 10-250 fold).

In some embodiments of any of the aspects, the agent or test compound is assayed more than once and selected if it reproducibly modulates Ffar2 expression or activity.

In some embodiments of any of the aspects, the assay further comprises the step of determining of the compound has scored on any other screens. This can be accomplished by looking at the various chemical databases that describe activity of compounds in various assays. This can help in identifying compounds that are unique to the present assay.

In some embodiments of any of the aspects, the selected test compound exhibits dose-dependent modulation of Ffar2 expression or activity. In some embodiments of any of the aspects, selected test compound exhibits maximal modulation of Ffar2 expression or activity in the assay. This can be helpful because some highly potent modulators (based on EC50) can yield only weak maximal activation, whereas other less potent modulators (based on EC50) can produce significantly greater activation, even at doses below their EC50.

The assay can be performed any suitable container or apparatus available to one of skill in the art for cell culturing. For example, the assay can be performed in 24-, 96-, or 384-well plates. In one embodiment of any of the aspects, the assay is performed in a 384-well plate.

Cells for the aspects disclosed herein can be obtained from any source available to one of skill in the art. Additionally, cells can be of any origin. Accordingly, in some embodiments, the cell is from a mammalian source. In some embodiments, of any of the aspects, the cells are innate lymphoid cells, or group 3 innate lymphoid cells (ILC3s). In another embodiment, of any of the aspects, the innate lymphoid cells are CCR6 positive or CCR6 negative cells.

In some embodiments of any of the aspects, the cell is from a subject, e.g., a patient. In some embodiments of any of the aspects, the cell is from the esophagus, small intestine, large intestine, or colon of the subject. In some embodiments of any of the aspects, the subject, is a patient in need of treatment for a gastrointestinal disease.

Some embodiments of the various aspects described herein can be described as in the following numbered paragraphs:
1) A method for treating or preventing a gastrointestinal disease, the method comprising: administering to a subject in need thereof an agent that increases the level or activity of Free Fatty Acid Receptor 2 (Ffar2) in the subject.
2) The method of paragraph 1, wherein the agent preferentially binds to a Ffar2 receptor.
3) The method of any one of paragraphs 1-2, wherein the agent induces an increase in the number of group 3 innate lymphoid cells (ILC3s).
4) The method of any one of paragraphs 1-3, wherein the agent induces secretion of interleukin-22 (IL-22) and/or interleukin-17 (IL-17) from ILC3s.
5) The method of any one of paragraphs 1-4, wherein the agent is selected from the group consisting of a small molecule, an antibody, a peptide, a genome editing system, a vector, and a nucleic acid.
6) The method of paragraph 5, wherein the small molecule is a short chain fatty acid (SCFA) pharmaceutically acceptable salt, or derivative thereof
7) The method of paragraph 6, wherein the SCFA is selected from the group consisting of: propionic acid, acetic acid, butyric acid, formic acid, isobutyric acid, valeric acid, isovaleric acid, formate, acetate, propionate, butyrate, pentanoate, isobutyrate, valerate, isovalerate, sodium propionate, and pharmaceutically acceptable salts thereof.
8) The method of paragraph 5, wherein the small molecule is selected from the group consisting of: ES43012-SOD, trans-2-methylcrotonic acid, propiolic acid, angelic acid, compound 34, sodium acetate, sodium propionate, sodium butyrate, formate, pentanoate, (S)-2-(4-chlorophenyl)-3,3-dimethyl-N-(5-phenylthiazol-2-yl)butanamide, BTI-A-404, BTI-A-292, AZ1729, and any derivative thereof
9) The method of paragraph 5, wherein the vector or nucleic acid that encodes an Ffar2 polypeptide.
10) The method of paragraph 9, wherein the vector is non-integrative or integrative.
11) The method of paragraph 10, wherein the non-integrative vector is selected from the group consisting of an episomal vector, an EBNA1 vector, a minicircle vector, a non-integrative adenovirus, a non-integrative RNA, and a Sendai virus.
12) The method of any one of paragraphs 5, 9, or 10, wherein the vector is an episomal vector.
13) The method of any one of paragraphs 5, 9, or 10, wherein the vector is a lentiviral vector.
14) The method of any one of paragraphs 1-13, wherein the agent is formulated with a pharmaceutical composition.
15) The method of paragraph 14, wherein the pharmaceutical composition is formulated to restrict delivery of the agent to the gastrointestinal tract of the subject.
16) The method of any one of paragraphs 14-15, wherein the pharmaceutical composition comprises an enteric coating.
17) The method of any one of paragraphs 1-16, wherein the gastrointestinal disease is selected from the group consisting of: a gastrointestinal infection, inflammatory bowel disease (IBD), gastrointestinal injury, appendicitis, Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer.
18) The method of any one of paragraphs 1-17, wherein the administering reduces inflammation of the gastrointestinal tract.
19) The method of any one of paragraphs 1-18, wherein the agent is administered by direct injection, subcutaneous injection, muscular injection, oral, or nasal administration.
20) The method any one of paragraphs 1-19, wherein the subject is a mammal.
21) The method any one of paragraphs 1-20, wherein the subject is a human.
22) The method any one of paragraphs 1-21, wherein the level or activity of Ffar2 is increased by at least 50%, at least 60%, at least 70%, at least 80%, at least 90%, or more as compared to an appropriate control.
23) The method of any one of paragraphs 1-22, wherein the secretion of IL-22 and/or IL-17 from ILC3s is increased by at least 50%, at least 60%, at least 70%, at least 80%, at least 90%, or more as compared to an appropriate control.
24) A method of reducing inflammation in the gastrointestinal tract of a subject, the method comprising: administering to a subject an agent that increases the level or activity of Free Fatty Acid Receptor 2 (Ffar2) in the subject.
25) The method of paragraph 24, wherein the agent preferentially binds to a Ffar2 receptor.
26) The method of any one of paragraphs 24-25, wherein the agent induces an increase in the number of group 3 innate lymphoid cells (ILC3s).
27) The method of any one of paragraphs 24-26, wherein the agent induces secretion of interleukin-22 (IL-22) and/or interleukin-17 (IL-17) from ILC3s.
28) The method of any one of paragraphs 24-27, wherein the agent is selected from the group consisting of a small molecule, an antibody, a peptide, a genome editing system, a vector, and a nucleic acid.
29) The method of paragraph 28, wherein the small molecule is a short chain fatty acid (SCFA) pharmaceutically acceptable salt, or derivative thereof
30) The method of paragraph 29, wherein the SCFA is selected from the group consisting of: propionic acid, acetic acid, butyric acid, formic acid, isobutyric acid, valeric acid, isovaleric acid, formate, acetate, propionate, butyrate, pentanoate, isobutyrate, valerate, isovalerate, sodium propionate, and pharmaceutically acceptable salts thereof.
31) The method of paragraph 28, wherein the small molecule is selected from the group consisting of: ES43012-SOD, trans-2-methylcrotonic acid, propiolic acid, angelic acid, compound 34, sodium acetate, sodium propionate, sodium butyrate, formate, pentanoate, (S)-2-(4-chlorophenyl)-3,3-dimethyl-N-(5-phenylthiazol-2-yl)butanamide, BTI-A-404, BTI-A-292, AZ1729, and any derivative thereof
32) The method of paragraph 28, wherein the vector or nucleic acid that encodes an Ffar2 polypeptide.
33) The method of any one of paragraphs 24-31, wherein the agent is formulated with a pharmaceutical composition.
34) The method of paragraph 33, wherein the pharmaceutical composition is formulated to restrict delivery of the agent to the gastrointestinal tract of the subject.
35) The method of any one of paragraphs 33-34, wherein the pharmaceutical composition comprises an enteric coating.
36) An assay for identifying an agent that modulates a functional property of an immune lymphoid cell, the assay comprising:
   a. contacting a population of innate lymphoid cells with an agent; and
   b. detecting the level of Ffar2
wherein detecting a change in Ffar2 levels after contacting step (a) identifies the agent as one that can modulate a functional property of innate lymphoid cells.
37) The assay of paragraph 36, wherein detecting step (b) further comprises detecting the level of IL-22.
38) The assay of any one of paragraphs 36-37, wherein detecting step (b) further comprises detecting the level of RORγt, X-box binding protein-1 (XBP1), phosphorylated-Akt, phosphorylated STAT3, phosphorylated ERK, mucin 2, mucin 3, mucin 4, mucin 5a, mucin 5b, Regenerating islet-derived protein (Reg) 3 alpha, Reg 3 beta, Reg 3 gamma, and/or Ki-67.
39) The assay of any one of paragraphs 36-38, wherein the innate lymphoid cells are group 3 innate lymphoid cells (ILC3).
40) A method of treating a gastrointestinal disease in a subject, the method comprising:
   a. measuring the level of Ffar2 in a biological sample of a subject; and
   b. comparing the measurement of (a) to a reference level;
   c. identifying a subject with decreased Ffar2 in (a) as compared to a reference level as having a gastrointestinal disease; and
   d. administering to the subject having a gastrointestinal disease an agent that modulates Ffar2.
41) The method of paragraph 40, wherein the gastrointestinal disease is selected from the group consisting of: a gastrointestinal infection, inflammatory bowel disease (IBD), gastrointestinal injury, appendicitis, Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer.
42) The method of any one of paragraphs 40-41, further comprising, prior to (a), obtaining a biological sample from the subject.
43) The method of any one of paragraphs 40-42, wherein the biological sample is a blood sample, buffy coat, serum, or tissue.
44) The method of any one of paragraphs 40-43, wherein the tissue is removed from the esophagus, small intestine, large intestine, or colon.
45) A pharmaceutical composition formulated for the treatment of a gastrointestinal disease, the pharmaceutical composition comprising:
   an agent that increases the level or activity of Ffar2,
   wherein the pharmaceutical composition increases in the number of group 3 innate lymphoid cells (ILC3s) in a subject.
46) The pharmaceutical composition of paragraph 45, wherein the agent is selected from the group consisting of a small molecule, an antibody, a peptide, a genome editing system, a vector, and a nucleic acid.
47) The pharmaceutical composition of paragraph 46, wherein the small molecule is a short chain fatty acid (SCFA) pharmaceutically acceptable salt, or derivative thereof
48) The pharmaceutical composition of paragraph 47, wherein the SCFA is selected from the group consisting of: propionic acid, acetic acid, butyric acid, formic acid, isobutyric acid, valeric acid, isovaleric acid, formate, acetate, propionate, butyrate, pentanoate, isobutyrate, valerate, isovalerate, sodium propionate, and pharmaceutically acceptable salts thereof
49) The pharmaceutical composition of paragraph 46, wherein the small molecule is selected from the group consisting of: ES43012-SOD, trans-2-methylcrotonic acid, propiolic acid, angelic acid, compound 34, sodium acetate, sodium propionate, sodium butyrate, formate, pentanoate, (S)-2-(4-chlorophenyl)-3,3-dimethyl-N-(5-phenylthiazol-2-yl)butanamide, BTI-A-404, BTI-A-292, AZ1729, and any derivative thereof
50) The pharmaceutical composition of paragraph 46, wherein the vector or nucleic acid encodes an Ffar2 polypeptide.
51) The pharmaceutical composition of any one of paragraphs 45-50, wherein the pharmaceutical composition is formulated to restrict delivery of the agent to the gastrointestinal tract of the subject.
52) The pharmaceutical composition of any one of paragraphs 45-51, wherein the pharmaceutical composition comprises an enteric coating.

### EXAMPLES

### Example 1: Metabolite-Sensing Receptor Ffar2 Regulates Colonic Group 3 Innate Lymphoid Cells and Gut Immunity

### Summary

Group 3 innate lymphoid cells (ILC3s) sense environmental signals that and play a role in gut homeostasis and host defense. However, metabolite-sensing G-protein-coupled receptors that regulate colonic ILC3s remain poorly understood. Results show that colonic ILC3s expressed Ffar2, a microbial metabolite-sensing receptor, and that Ffar2 agonism promoted ILC3 expansion. Deletion of Ffar2 in ILC3s decreased in situ proliferation and ILC3-derived IL-22 production. Ffar2 agonism of CCR6+ ILC3s enhanced their proliferation and IL-22 production and influenced ILC3 expansion in colonic lymphoid tissues. Ffar2 agonism activated AKT signaling and increased ILC3-derived IL22 via an AKT and STAT3 axis. Ffar2 deletion in ILC3s led to impaired gut epithelial function: altering mucus-associated proteins and antimicrobial peptides and increased susceptibility to colonic injury and inflammation. These findings demonstrate that Ffar2 regulates colonic ILC3 proliferation and function in a cell-intrinsic manner and identifies an ILC3-receptor signaling pathway regulating gut inflammatory tone and pathogen defense.

### Introduction

The innate lymphoid cell (ILC) family plays significant roles in immunity, inflammation, and tissue homeostasis and repair. ILCs are classified into three groups on the basis of signature transcription factors and distinct effector cytokines: Group 1 ILCs (ILC1s) require T-bet and produce interferon-γ (IFN-γ), Group 2 ILCs (ILC2s) express GATA3 and produce the type 2 cytokines interleukin 5 (IL-5) and IL-13, and Group 3 ILCs (ILC3s) are a heterogeneous population expressing transcription factor RAR-related orphan receptor gamma t (RORγt) and have the ability to produce IL-22 and/or IL-17^{1, 2, 3}.

ILC3s are enriched in the intestine, where they maintain gut homeostasis by orchestrating: lymphoid organ development, containment of commensal bacterial, tissue repair, host defense and regulation of adaptive immunity^{2, 4, 5, 6, 7} ILC3s can be divided into two subsets based on C-C chemokine receptor type 6 (CCR6) expression^{8, 9, 10, 11}. Both CCR6⁺ ILC3s and CCR6⁻ ILC3s produce IL-22, a key cytokine that is essential for recovery from tissue damage and protection against intracellular bacteria such as Citrobacter rodentium^{12, 13, 14}. CCR6⁺ ILC3s comprise lymphoid-tissue-inducer cells (LTi) and LTi-like cells. In addition to secreting IL-22, CCR6⁺ ILC3s can produce IL-17, a cytokine necessary for resistance against extracellular bacteria and fungi¹⁵. CCR6⁻ ILC3s can express NKp46 and NKp46⁺ ILC3s express T-bet and can secrete IFN-γ. CCR6⁻ NKp46⁻ ILC3s have the capacity to differentiate into NKp46⁺ ILC3s⁹. CCR6⁺ ILC3s are mainly clustered in aggregates together with stromal cells, dendritic cells (DCs) and B cells in cryptopatches, immature or mature isolated lymphoid follicles in the small intestine¹⁶. In contrast, CCR6⁻ ILC3s are scattered throughout the intestine. The steady-state adult colon has colonic patches and colonic solitary lymphoid tissues (SILTs), which are similar to small intestine lymphoid tissues^{17, 18}. Colonic patches are composed of B and T cells, segregated into clearly distinct compartments, and CCR6⁺LTi cells persist at the border of these compartments; Colonic SILTs appear in different developmental/maturation stages and contain CCR6⁻LTi cells, which are surrounded by B cell, DCs and a few T cells¹⁷.

Unlike myeloid cell population, ILC3s do not appear to express toll-like receptors or other canonical microbial-associated molecular pattern receptors. Rather, ILC3s process signals from other cells and soluble mediators within their local tissue microenvironment¹⁹. Recent studies suggest that environmental cues, such as microbial, dietary, and neuronal signals, regulate ILC3s through cell-intrinsic receptors. Bacterial metabolites and dietary components engage the aryl hydrocarbon receptor (AHR) and promote ILC3 proliferation and cytokine secretion^{20, 21, 22}. Retinoic acid (RA) and RA receptors (RARs) enhance IL-22 by ILC3s²³. Glial-derived neurotropic factor family ligand (GFL) also controls ILC3 via neuroregulatory receptor (RET) signaling²⁴.

Of the hundreds of bacterial metabolites in the gut^{25, 26}, short-chain fatty acids (SCFAs) have emerged as substantial regulators of immune responses in the gut and systemically^{27, 28, 29, 30.} SCFAs, which are produced in the colon through bacterial fermentation of dietary fiber can engage ‘metabolite-sensing’ G-protein-coupled receptors (GPCRs)^{26, 31, 32, 33}. Ffar2, a SCFA-sensing GPCR, is broadly immunomodulatory and plays a useful role in gut homeostasis and regulation of inflammation^{26, 32, 33}. Loss of Ffar2 in mice attenuates inflammation in colitis and arthritis mouse models via regulation of leukocyte chemotaxis³⁴. Ffar2 also mediates colonic Treg cell expansion and protects against T-cell transfer colitis³⁵. Ffar2 may even protect against type 1 diabetes via modulating Treg cell frequency³⁶. However, whether Ffar2 signaling regulates colonic ILC3s remains unknown.

Provided herein are results that show that colonic ILC3s express Ffar2 transcripts and Ffar2 agonism selectively promotes colonic ILC3 population frequency and number suggesting that colonic ILC3s express functional Ffar2. Ablation of Ffar2 in ILC3s decreased colonic ILC3 in situ proliferation and ILC3-derived IL-22 production. Ffar2-expressing CCR6⁺ ILC3s enhanced in situ proliferation and IL-22 production, and influenced an accumulation of ILC3s in colonic lymphoid tissues. In addition, Ffar2 signaling specifically activated AKT downstream of Ffar2 and directly regulated colonic ILC3-derived IL-22 via AKT and STAT3. Furthermore, Ffar2-deficient ILC3s led to impaired gut epithelial function by altering expression of mucus-associated proteins and antimicrobial peptides. Consequently, mice that have Ffar2-deficient ILC3s were more susceptible to both dextran sulfate sodium-induced colonic injury and C. rodentium infection. These results demonstrate that Ffar2 signaling affects colonic ILC3 proliferation and function in a cell-intrinsic manner and modulates ILC3 regulation of gut inflammatory tone and pathogen defense.

### Results

### Ffar2 Agonism Selectively Promotes Colonic ILC3 Expansion

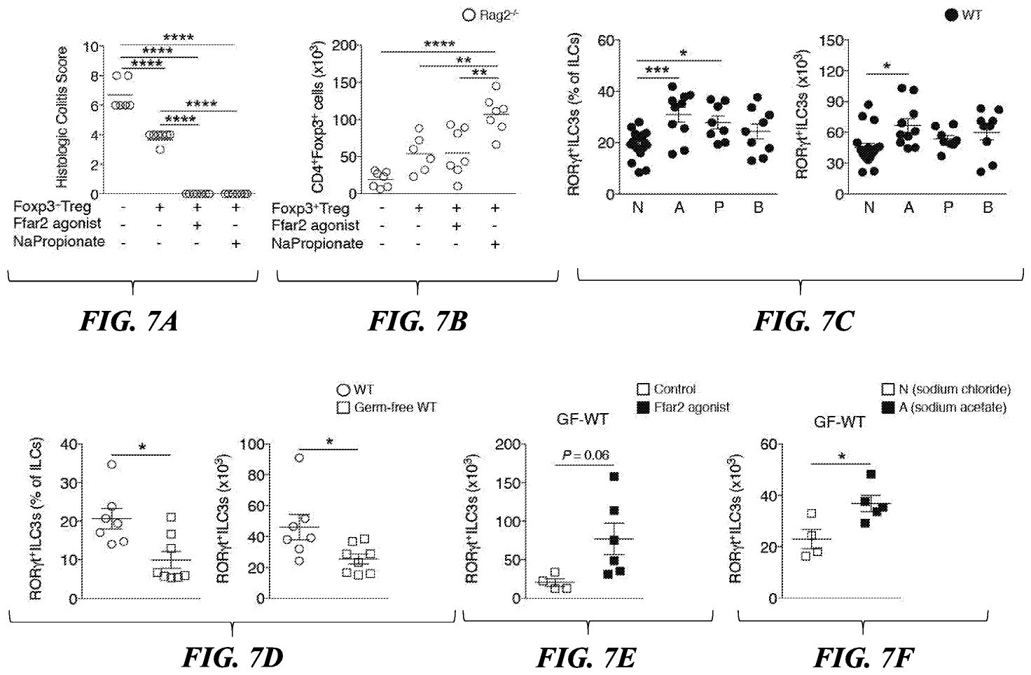

Ffar2 regulates colonic Treg cell expansion and that Ffar2 expression in Treg cells in conjunction with SCFA supplementation conferred protection against T cell-transfer colitis³⁵. To test the therapeutic potential of Ffar2 agonism for inflammatory bowel disease in a preclinical model, a Ffar2 agonist³⁷ and examined its effects in the T cell-transfer colitis model. Although Ffar2 agonism significantly ameliorated colitis similar to the effect of sodium propionate, the Ffar2 agonist did not increase colonic Foxp3⁺Treg cell number (FIG. 7A-7B) in contrast to previously published results with short chain fatty acids³⁵. This unexpected result prompted an investigation to determine if a cell population present in Rag2^{-/-} mice receiving the transferred T cells, and thus an innate immune cell population, was required for Ffar2 agonism's effects on colitis mitigation and Foxp3⁺Treg cell population enhancement.

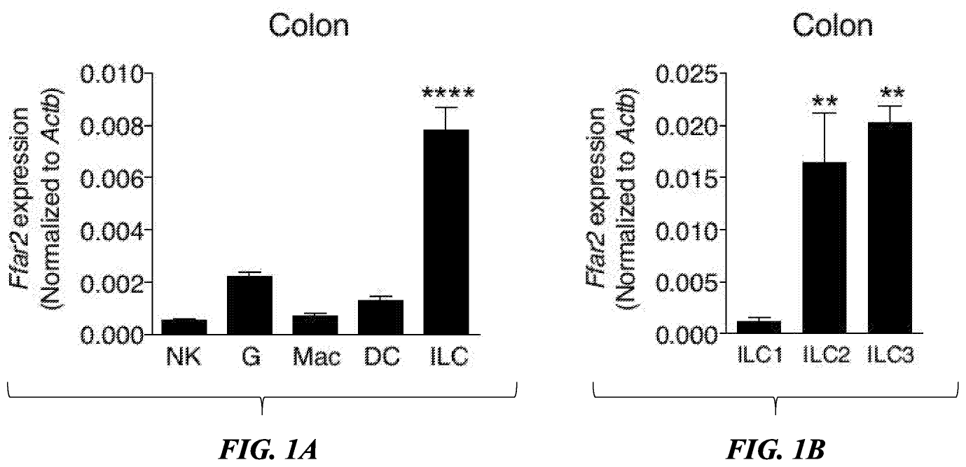

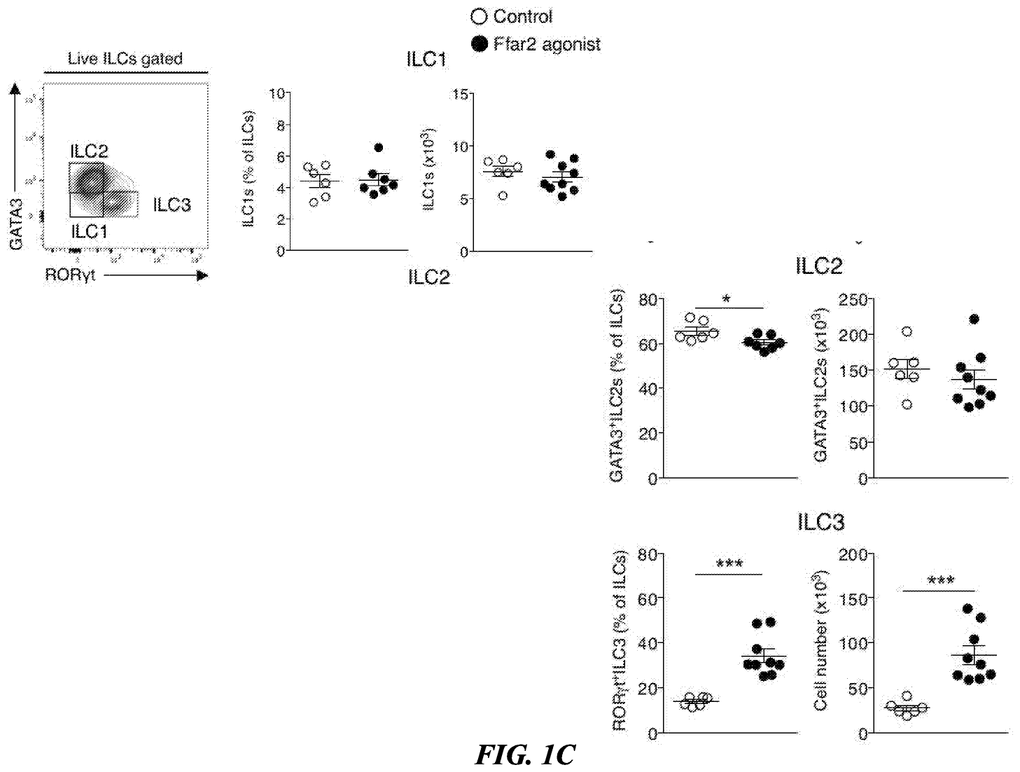

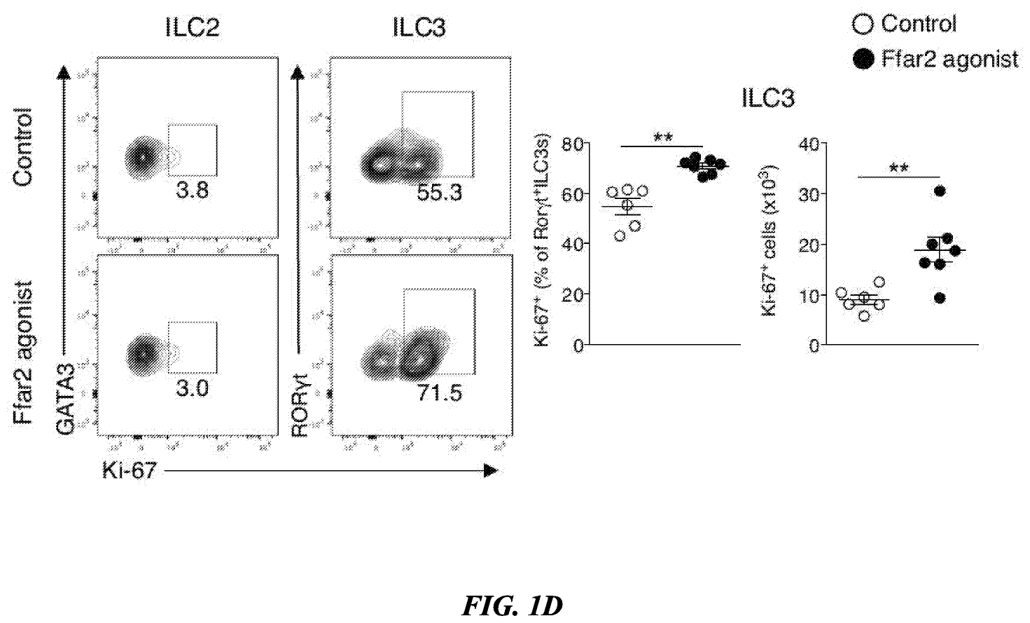

To investigate whether a colonic innate immune cell population is a target of Ffar2 agonism, Ffar2 expression levels were profiled in colonic innate immune populations and found that colonic innate lymphoid cells (ILCs) expressed higher levels of Ffar2 as compared to myeloid cells and granulocytes, which are known to express Ffar2^{34, 35} (FIG. 1A). Among colonic ILC populations, both ILC2s and ILC3s expressed significantly higher levels of Ffar2 compared to ILC1s (FIG. 1B). To determine whether colonic ILCs express functional Ffar2 on the cell surface, WT mice were fed Ffar2 agonist and analyzed colonic ILC populations. As expected, the Ffar2 agonist did not alter colonic ILC1 frequency or numbers (FIG. 1C). However, the Ffar2 agonist did increase RORγt⁺ ILC3 frequency and cell number, whereas GATA3⁺ ILC2s decreased in frequency but not in number (FIG. 1C). These data led to the experimental question of whether Ffar2 agonism selectively decreases ILC2s or increases ILC3s. To address this question, the expression of the proliferation marker Ki-67 was examined in colonic GATA3⁺ ILC2s and RORγt⁺ ILC3s from WT mice fed with the Ffar2 agonist. Colonic GATA3⁺ ILC2s exhibited limited Ki-67 expression and Ffar2 agonism did not alter the frequency of Ki-67-expressing ILC2s (FIG. 1D). In contrast, colonic RORγt⁺ ILC3s proliferated during steady state and Ffar2 agonism increased the frequency and number of Ki-67-expressing ILC3s (FIG. 1D).

Recently, butyrate has been reported to suppress Peyer's patch ILC3s³⁸. Acetate, propionate and butyrate are the three most abundant SCFAs in the colon³⁹ and these SCFAs differ in their agonism of Ffar2, with acetate and propionate functioning as far more potent activators of Ffar2⁴⁰. To confirm whether Ffar2 agonism by SCFAs regulates colonic ILC3s, mice were fed SCFAs or sodium chloride as a control and colonic ILC3s were analyzed. Sodium acetate and sodium propionate increased colonic ILC3 frequency and number, whereas sodium butyrate did not change colonic ILC3s (FIG. 7C). Thus, these data suggest that colonic ILC3s express functional Ffar2 and Ffar2 agonism selectively increases colonic RORγt⁺ ILC3s.

### Ffar2 Regulates Colonic ILC3 Proliferation and ILC3-Derived IL-22 Production

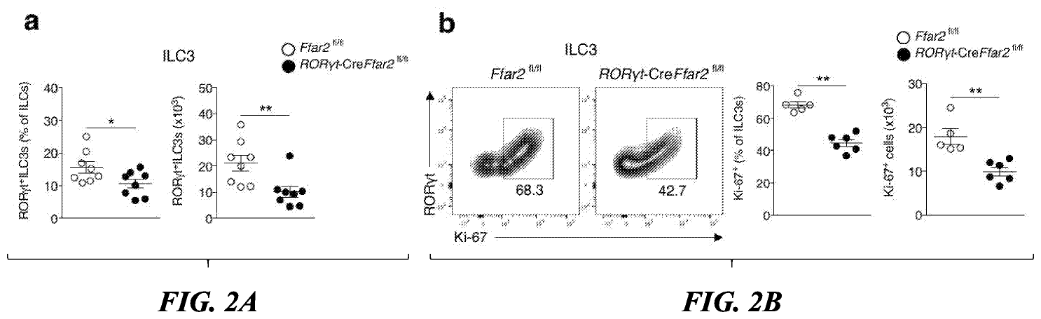

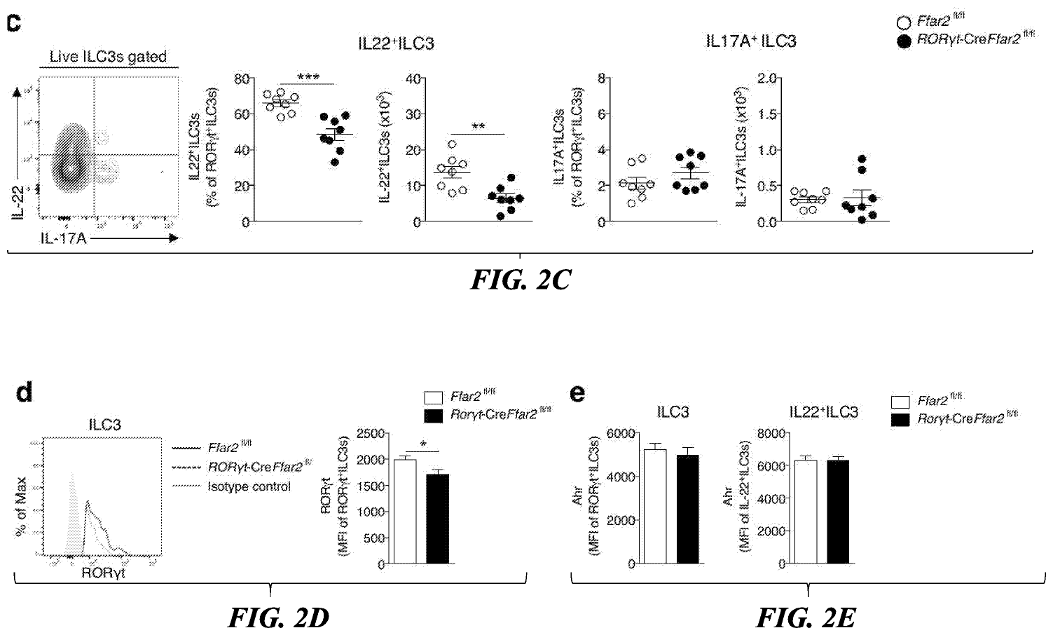

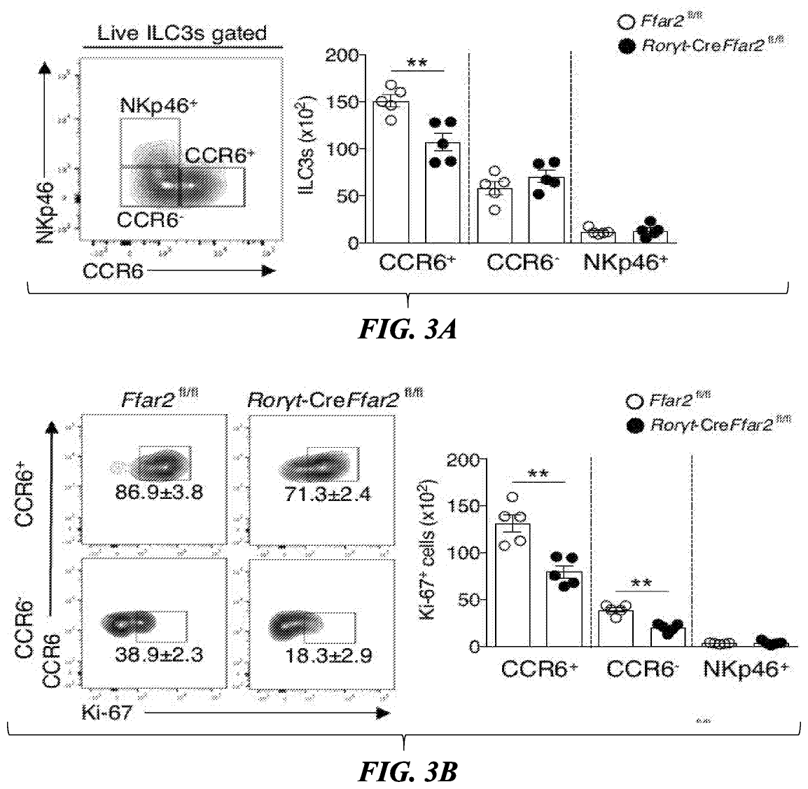

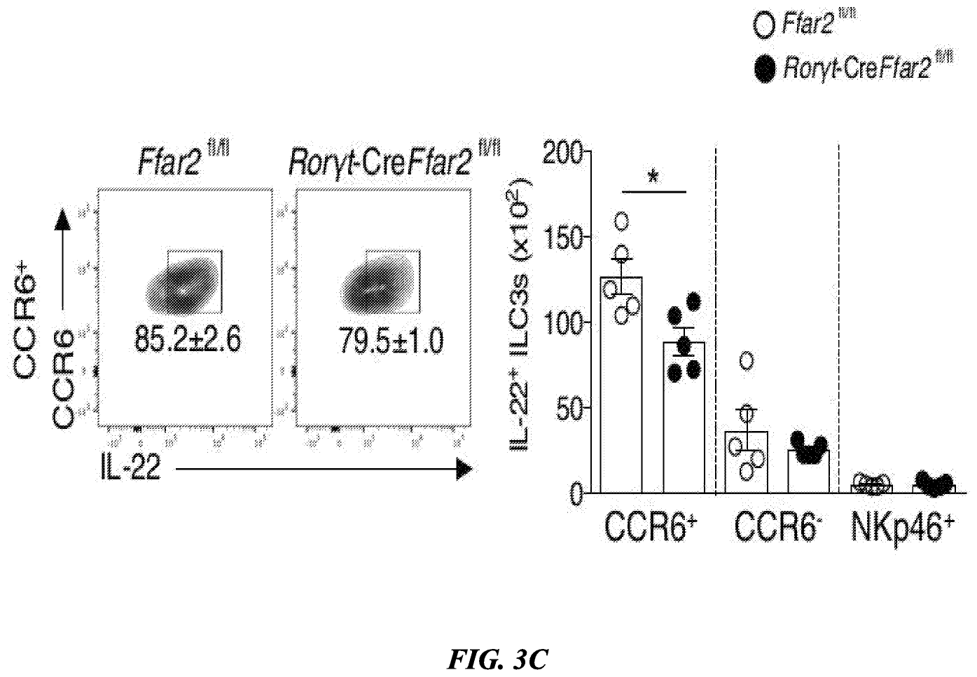

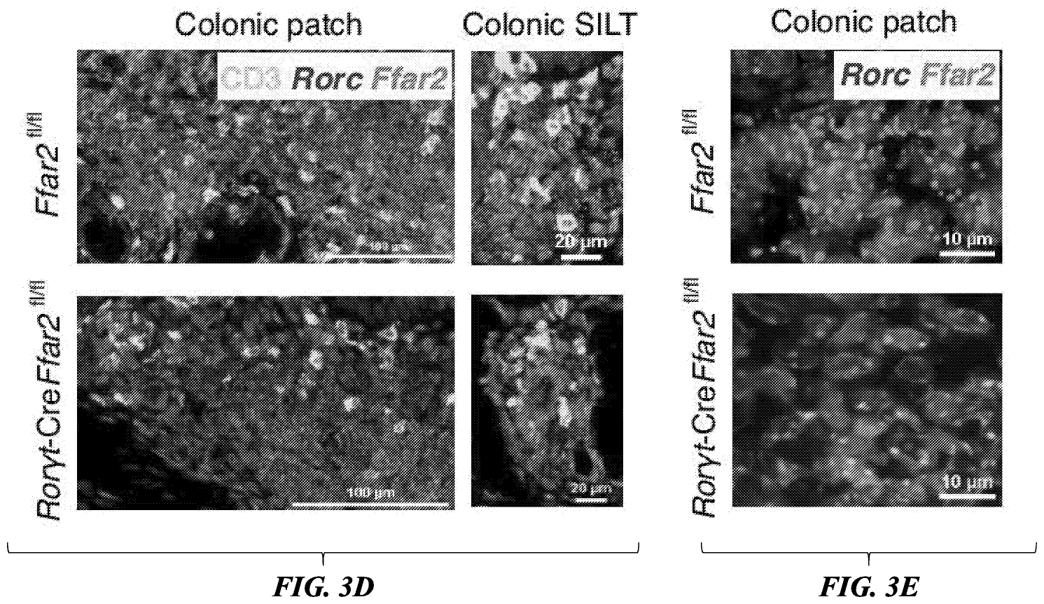

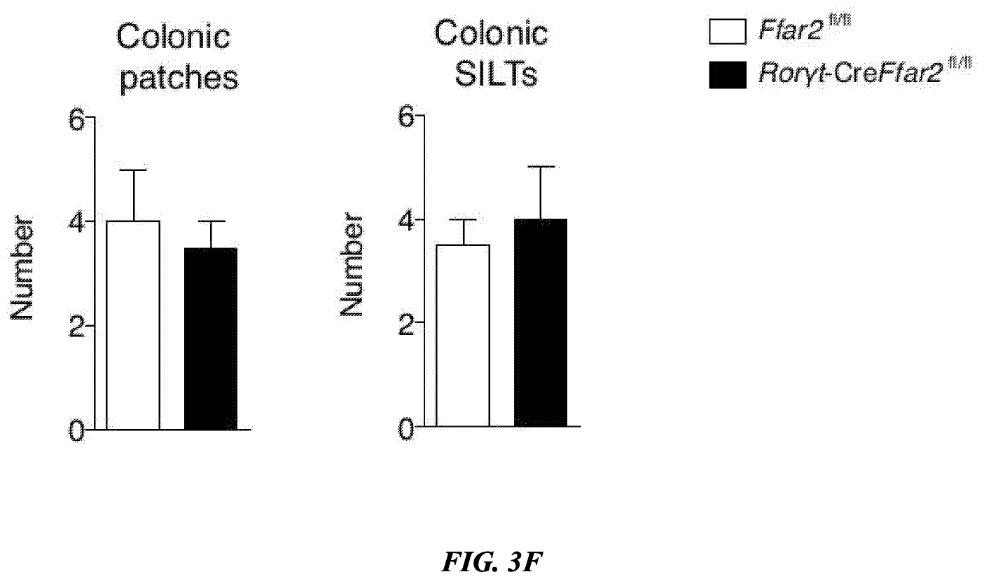

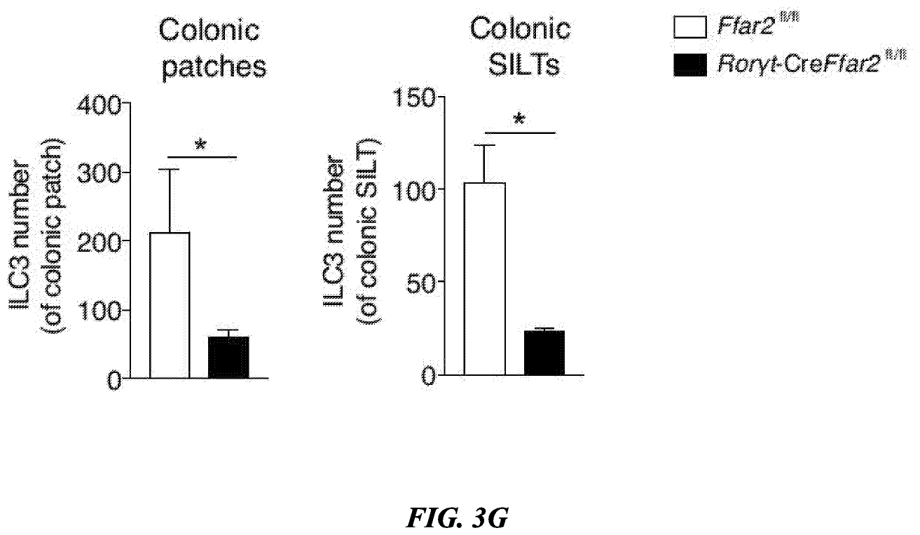

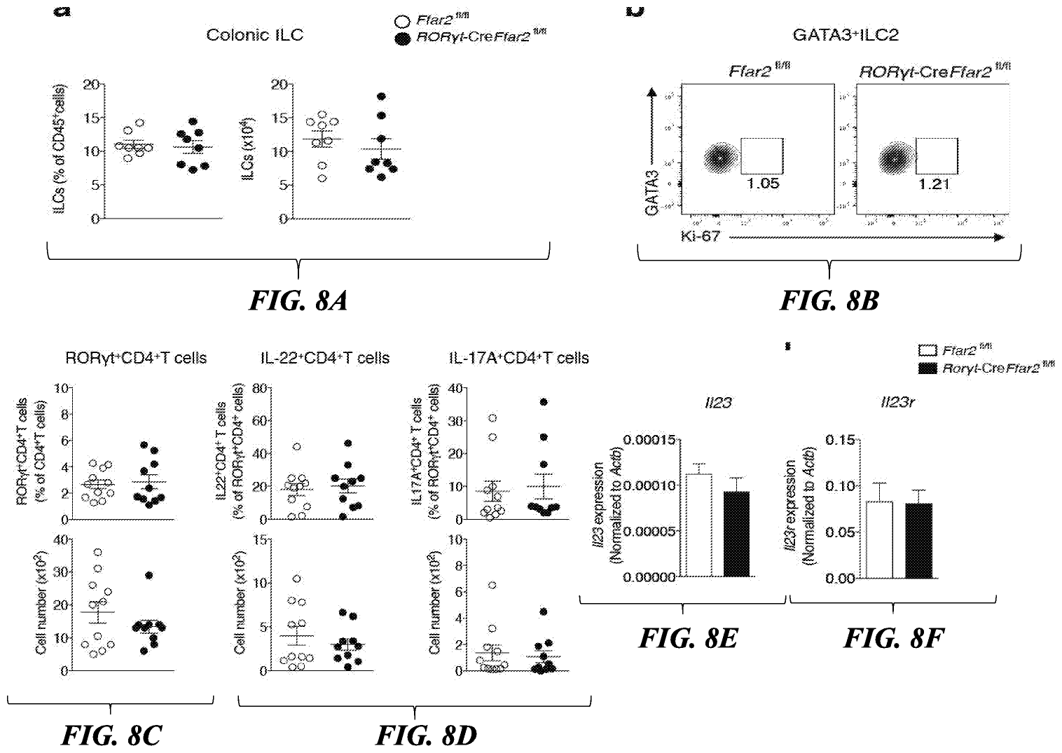

To decipher the role of Ffar2 in colonic ILC3s, RORγt-Cre Ffar2^{fl/fl} mice were generated. The population frequency and number of RORγt⁺ILC3 cells in RORγt-Cre Ffar2^{fl/fl} mice decreased compared to Ffar2^{fl/fl} mice, whereas total ILCs were unaffected by Ffar2 ablation (FIG. 2A and FIG. 8A). These data led us to ask whether Ffar2 regulates RORγt⁺ILC3 proliferation. Ki-67⁺ RORγt⁺ILC3 decreased in RORγt−Cre Ffar2^{fl/fl} mice compared to Ffar2^{fl/fl} mice, whereas Ki-67 expression in GATA3⁺ILC2s was not affected in either RORγt-Cre Ffar2^{fl/fl} or Ffar2^{fl/fl} mice (FIG. 2B and FIG. 8B). RORγt⁺ILC3s produce IL-22 and/or IL-17, both of which are key cytokines for ILC3 function^{1, 2}. Next, it was examined whether Ffar2 controls IL-22 and/or IL-17A production in RORγt⁺ILC3s. IL-22-producing ILC3s decreased whereas IL-17A-producing ILC3s were not altered in RORγt−Cre Ffar2^{fl/fl} mice compared to control (FIG. 3C). A subset of CD4⁺ T cells, T helper (Th) 17 cells, express RORγt and also produce IL-22 and/or IL-17^{41, 42}. In contrast to RORγt⁺ILC3s, RORγt−Cre Ffar2^{fl/fl} mice did not demonstrate alterations in colonic RORγt⁺ CD4⁺ T cell frequency and number (FIG. 8C). Neither IL-22 nor IL-17A production was affected in RORγt⁺ CD4⁺ T cells from RORγt−Cre Ffar2^{fl/fl} mice (FIG. 8D).

RORγt is a master transcription factor that regulates ILC3 development and function^{43, 44} and the aryl hydrocarbon receptor (AHR), a ligand-activated transcription factor, is also necessary for ILC3 proliferation and IL-22 production^{20, 21, 22.} It was observed that RORγt levels (MFI) in RORγt⁺ILC3s were reduced in the conditional knock-outs compared to controls while AHR expression was not affected in RORγt⁺ILC3s and IL-22 producing ILC3s (FIG. 2D-2E). Collectively, these data support that Ffar2 regulates colonic ILC3 proliferation and IL-22 production in a RORγt⁺ILC3 cell-intrinsic manner.

### Ffar2 Influences Colonic CCR6⁺ ILC3 Expansion and Function

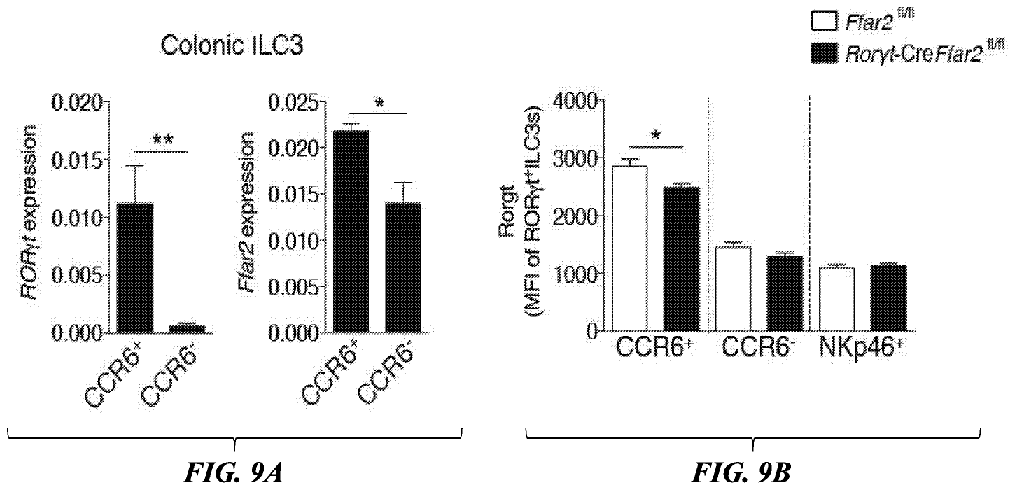

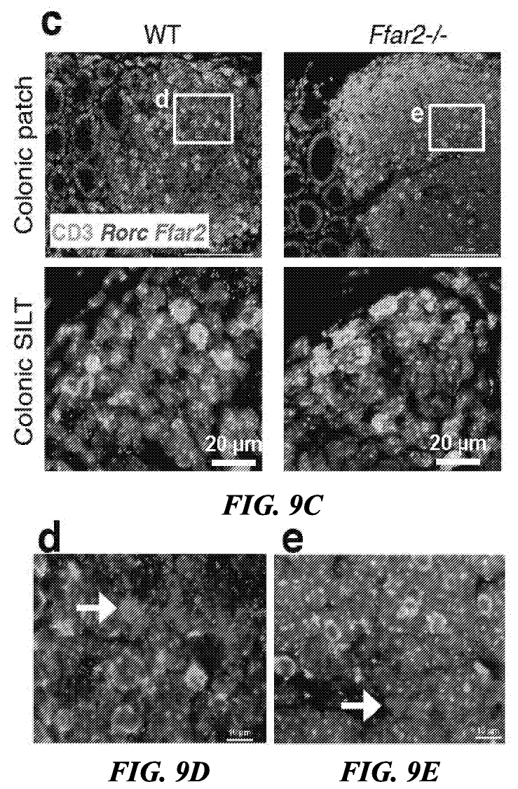

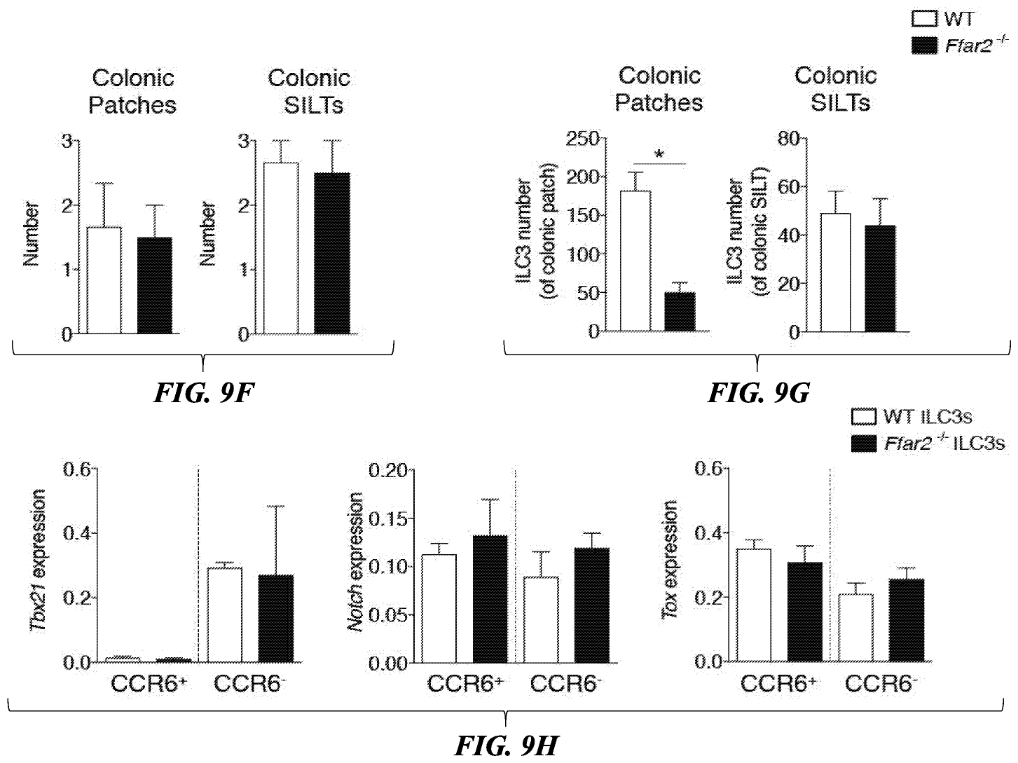

The observation that colonic CCR6⁺ ILC3s expressed higher levels of both Ffar2 and RORγt compared to CCR6⁻ ILC3s (FIG. 9A) led us to ask whether Ffar2 regulates CCR6⁺ ILC3 expansion and function. CCR6⁺ ILC3s, in contrast with CCR6⁻ ILC3s and NKp46⁺ ILC3s, were decreased in conditional knock-outs compared to controls (FIG. 3A). Consistent with analysis of mRNA expression, it was found that CCR6⁺ ILC3s expressed higher level of RORγt compared to CCR6⁻ ILC3s and NKp46⁺ ILC3s and that RORγt levels in CCR6⁺ ILC3s were reduced in the conditional knock-outs compared to controls (FIG. 9B). CCR6⁺ ILC3s exhibited higher expression of Ki-67 compared to CCR6⁻ and NKp46⁺ ILC3s in conditional knock-outs and controls (FIG. 3B). Both percentage and number of Ki-67⁺ CCR6⁺ ILC3s and Ki-67⁺ CCR6⁻ ILC3s decreased in RORγt−Cre Ffar2^{fl/fl} mice (FIG. 3B).

Both CCR6⁺ and CCR6⁻ ILC3s are known to produce IL-22¹⁰. It was observed that the majority of CCR6⁺ ILC3s were IL-22− producing cells and the number of IL-22⁺ CCR6⁺ ILC3s decreased in RORγt−Cre Ffar2^{fl/fl} mice (FIG. 3C). CCR6⁻ and NKp46⁺ ILC3s showed reduced levels of IL-22 production compared to CCR6⁺ ILC3s in both conditional knock-outs and controls (FIG. 3C), suggesting that Ffar2 expression may contribute to colonic CCR6⁺ ILC3s abundance and functional import through regulation of cell proliferation and IL-22 production.

Given the effects of Ffar2 expression on CCR6⁺ ILC3s, which are localized in colonic lymphoid tissues^{17, 45}, it was examined whether Ffar2-expression within ILC3s affected these tissue structures. To analyze the distribution of colonic Ffar2-expressing ILC3s, RNA in situ hybridization was employed, due to poor anti-Ffar2 antibody availability and quality for formalin-fixed paraffin embedded tissues, and visualized and quantified Ffar2-expressing ILC3s in WT and Ffar2^{-/-} mice. Colonic RORγt⁺ CD3⁻ ILC3s clustered in colonic patches, composed of B cells and T cells, and in colonic solitary intestinal lymphoid tissues (SILTs), composed of B cells, dendritic cells and a few T cells, in both WT and Ffar2^{-/-} mice (FIG. 9C-9E). The number of colonic patches and SILTs was not different between WT and Ffar2^{-/-} mice, while the number of RORγt⁺ CD3⁻ ILC3s in colonic lymphoid tissues decreased in Ffar2^{-/-} mice compared to WT mice (FIG. 9F-9G). Consistent with these data, the number of Ffar2⁺RORγt⁺ CD3⁻ ILC3s in colonic patches and SILTs was decreased in the conditional knock-out compared to controls; while the number of colonic patches and SILTs did not change in conditional knock-outs and controls (FIG. 3D-3G). Collectively, these data support that Ffar2 is not required for the development of colonic lymphoid tissues but rather may contribute to ILC3 expansion in and recruitment to colonic lymphoid tissues.

### Ffar2 Regulates ILC3-Derived IL-22 Via AKT and STAT3 Activation

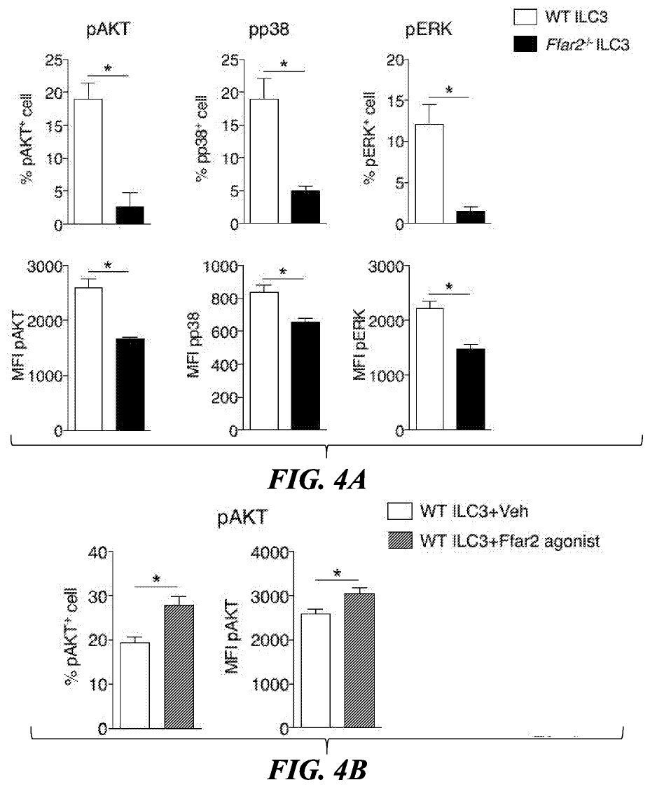

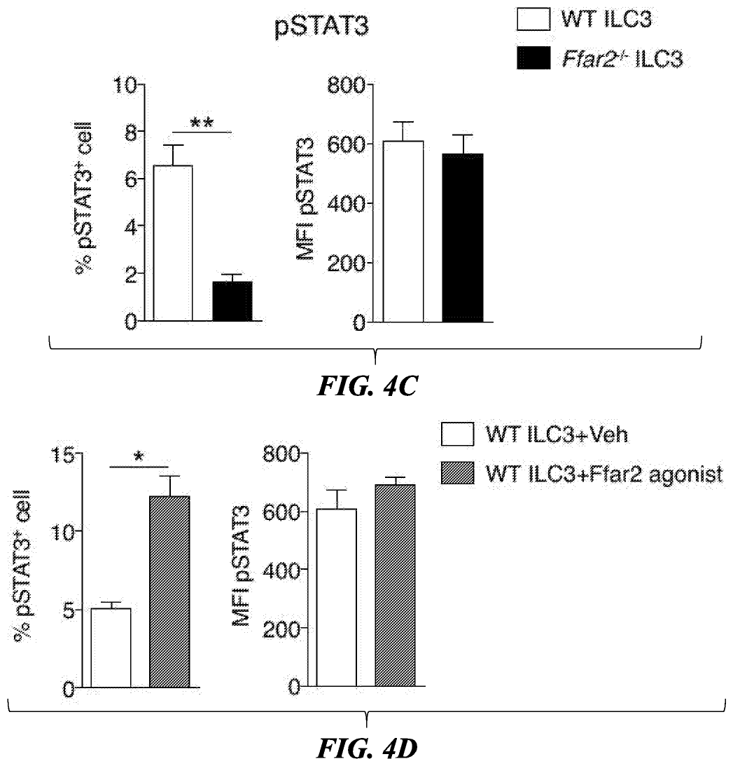

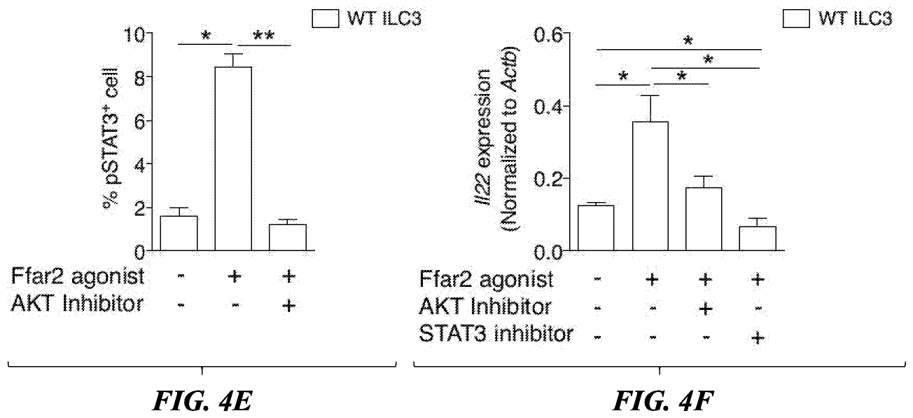

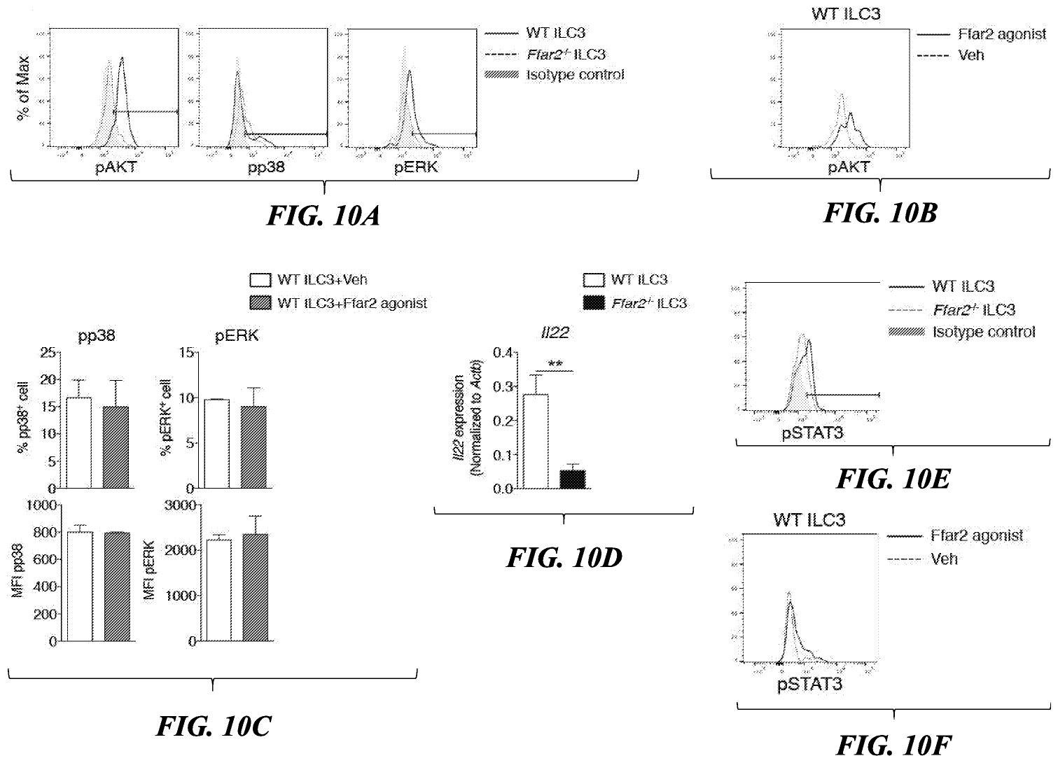

Ffar2 can couple with G_{i/o}, or G_{q} proteins and Ffar2 activation can inhibit cAMP and/or stimulate Ca²⁺ influx, eliciting intracellular signal cascades that regulate numerous cell-specific functions^{40, 46, 47}. Therefore, it was examined how Ffar2 signaling regulates colonic ILC3s and their function. Ffar2-deficient colonic ILC3s had reduced population frequencies and mean fluorescence intensities (MFIs) for phosphorylated AKT, p38, and ERK (FIG. 4A and FIG. 10A). Next, it was determined whether Ffar2 agonism activates these signaling molecules in colonic ILC3s. Ffar2 agonism increased phosphorylation of AKT, but not p38 and ERK in Ffar2-sufficient ILC3s (FIG. 4B and FIG. 10B-10C).

Phosphorylation of STAT3 (pSTAT3) is a significant regulator for ILC3-derived IL-22⁴⁸. Since Ffar2-deficient colonic ILC3s downregulated Il-22 expression (FIG. 10D) and RORγt−Cre Ffar2^{fl/fl} mice exhibited a reduction in IL-22− producing ILC3s (FIG. 2C), it was examined whether Ffar2 signaling regulates STAT3 activation in colonic ILC3s. Ffar2-deficient ILC3s showed a reduced level of pSTAT3⁺ compared to Ffar2-sufficient ILC3s, and Ffar2 agonism increased the frequency of pSTAT3 in Ffar2-sufficient ILC3s (FIGS. 4C-4D and FIGS. 10E-10F) suggesting Ffar2 signals can directly regulate STAT3 phosphorylation in ILC3, even though a smaller proportion of ILC3s respond to Ffar2 agonism as compared to the pAKT⁺ ILC3 population. These data prompted us to ask whether AKT activation downstream of Ffar2 directly affects the phosphorylation of STAT3 in ILC3s. It was discovered that inhibition of AKT via the AKT inhibitor (VIII), upon Ffar2 activation, impaired STAT3 activation in Ffar2-sufficient ILC3s (FIG. 4E). Next, signals downstream of Ffar2 activation were examined for their affect on ILC3-derived Il-22 production. Inhibition of STAT3 via a STAT3 inhibitor (S31), upon Ffar2 activation impaired Il-22 expression in colonic ILC3s. The AKT inhibitor also decreased 11-22, but to a lesser extent (FIG. 4F), suggesting Ffar2 agonism may be sufficient but not necessary for ILC3-derived IL-22. Together, these data demonstrate that Ffar2 signaling regulates colonic ILC3-derived Il-22 expression through an AKT and STAT3 axis.

### Ffar2-Expressing Colonic ILC3s Protect Against Intestinal Inflammation

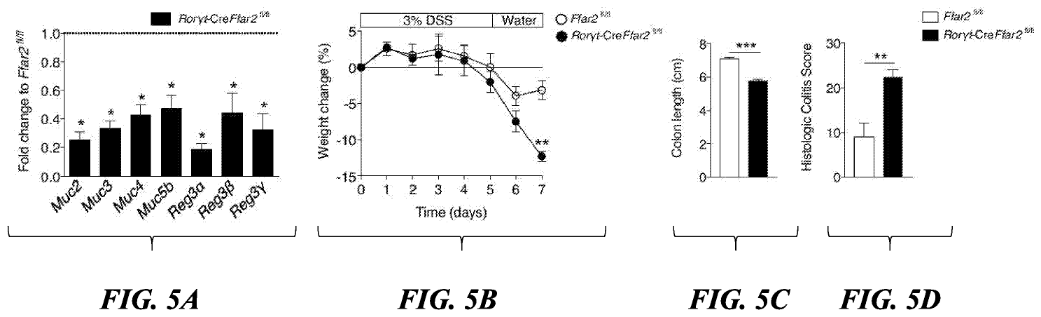

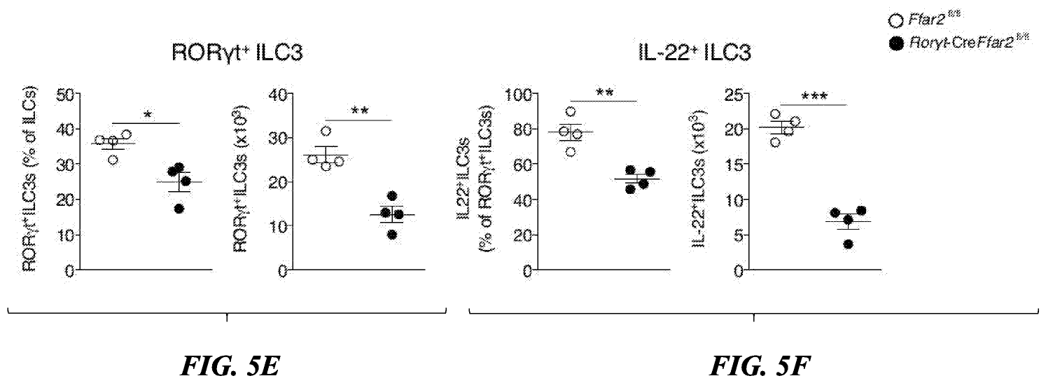

IL-22− producing ILC3s play significant roles not only in tissue homeostasis but also in host defense by regulating gut epithelial barrier function^{13, 14, 49, 50}. Analysis of targeted gut epithelial transcriptional signatures from RORγt−Cre Ffar2^{fl/fl} mice revealed decreased mucin (Muc2, Muc3, Muc4, and Muc5b) and antimicrobial peptide (Reg3α, Reg3β, and Reg3γ) expression (FIG. 5A). IL-22 can ameliorate dextran sulfate sodium (DSS)-induced colonic injury by enhancing mucus production⁵¹. To determine whether Ffar2-expressing RORγt⁺ ILC3s regulate intestinal injury repair responses, a DSS colonic injury model was employed. RORγt−Cre Ffar2^{fl/fl} mice treated with DSS showed increased weight loss, shortened colon length, worse histology-based colitis score, and decreased RORγt⁺ ILC3s and IL-22− producing ILC3s compare to Ffar2^{fl/fl} mice (FIG. 5B-5F).

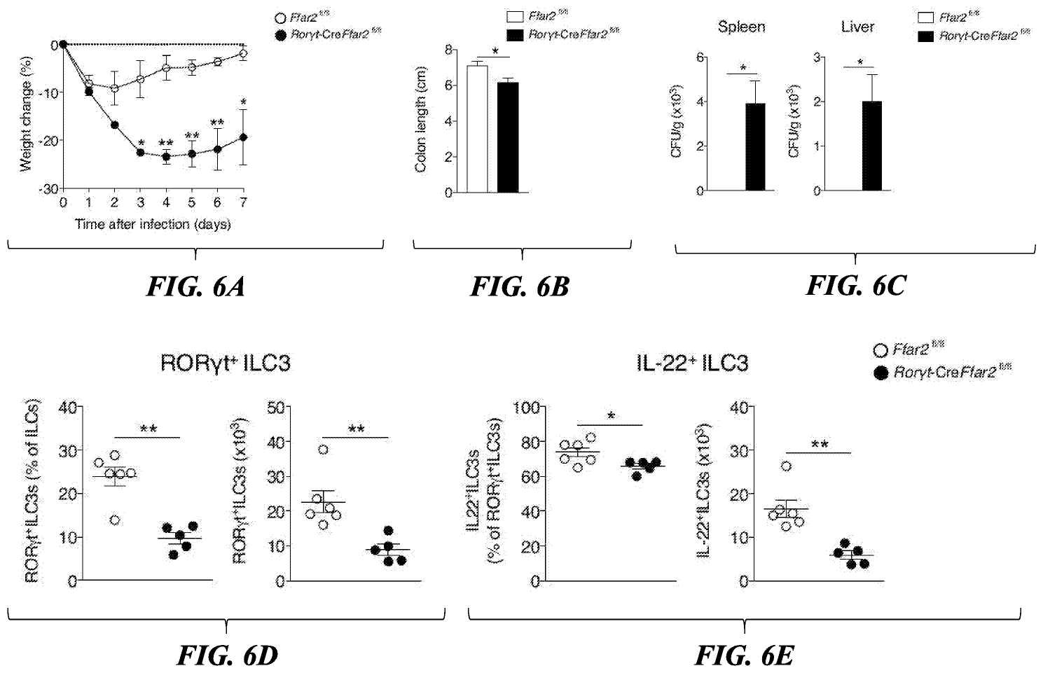

Antimicrobial peptides are a significant mechanism downstream of IL-22 that affords protection against attaching and effacing bacteria pathogens¹⁴. To test the role of Ffar2-expressing IL-22⁺ILC3s in bacterial infection, both RORγt−Cre Ffar2^{fl/fl} and Ffar2^{fl/fl} mice were inoculated with Citrobacter rodentium (C. rodentium). Consistent with the results in the DSS model; RORγt−Cre Ffar2^{fl/fl} mice displayed increased weight loss and shortened colon length, manifested higher levels of C. rodentium bacterial translocation to the liver and spleen, and reduced ILC3s and IL-22− producing ILC3s compared to Ffar2^{fl/fl} mice (FIGS. 6A-6E). Taken together, these data support that Ffar2 expression in ILC3 affords protection against large intestinal injury and bacterial infection.

### Discussion

Dietary and bacterial metabolites regulate immune responses and influence gut health and disease susceptibility. Some of these metabolites directly activate metabolite-sensing G-protein-coupled receptors (GPCRs) and these engagements induce immediate biological and immunological responses that contribute to mucosal homeostasis and intestinal immunity.

Herein, it was demonstrated that the metabolite-sensing GPCR Ffar2 is functionally expressed in colonic ILC3s and that Ffar2 agonism selectively promoted colonic ILC3 expansion and function. Ffar2 deficiency in ILC3s decreased their proliferation and IL-22 production and reduced expansion of ILC3s in colonic lymphoid tissues. Ffar2-deficient ILC3s downregulated AKT and MAPK signaling pathways. Ffar2 agonism specifically activated an ILC3 AKT and STA3 axis and regulated ILC3-derived IL-22 expression. In addition, Ffar2-deficient ILC3s led to impaired epithelial barrier function and resulted in increased susceptibility to intestinal injury and infection. The present study which unveils a heretofore unknown role for Ffar2 in ILC3 populations sheds light on a variety of aspects of ILC3 biology ranging from the contested role of the microbiota in affecting ILC3 populations to the transcriptional networks and their inputs that control ILC3.

Whether the microbiota affect ILC3 populations has remained controversial. Some studies suggest that germ-free mice exhibit a decrease in IL-22⁺NKp46⁺ ILC3s¹², whereas other studies have shown that the microbiota functions as a negative regulator of ILC3s in the small intestine⁴⁴. In these studies, colonic ILC3s decreased in germ-free WT mice and Ffar2 agonism by sodium acetate or an Ffar2 agonist increased colonic ILC3 numbers in germ-free WT mice (FIG. 7D-7F). Thus, these results provide additional evidence that the microbiota may regulate colonic ILC3 function and suggest that the microbial metabolite receptor Ffar2 serves as a positive regulator of colonic ILC3 expansion.

The present study also unveiled microbial metabolite receptor inputs for ILC3 transcriptional regulation. The master transcriptional factor, RORγt is significant for the regulation of ILC3 development and function. Besides RORγt, transcription factors such as Notch and AhR have been proposed to contribute to development of ILC3s⁵². While Notch signaling is especially important in fetal liver ILC3 precursors⁵³, AhR is necessary for adult ILC3 population maintenance and function. The AhR/ARNT complex promotes ILC3 survival by induction of Bcl-2, c-Kit, Il-7R, and Notch2 expression²¹. In addition, AhR together with RORγt regulate expression of IL-22 in the presence of IL-23²².

The previous studies support that Ffar2 is also a key regulator of ILC3 maintenance and function, and the data suggest that Ffar2 regulates RORγt levels in colonic ILC3s. It is not suggested that Ffar2 directly binds to RORγt or Il22 UTRs like Ahr complexes do; rather, signals downstream of Ffar2 mediate such effects. It was observed that Ffar2 signals activate AKT and MAKP kinase (p38 and ERK1/2) within ILC3s and generate adequate signal thresholds to maintain ILC3 pools within the total ILC population. Additionally, Ffar2 signaling led to STAT3 phosphorylation (pSTAT3) allowing optimized IL-22 production for gut homeostasis. Amplification of Ffar2 signals by Ffar2 agonism specifically induced AKT activation in ILC3s and increased pSTAT3⁺ ILC3s likely via AKT activation. The present data support that Ffar2 signaling actively participates in sensing environmental cues and establishing the tone and magnitude of ILC3 responses. In addition, by using RORγt−Cre Ffar2^{fl/fl} mice, wherein RORγt⁺ ILC3s are deficient in Ffar2, it was demonstrated a cell-intrinsic role for Ffar2 in colonic ILC3 proliferation and IL-22 production. In general, ILC3s are regulated by cell-extrinsic factors including cytokines, growth factors and dietary metabolites. IL-23 produced from CX3CR1⁺ macrophages and to some extent by CD103⁺CD11b⁺ dendritic cells is a key regulator of ILC3 activation^{54, 55}. RORγt−Cre Ffar2^{fl/fl} mice had similar expression levels of Il23 in the colon compared to Ffar2^{fl/fl} mice (FIG. 8E) and sorted Ffar2-sufficient and Ffar2-deficient colonic ILC3s showed the similar level of Il23R expression (FIG. 8F) suggesting the effect through IL-23 and IL-23R can be dampened in RORγt−Cre Ffar2^{fl/fl} mice. Thus, regulation of thresholds of Ffar2 signals by agonism or antagonism can be useful for ILC3 maintenance and lead to efficient gut homeostasis and defense.

ILC3 subsets share common functions and have transcriptionally and functionally distinct features in different tissue environments. Distribution of ILC3 subsets differs dependent upon tissue location. CCR6⁺ ILC3s (LTi-like ILC3s) are dominant in the colon and lymphoid tissues, whereas NKp46⁺ ILC3s (CCR6⁻ ILC3s) are prevalent in the small intestine and laminar propria¹⁹. It was observed that CCR6⁺ ILC3s were the majority of the colonic ILC3s and these cells were more proliferative and activated (higher IL-22 production) compared to CCR6⁻ ILC3 and NKp46⁺ ILC3s regardless of Ffar2 expression. This is in contrast with the small intestine, wherein CCR6⁺ ILC3s showed lower Ki-67 expression compared to NKp46⁺ ILC3s⁵⁶ and CCR6⁻ ILC3s produced higher amounts of cytokines including IL-22^{12, 44}. Since Ffar2 expression in CCR6⁺ ILC3s is most likely involved in their proliferation, IL-22 production and expansion; Ffar2 may affect CCR6⁺ ILC3s progenitor cells. CCR6⁺ ILC3s differentiate from lymphoid tissue inducer progenitors (LTiPs), while ILC1, ILC2, or ILC3 including CCR6⁻ and NKp46⁺ cells develop from ILC progenitors (ILCPs)³. Given that all colonic ILC subsets do not change in WT versus Ffar2^{-/-} mice or conditional knock-outs versus control mice, there seems to be no effect of Ffar2 on ILC3 development, but further analysis of each progenitor would be needed to confirm the role of Ffar2 in the development of CCR6⁺ ILC3s. Ffar2 can regulate transcriptional factors such as Notch in conjunction with T-bet and TOX, which have been proposed to contribute to development of NKp46⁺ ILC3s and LTi-like cells, respectively^{57, 58}. However, differences in Notch, T-bet, or TOX were not detected in sorted colonic CCR6⁺ or CCR6⁻ ILC3s from WT and Ffar2^{-/-} mice (FIG. 9H). The data support that Ffar2 regulates apoptotic/survival factors for colonic CCR6⁺ ILC3s. CCR6⁺ ILC3s in the small intestine expressed higher levels of the anti-apoptotic factor Bcl-2 compared to NKp46⁺ and CCR6⁻ ILC3s, and in general CCR6⁺ ILC3s are more resistant to γ_{c} cytokine depletion (IL-2, IL-7 or IL-15)⁵⁶. Further investigation of whether Ffar2 affects the expression of anti-apoptotic factors or IL-7R in colonic CCR6⁺ ILC3 may provide insight for a better understanding of the role of Ffar2 regulation in colonic ILC3 subsets. Notably, it was observed that Ffar2 induced accumulation of colonic ILC3s in lymphoid structures while Ffar2 is not likely required for lymphoid tissue formation. Whether Ffar2 is required for ILC3 recruitment to colonic lymphoid structures or whether Ffar2 mainly accelerates CCR6⁺ ILC3 expansion after the localization to colonic lymphoid tissues needs to be investigated further.

In summary, the present findings expand understanding of microbial metabolite-sensing receptors as significant regulators in ILC3 biology and ILC3-mediated mucosal immunity and elucidate the relevance of signal thresholds in regulating ILC3 function and ILC3 subset heterogeneity. To not be bound by a particular theory, this study raises the question of whether manipulating Ffar2 signaling thresholds modulate the plasticity of ILC3s, discriminates redundant or non-redundant ILC3 subset function in different tissue microenvironments, and regulates the onset or progression of intestinal injury and inflammation through fine-tuning of ILC3 responses. Ffar2 signaling appears to play a pivotal role in ILC3s, differentially coordinating interactions with adaptive immune cells or gut epithelial components and shaping ILC3-mediated gut immunity. Elucidation of the molecular links between the microbial metabolite-sensing receptor Ffar2 and ILC3 responses may lead to reconsideration and repositioning of Ffar2 as therapeutic target for treatment of intestinal diseases including inflammatory bowel disease.

### Example 2: Experimental Methods

Mice. C57BL/6J (wild-type) mice were bred in-house and originally purchased from Jackson Laboratory. Rorgt-Cre mice⁴³ on a C57BL/6J background were purchased from Jackson Laboratory and used only as heterozygotes. Ffar2^{fl/fl} mice on a C57BL/6J background were generously provided by Brian Layden (Northwestern University) and crossed with Rorgt-Cre mice to generate Rorgt-Cre x Ffar2^{fl/fl} mice. Ffar2^{-/-} mice were on C57BL/6 background and obtained from heterozygous x heterozygous breeding³⁵. Foxp3^{YFP-Cre} mice were bred in-house and originally provided by Dr. A. Rudensky (Memorial Sloan Kettering Cancer Center)⁵⁹. Littermate controls were used and animals were cohoused after weaning. Male and female mice were used at 7-12 weeks of age. In individual experiments, all animals were age and sex-matched; exact numbers of animal used per experiment are indicated in figure legends. All mice were housed in microisolator cages in the barrier facility of Harvard T.H. Chan School of Public Health. Animal studies and experiments were approved and carried out in accordance with Harvard Medical School's Standing Committee on Animals and the National Institutes of Health guidelines for animal use and care.

GPR43 agonist. Ffar2 agonist (ES43012-SOD) was synthesized and provided (See Bernard J. et al., 70th Scientific Sessions of the American Diabetes Association (Orlando, Fla.), Jun. 25-29th, 2010. Mice received 50 mg/kg/d of Ffar2 agonist dissolved in distilled water (5 ml/kg) provided by gentle oral administration twice a day for 1-2 weeks or the indicated duration. Germ-free (GF) mice were treated for 1-2 weeks with Ffar2 agonist (700 μM) dissolved in autoclaved drinking water and filtered sterilized.

SCFA intervention. WT mice were treated for 2 weeks with sodium acetate (150 mM) (S8750, Sigma), sodium propionate (150 mM) (P5436, Sigma), or sodium butyrate (100 mM) (303410, Sigma) in dissolved in their autoclaved drinking water and filter sterilized³⁵. WT mice received sodium chloride (150 mM). For GF mice, mice were treated for 2 weeks with sodium acetate (150 mM) and sodium chloride (150 mM) as a control in the drinking water. T-cell transfer colitis mice were treated for 6 weeks with sodium acetate (150 mM), sodium propionate (150 mM), sodium butyrate (100 mM), or sodium chloride (150 mM) as a control in the drinking water beginning at day 10. Drinking water solutions were freshly prepared and changed every 5 days.

Isolation of colonic epithelial cells and immune cells. Colons were dissected and fat and blood vessels were removed. Colons were cut open longitudinally and washed with PBS to remove feces and debris, then incubated in PBS containing 5 mM EDTA, 0.145 mg/ml Dithiothreitol (Sigma-Aldrich), 3% FBS and 1% penicillin/streptomycin (P/S) for 15 min at 37° C. for 2 cycles. After being vortexed for 15s, the dissociated cells were collected as colonic epithelial cells. For the isolation of lamina propria immune cells, the remaining colonic tissues were washed twice in PBS, cut into 1 mm in length, and digested in RPMI 1640 containing 0.5 mg/ml collagenase D (Roche), 0.01 mg/ml DNase I (Roche), and 0.5 mg/ml Dispase (Stem Cell Technologies) for 30 min at 37° C. on a shaking platform. The digested tissues were passed through 70 μm strainers after being vigorously vortexed for 15s. Then, colonic immune cells were collected and resuspended in PBS with 1% fetal bovine serum and 1% penicillin-streptomycin solution (Corning) for flow cytometry analysis, FACSAria sorting or RNA extraction.

Flow cytometry. Single-cell suspensions were stained with a combination of fluorescently conjugated monoclonal antibodies. CD16/32 antibody (93; BioLegend) was used to block the non-specific binding to Fc receptors before surface staining. Cells were stained with Fixable yellow dead cell stain kit (Invitrogen) for the detection of live/dead cells before staining of the cell surface. All antibodies were purchased from BioLegend unless otherwise specified. For surface marker staining, antibodies to the following mouse proteins were used: CD45 (30-F11), CD90.2 (53-2.1), lineage markers (17A2/RB6-8C5/RA3-6B2/Ter-119/M1/70), CCR6 (29-21.17), NKp46 (29A1.4), CD11b (M1/70), CD11c (N418), Gr-1 (RB6-8C5), MHC class II (M5/114.15.2), NK1.1 (PK136), KLRG1 (2F1), CD3c (145-2C11), CD4 (RM4.5).

For measurement of intracellular cytokine expression, cells were isolated ex vivo and stimulated with 50 ng/ml phorbol-12-myristate 13-acetate (PMA, Sigma-Aldrich) and 500 ng/ml ionomycin (Sigma-Aldrich) and Brefeldin (1000× solution, BioLegend) for 4 hr. Cells were subsequently surface-stained with a combination of the antibodies listed above, fixed and permeabilized using Foxp3 Fix/Perm Buffer set (BioLegend), and stained with IL-22− PerCP eFluor710 (1H8PWSR; eBioscience), and IL-17A-Alexa Fluor 488 (eBiol7B7; eBioscience).

For transcription factor expression, cell were isolated directly ex vivo, stained with antibodies to surface antigens, fixed and permeabilized according to the manufacturer's instructions (Foxp3 Fix/Perm Buffer set, BioLegend) and stained with phycoerythrin (PE) or Allophycocyanin (APC)-conjugated RORgt (B2D, Invitrogen), Alexa Fluor 488-conjugated GATA3 (L50-823, BD Biosciences), eFluor 660-conjugated Ahr (4MEJJ, eBioscience), Alexa 647-conjugated T-bet (4B10), PE-conjugated Foxp3 (FJK-16s, eBioscience), and PerCP eFluor710-conjugated Ki-67 (SolA15, eBiosciences).

For analysis of intracellular signaling in ILC3s (Ibiza et al., 2016), sorted colonic ILC3s were rested for 2 hours in RPMI at 37° C. Cells were unstimulated or stimulated with Ffar2 agonist (10 μM/ml dissolved in water, pH 7.4) for 30 min at 37° C., fixed and permeabilized according to the manufacturer's instructions (BD Cytofix and BD Phosflow Perm Buffer III, BD Biosciences), and stained with Alexa Fluor 647-conjugated anti-AKT (pS473) (D9E, Cell Signaling Technology), anti-p38 MAPK (pT180/pY182) (36/038, BD Biosciences), anti-pERK1/2(pT202/pY204) (E10, Cell Signaling Technology) or anti-STAT3 (pY705) (4/p-STAT3, BD Biosciences) for 30 min at room temperature.

Cells were stained in parallel with the respective control isotype antibodies. FMO controls were performed as well. Stained cells were acquired on a BD LSRII flow cytometry (BD Biosciences) and analyzed with FlowJo9 software (Tree Star). Colonic lamina propria immune cells were sorted to >95% purity using a FACSAria Hu at the Dana-Farber Cancer Institute Flow Cytometry Core.

RNA isolation and real-time quantitative PCR (qRT-PCR). For analysis of sorted colonic immune cell populations including ILC subsets and colonic ILC3s, RNA was isolated using the RNeasy Micro Kit (Qiagen) or RNeasy Mini Kit (Qiagen) according to the manufacturer's protocol. For analysis of colonic epithelial cells or colon tissues, cells or tissues were homogenized directly into Qiazol (Qiagen), and RNA was isolated via chloroform extraction. The quantity and quality of RNA was determined using a NanoDrop (Thermo Scientific). For both methods, cDNA was synthesized with iScript Reverse Transcription Supermix for RT-qPCR (Bio-Rad). Quantitative real-time PCR was carried out on cDNA with SYBR FAST Universal qPCR Master Mix (KAPA Biosystems). Reactions were run on a Stratagene Mx3005P machine (Agilent Technologies). The expression of individual genes was normalized to housekeeping gene β-actin expression on the base of the ΔΔCt algorithm. Some results are shown as a fold induction relative to expression levels in colonic epithelial cells as indicated. Primer sequences are listed in TABLE 2 below.

TABLE 2
REAL-TIME PCR PRIMER SEQUENCES

| Gene | Forward (5′-3′) | Reverse (5′-3′) |
|---|---|---|
| Ffar2 | AATTTCCTGGTGTGCTTTGG | ACCAGACCAACTTCTGGGTG |
| Rorc | GACCCACACCTCACAAATTGA | AGTAGGCCACATTACACTGCT |
| Il22 | ATGAGTTTTTCCCTTATGGGG | GCTGGAAGTTGGACACCTCAA |
| | AC | |
| Il22rα1 | ATGAAGACACTACTGACCATC | CAGCCACTTTCTCTCTCCGT |
| | CT | |
| Il23r | TTCAGATGGGCATGAATGTTT | CCAAATCCGAGCTGTTGTTCT |
| | CT | AT |
| Ccl20 | GTACTGCTGGCTCACCTCTG | TCCAATTCCATCCCAAAA |
| Muc2 | CAATGACAAGGTGTCCTGCC | GTGCTCTCCAAACTCTCTGG |
| Muc3 | CCGATGTCACCACTTCTGCTG | GCAGAGCAAGGCGTGATACAG |
| Muc4 | TGATGGAACAACCACCTCAC | GGATGCAGGTGAGGTATTC |
| Muc5b | GTGGCCTTGCTCATGGTGT | GGACGAAGGTGACATGCCT |
| Reg3α | TCACCTGGTCCTCAACAGTAT | GGAGCGATAAGCCTTGTAACC |
| | T | |
| Reg3β | CAGACCTGGTTTGATGCAGA | GAAGCCTCAGCGCTATTGAG |
| Reg3γ | TTCCTGTCCTCCATGATCAAA | CATCCACCTCTGTTGGGTTCA |
| | A | |
| Actin | TACCACCATGTACCCAGGCA | CTCAGGAGGAGCAATGATCTT |
| | | GA |

For quantitative RT-PCR of Tbx21, Notch, and Tox, RNA was retro-transcribed using a High Capacity RNA-to-cDNA Kit (Applied Biosystems), followed by a pre-amplification PCR using TaqMan PreAmp Master Mix (Applied Biosystems). TaqMan Gene Expression Master Mix (Applied Biosystems) was used in real-time PCR. TaqMan Gene Expression Assays bought from Applied Biosystems were the following: Gapdh Mm99999915_g1; Hprt1 Mm00446968_m1; Eefla1 Mm01973893_g1; Tbx21 Mm00450960_m1; Notch2 Mm00803077_m1; Tox Mm00455231_m1. Hprt1, Gapdh and Edfala1 were used as housekeeping genes.

Histology. Colons were cleaned with PBS prior to fixation in 4% PFA and then processed by routine paraffin embedding, sectioning and H&E staining. Colitis scores were determined by a pathologist (J. N. G.), who was blinded to the experimental parameters. Each of the four histologic parameters was scored as absent (0), mild (1), moderate (2), or severe (3): mononuclear cell infiltration, polymorphonuclear cell infiltration, epithelial hyperplasia, and epithelial injury. The scores for the parameters were summed to generate the cumulative histologic colitis score as previously described^{60, 61}. For the DSS-induced model, cumulative histologic scores were also quantified as to the percentage involvement by the disease process: (1)<10%; (2) 10-25%; (3) 30-50%; (4) >50% and presented as histologic colitis scores⁶² as follows: (cumulative score*involvement)+cumulative score.

RNA in situ hybridization and immunofluorescence staining. To detect Ffar2-expressing RORγt⁺ CD3⁻ ILC3s in the colon, RNA in situ hybridization was performed, then subsequently carried out immunofluorescence staining. Mouse colon tissues were fixed overnight in 10% neutral buffered formalin (NBF) followed by routine paraffin embedding and sectioning. RNA in situ hybridization was performed using the Advanced Cell Diagnostic RNAscope Multiplex Fluorescent Detection Kit v2 (323100, ACDBio) according to the manufacturer's instructions⁶³. In brief, colon sections were deparaffinized, pretreated with Target Retrieval Reagents and protease, hybridized with Mm-Ffar2 probe (433711, ACDBio) and Mm-Rorc-C2 probe (403661-C2, ACDBio), and then underwent amplification steps. Two different chromogenic substrates: TSA Cyanine 5 (NEL745E001KT, PerkinElmer) and TSA cyanine 3 (NEL744E001KT, PerkinElmer) were used to detect the Rorc and Ffar2 probes, respectively. Prior to DAPI counterstaining, immunofluorescence staining was performed following the Advanced Cell Diagnostic general recommendations (323100-TN). Briefly, the sample slides were blocked in Tris-buffered saline (TBS) 1% BSA and 5% goat serum for 1h at room temperature, stained with anti-CD3 primary antibody (Rabbit polyclonal, 5690, Abcam) overnight at 4° C. and then donkey anti-rabbit-HRP secondary antibody (711-035-152, Jackson ImmunoResearch) for 2h at room temperature. To detect CD3 positive cells, TSA Fluorescein reagent (NEL741E001KT, PerkinElmer) was used. The slides were counterstained with DAPI and mounted with Prolong Gold antifade mounting medium (P36934, Life Technologies). Images were acquired on a Nikon Eclipse NI-U equipped with a 20×, a 40× or a 60× objective or Nikon Eclipse Ti laser scanning microscope coupled with a 100× objective. Image analysis was performed using ImageJ. The number of colonic patches and SILTs were counted in whole colon tissue sections. For quantification of colonic ILC3s in the colonic tissues, 10 digital images of colonic patches or colonic SILTs were selected. The number of Rorc⁺ CD3⁻ ILC3 number per each lymphoid tissue was counted by subtracting the number of Rorc⁺ CD3⁺ double positive cells from that of Rorc⁺ cells with DAPI-positive signals. M.M. or E.C. performed quantification and were blinded to sample experimental identity.

AKT and STAT3 inhibition. Sorted colonic ILC3s were rested for 2 hours in RPMI at 37° C. For analysis of STAT3 phosphorylation, cells were incubated with 1004 AKT inhibitor VIII (VIII, Sigma-Aldrich) (Ibiza et al., 2016) or vehicle as a control (DMSO) for 1h at 37° C. before stimulation with the Ffar2 agonist (10 μM/ml dissolved in water, pH 7.4). To determine Il22 expression levels, cells were incubated with 10 μM AKT inhibitor VIII, 10 μM STAT3 inhibitor VI (S3I, Sigma-Aldrich) (Ibiza et al., 2016) or vehicle during overnight stimulation with Ffar2 agonist at 37° C.

T cell-transfer colitis model. Splenic T cells (CD3⁺CD4⁺CD25⁻CD45RB^{hi}) were sorted from C57BL/6J WT mice and injected i.p. into C57BL/6J Rag2^{-/-} mice (5×10⁵ cells/mouse). At day 10 post-splenic T cell injection, splenic Treg cells (CD3⁺CD4⁺Foxp3^{YFP+}) were sorted from Foxp3^{YFP-Cre} mice and adoptively transferred to recipients by i.v. injection (1×10⁵ cells/mouse). Mice were monitored weekly for weight loss and morbidity for 6-8 weeks as per the protocol's experimental endpoint guidelines and sacrificed at the terminal time point of 6 weeks post injection of splenic Treg cells.

DSS-induced colonic injury. Mice were treated with 3% (w/v) DSS (MP Biomedicals) ad libitum in drinking water for 5 days and followed by normal drinking water for 2 days. Body weight was measured every day and mice were euthanized at day 7. Colon length was measured. Colon was fixed with 4% paraformaldehyde for histology or used for the isolation of colonic immune cells or colonic epithelial cells as describe above.

Citrobacter rodentium infection. Citrobacter rodentium (DBS100 strain) was generously provided. Rorgt-Cre Ffar2^{fl/fl} mice or Ffar2^{fl/fl} mice were orally infected with 4×10⁹ CFU of C. rodentium⁶⁴. Mice were weighed daily. On day 7 after infection, the colon, spleen and liver were collected from the infected mice. For assessment of bacterial translocation⁶⁵, spleen and liver were weighed and homogenized with a TissueRuptor (Qiagen). The homogenates were plated on MacConkey agar plate and counted after overnight incubation at 37° C. under aerobic conditions. Colon length was measured and colons were used for the isolation of colonic immune cells or colonic epithelial cells as describe above.

Statistics. Data were analyzed with GraphPad Prism (version 7.0b). All data are represented as mean±s.e.m. For comparison between two independent experimental groups, an unpaired two-tailed Student's t-test when data were normally distributed or a two-tailed Mann-Whitney test was used. For comparison between more than two groups, one-way ANOVA followed by Tukey's test or by Dunnett's test was performed. No samples were excluded from any experiments performed in this study. Mice were randomized to experimental groups on weaning or 1 week prior to the start of an experimental intervention to avoid caged-based or housing bias. No blinding was used except for assignment of histologic scores and microscopy-based counting as noted above. Differences of P<0.05 were considered statistically significant.

### REFERENCES—EXAMPLE 1 AND EXAMPLE 2

1. Spits, H. & Cupedo, T. Innate lymphoid cells: emerging insights in development, lineage relationships, and function. Annu Rev Immunol 30, 647-675 (2012).
2. Artis, D. & Spits, H. The biology of innate lymphoid cells. Nature 517, 293-301 (2015).
3. Vivier, E. et al. Innate Lymphoid Cells: 10 Years On. Cell 174, 1054-1066 (2018).
4. Diefenbach, A., Colonna, M. & Koyasu, S. Development, differentiation, and diversity of innate lymphoid cells. Immunity 41, 354-365 (2014).
5. Eberl, G., Colonna, M., Di Santo, J. P. & McKenzie, A. N. Innate lymphoid cells. Innate lymphoid cells: a new paradigm in immunology. Science 348, aaa6566 (2015).
6. McKenzie, A. N. J., Spits, H. & Eberl, G. Innate lymphoid cells in inflammation and immunity. Immunity 41, 366-374 (2014).
7. Sonnenberg, G. F. & Artis, D. Innate lymphoid cells in the initiation, regulation and resolution of inflammation. Nat Med 21, 698-708 (2015).
8. Eberl, G. et al. An essential function for the nuclear receptor RORgamma(t) in the generation of fetal lymphoid tissue inducer cells. Nat Immunol 5, 64-73 (2004).
9. Klose, C. S. et al. A T-bet gradient controls the fate and function of CCR6-RORgammat+ innate lymphoid cells. Nature 494, 261-265 (2013).
10. Klose, C. S. & Artis, D. Innate lymphoid cells as regulators of immunity, inflammation and tissue homeostasis. Nat Immunol 17, 765-774 (2016).
11. Sawa, S. et al. Lineage relationship analysis of RORgammat+ innate lymphoid cells. Science 330, 665-669 (2010).
12. Satoh-Takayama, N. et al. Microbial flora drives interleukin 22 production in intestinal NKp46+ cells that provide innate mucosal immune defense. Immunity 29, 958-970 (2008).
13. Sonnenberg, G. F., Monticelli, L. A., Elloso, M. M., Fouser, L. A. & Artis, D. CD4(+) lymphoid tissue-inducer cells promote innate immunity in the gut. Immunity 34, 122-134 (2011).
14. Zheng, Y. et al. Interleukin-22 mediates early host defense against attaching and effacing bacterial pathogens. Nat Med 14, 282-289 (2008).
15. Gladiator, A., Wangler, N., Trautwein-Weidner, K. & LeibundGut-Landmann, S. Cutting edge: IL-17-secreting innate lymphoid cells are essential for host defense against fungal infection. J Immunol 190, 521-525 (2013).
16. Eberl, G. Inducible lymphoid tissues in the adult gut: recapitulation of a fetal developmental pathway? Nat Rev Immunol 5, 413-420 (2005).
17. Baptista, A. P. et al. Colonic patch and colonic SILT development are independent and differentially regulated events. Mucosal Immunol 6, 511-521 (2013).
18. Buettner, M. & Lochner, M. Development and Function of Secondary and Tertiary Lymphoid Organs in the Small Intestine and the Colon. Front Immunol 7, 342 (2016).
19. Withers, D. R. & Hepworth, M. R. Group 3 Innate Lymphoid Cells: Communications Hubs of the Intestinal Immune System. Front Immunol 8, 1298 (2017).
20. Kiss, E. A. et al. Natural aryl hydrocarbon receptor ligands control organogenesis of intestinal lymphoid follicles. Science 334, 1561-1565 (2011).
21. Lee, J. S. et al. AHR drives the development of gut ILC22 cells and postnatal lymphoid tissues via pathways dependent on and independent of Notch. Nat Immunol 13, 144-151 (2011).
22. Qiu, J. et al. The aryl hydrocarbon receptor regulates gut immunity through modulation of innate lymphoid cells. Immunity 36, 92-104 (2012).
23. Mielke, L. A. et al. Retinoic acid expression associates with enhanced IL-22 production by gammadelta T cells and innate lymphoid cells and attenuation of intestinal inflammation. J Exp Med 210, 1117-1124 (2013).
24. Ibiza, S. et al. Glial-cell-derived neuroregulators control type 3 innate lymphoid cells and gut defence. Nature 535, 440-443 (2016).
25. Saha, S., Rajpal, D. K. & Brown, J. R. Human microbial metabolites as a source of new drugs. Drug Discov Today 21, 692-698 (2016).
26. Rooks, M. G. & Garrett, W. S. Gut microbiota, metabolites and host immunity. Nat Rev Immunol 16, 341-352 (2016).
27. Tan, J. et al. The role of short-chain fatty acids in health and disease. Adv Immunol 121, 91-119 (2014).
28. Trompette, A. et al. Gut microbiota metabolism of dietary fiber influences allergic airway disease and hematopoiesis. Nat Med 20, 159-166 (2014).
29. Erny, D. et al. Host microbiota constantly control maturation and function of microglia in the CNS. Nat Neurosci 18, 965-977 (2015).
30. Perry, R. J. et al. Acetate mediates a microbiome-brain-beta-cell axis to promote metabolic syndrome. Nature 534, 213-217 (2016).
31. Thorburn, A. N., Macia, L. & Mackay, C. R. Diet, metabolites, and “western-lifestyle” inflammatory diseases. Immunity 40, 833-842 (2014).
32. Koh, A., De Vadder, F., Kovatcheva-Datchary, P. & Backhed, F. From Dietary Fiber to Host Physiology: Short-Chain Fatty Acids as Key Bacterial Metabolites. Cell 165, 1332-1345 (2016).
33. Tan, J. K., McKenzie, C., Marino, E., Macia, L. & Mackay, C. R. Metabolite-Sensing G Protein-Coupled Receptors-Facilitators of Diet-Related Immune Regulation. Annu Rev Immunol 35, 371-402 (2017).
34. Maslowski, K. M. et al. Regulation of inflammatory responses by gut microbiota and chemoattractant receptor GPR43. Nature 461, 1282-1286 (2009).
35. Smith, P. M. et al. The microbial metabolites, short-chain fatty acids, regulate colonic Treg cell homeostasis. Science 341, 569-573 (2013).
36. Marino, E. et al. Gut microbial metabolites limit the frequency of autoimmune T cells and protect against type 1 diabetes. Nat Immunol 18, 552-562 (2017).
37. Forbes, S. et al. Selective FFA2 Agonism Appears to Act via Intestinal PYY to Reduce Transit and Food Intake but Does Not Improve Glucose Tolerance in Mouse Models. Diabetes 64, 3763-3771 (2015).
38. Kim, S. H., Cho, B. H., Kiyono, H. & Jang, Y. S. Microbiota-derived butyrate suppresses group 3 innate lymphoid cells in terminal ileal Peyer's patches. Sci Rep 7, 3980 (2017).
39. Cummings, J. H., Pomare, E. W., Branch, W. J., Naylor, C. P. & Macfarlane, G. T. Short chain fatty acids in human large intestine, portal, hepatic and venous blood. Gut 28, 1221-1227 (1987).
40. Le Poul, E. et al. Functional characterization of human receptors for short chain fatty acids and their role in polymorphonuclear cell activation. J Biol Chem 278, 25481-25489 (2003).
41. Ivanov, I I et al. The orphan nuclear receptor RORgammat directs the differentiation program of proinflammatory IL-17+T helper cells. Cell 126, 1121-1133 (2006).
42. Korn, T., Bettelli, E., Oukka, M. & Kuchroo, V. K. IL-17 and Th17 Cells. Annu Rev Immunol 27, 485-517 (2009).
43. Eberl, G. & Littman, D. R. Thymic origin of intestinal alphabeta T cells revealed by fate mapping of RORgammat+ cells. Science 305, 248-251 (2004).
44. Sawa, S. et al. RORgammat+ innate lymphoid cells regulate intestinal homeostasis by integrating negative signals from the symbiotic microbiota. Nat Immunol 12, 320-326 (2011).
45. Emgard, J. et al. Oxysterol Sensing through the Receptor GPR183 Promotes the Lymphoid-Tissue-Inducing Function of Innate Lymphoid Cells and Colonic Inflammation. Immunity 48, 120-132 e128 (2018).
46. Brown, A. J. et al. The Orphan G protein-coupled receptors GPR41 and GPR43 are activated by propionate and other short chain carboxylic acids. J Biol Chem 278, 11312-11319 (2003).
47. Nilsson, N. E., Kotarsky, K., Owman, C. & Olde, B. Identification of a free fatty acid receptor, FFA2R, expressed on leukocytes and activated by short-chain fatty acids. Biochem Biophys Res Commun 303, 1047-1052 (2003).
48. Guo, X. et al. Induction of innate lymphoid cell-derived interleukin-22 by the transcription factor STAT3 mediates protection against intestinal infection. Immunity 40, 25-39 (2014).
49. Sanos, S. L., Vonarbourg, C., Mortha, A. & Diefenbach, A. Control of epithelial cell function by interleukin-22-producing RORgammat+ innate lymphoid cells. Immunology 132, 453-465 (2011).
50. Rutz, S., Eidenschenk, C. & Ouyang, W. IL-22, not simply a Th17 cytokine. Immunol Rev 252, 116-132 (2013).
51. Sugimoto, K. et al. IL-22 ameliorates intestinal inflammation in a mouse model of ulcerative colitis. J Clin Invest 118, 534-544 (2008).
52. Serafini, N., Vosshenrich, C. A. & Di Santo, J. P. Transcriptional regulation of innate lymphoid cell fate. Nat Rev Immunol 15, 415-428 (2015).
53. Cherrier, M., Sawa, S. & Eberl, G. Notch, Id2, and RORgammat sequentially orchestrate the fetal development of lymphoid tissue inducer cells. J Exp Med 209, 729-740 (2012).
54. Longman, R. S. et al. CX(3)CR1(+) mononuclear phagocytes support colitis-associated innate lymphoid cell production of IL-22. J Exp Med 211, 1571-1583 (2014).
55. Kinnebrew, M. A. et al. Interleukin 23 production by intestinal CD103(+)CD11b(+) dendritic cells in response to bacterial flagellin enhances mucosal innate immune defense. Immunity 36, 276-287 (2012).
56. Robinette, M. L. et al. IL-15 sustains IL-7R-independent ILC2 and ILC3 development. Nat Commun 8, 14601 (2017).
57. Rankin, L. C. et al. The transcription factor T-bet is essential for the development of NKp46+ innate lymphocytes via the Notch pathway. Nat Immunol 14, 389-395 (2013).
58. Aliahmad, P., de la Tone, B. & Kaye, J. Shared dependence on the DNA-binding factor TOX for the development of lymphoid tissue-inducer cell and NK cell lineages. Nat Immunol 11, 945-952 (2010).
59. Bjursell, M. et al. Improved glucose control and reduced body fat mass in free fatty acid receptor 2-deficient mice fed a high-fat diet. Am J Physiol Endocrinol Metab 300, E211-220 (2011).
60. Garrett, W. S. et al. Communicable ulcerative colitis induced by T-bet deficiency in the innate immune system. Cell 131, 33-45 (2007).
61. Garrett, W. S. et al. Colitis-associated colorectal cancer driven by T-bet deficiency in dendritic cells. Cancer Cell 16, 208-219 (2009).
62. Dieleman, L. A. et al. Chronic experimental colitis induced by dextran sulphate sodium (DSS) is characterized by Th1 and Th2 cytokines. Clin Exp Immunol 114, 385-391 (1998).
63. Wang, F. et al. RNAscope: a novel in situ RNA analysis platform for formalin-fixed, paraffin-embedded tissues. J Mol Diagn 14, 22-29 (2012).
64. Crepin, V. F., Collins, J. W., Habibzay, M. & Frankel, G. Citrobacter rodentium mouse model of bacterial infection. Nat Protoc 11, 1851-1876 (2016).
65. Bhinder, G. et al. The Citrobacter rodentium mouse model: studying pathogen and host contributions to infectious colitis. J Vis Exp, e50222 (2013).

### Example 3: FFar2 Regulates Colonic ILC3-Derived IL-22 Via AKT and STAT3 Activation

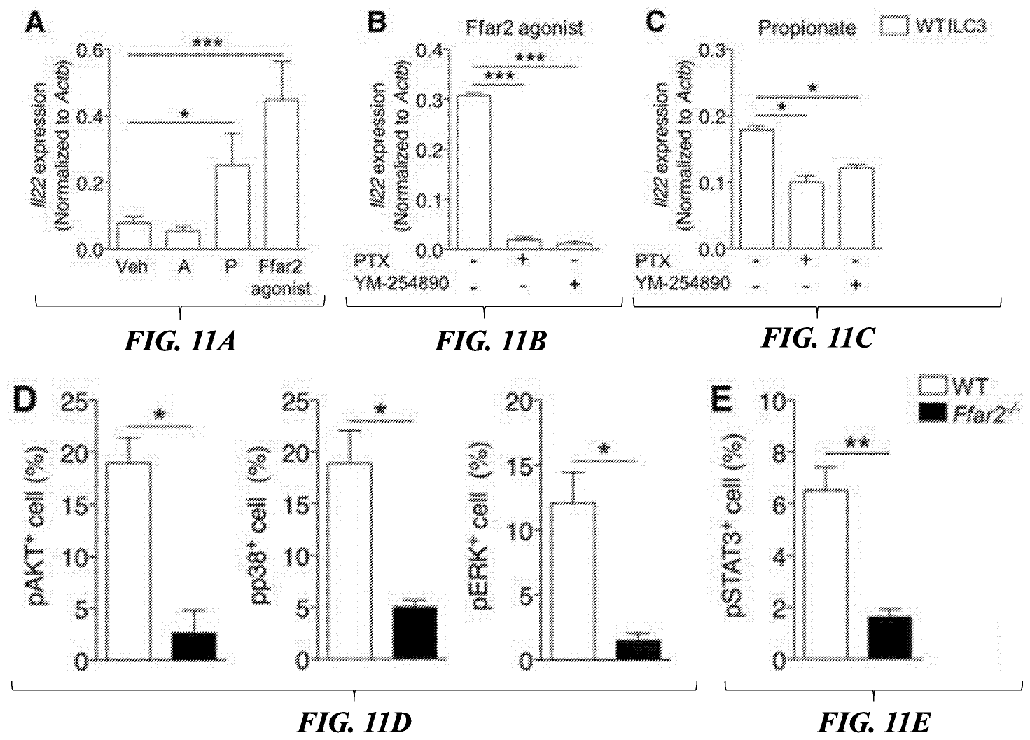

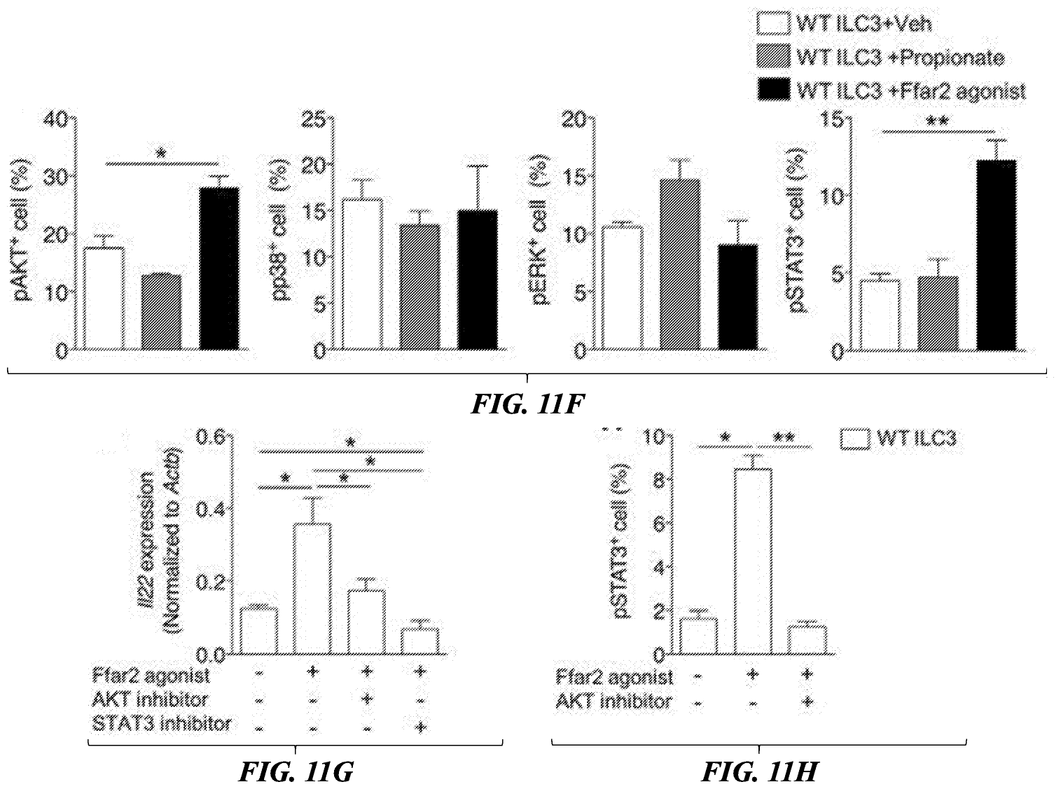

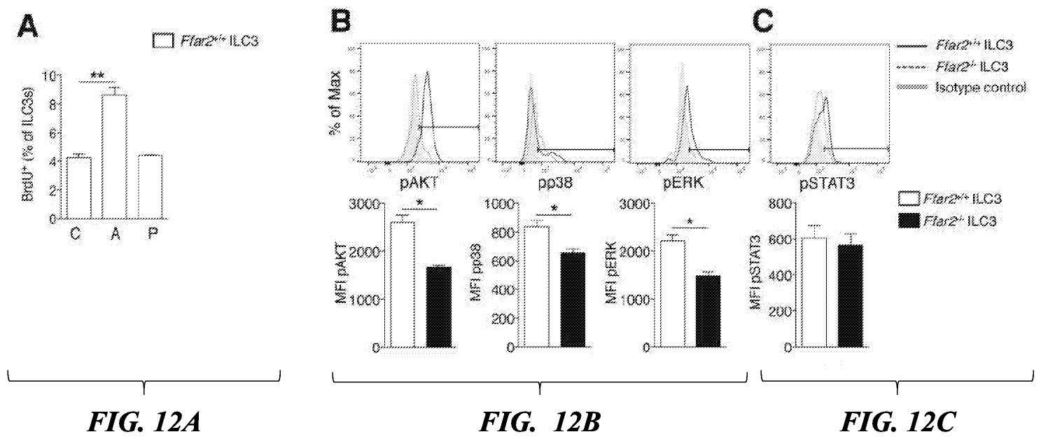

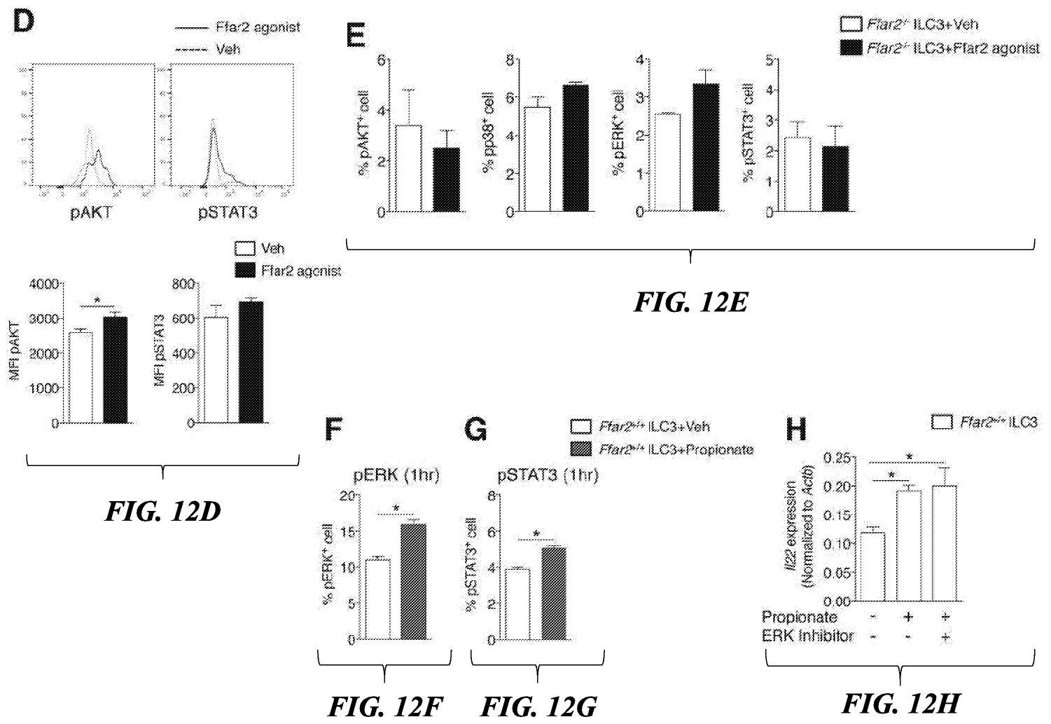

To investigate the signaling downstream of Ffar2 underpinning ILC3-derived IL-22 production, colonic ILC3s were sorted and stimulated the cells ex vivo with acetate, propionate, or the Ffar2 agonist described herein. The Ffar2 agonist and, to a lesser extent, propionate increased IL-22 expression in sorted ILC3s while acetate did not alter IL22 expression (FIG. 11A). The in vitro BrdU incorporation analysis showed that acetate increased the percentage of BrdU+ ILC3s while propionate did not (FIG. 12A), supporting that acetate regulates colonic ILC3 expansion but not ILC3-derived IL-22 production. Ffar2 can couple with Gi/o and/or Gq proteins, and Ffar2 activation can inhibit cyclic AMP (cAMP) and/or stimulate Ca2+ influx, eliciting intracellular signaling that regulates numerous cell-specific functions (Brown et al., 2003; Le Poul et al., 2003; Nilsson et al., 2003).

A Gi/o inhibitor (pertussis toxin [PTX]) and a Gq inhibitor (YM-254890) were employed to determine whether distinctive forms of Ffar2 agonism examined herein differentially affect ILC3-derived IL-22 through direct involvement of Ffar2 downstream canonical signal pathways. Both PTX and YM-254890 abolished IL22 expression in sorted ILC3s stimulated with the synthetic Ffar2 agonist (FIG. 11B). However, these Gi/o and Gq inhibitors, upon activation with propionate, decreased IL22 transcripts to a lesser extent (FIG. 11C). This suggests that the synthetic Ffar2 agonist is likely more efficient in Gi/o and Gq subunit-mediated signaling of Ffar2 compared with propionate and that propionate may engage additional signaling pathways downstream of Ffar2. These findings are consistent with the differential affinities of these agonists for Ffar2. A phosphoprotein expression analysis suggested that Ffar2 agonism with SCFA activated mitogen-activated protein (MAP) kinase pathways, PI3K-AKT, and PKC via Gi/o or Gq proteins in neutrophils (Maslowski et al., 2009).

To further evaluate how Ffar2 signaling regulates colonic ILC3-derived IL-22, protein phosphorylation of candidate Ffar2 downstream effectors were determined by flow cytometry. Ffar2′ ILC3s exhibited reduced percentage and MFI for phosphorylated AKT, p38, and ERK (FIG. 11D and FIG. 12B). Given that STAT3 phosphorylation (pSTAT3) is a critical regulator for ILC3-derived IL-22 (Guo et al., 2014), we examined if Ffar2 signaling regulates STAT3 activation in colonic ILC3s. Ffar2′ ILC3s showed a decreased percentage of pSTAT3+ compared with WT ILC3s (FIG. 11E and FIG. 12C).

To evaluate if Ffar2 agonism activates these signaling molecules in colonic ILC3s, sorted ILC3s were stimulated with propionate or the synthetic Ffar2 agonist ex vivo. The synthetic Ffar2 agonist increased AKT and STAT3 phosphorylation, but not p38 and ERK, in WT ILC3s (FIG. 11F and FIG. 12D). As expected, treatment of Ffar2 ILC3s with the synthetic Ffar2 agonist had no effect on these signaling molecules (FIG. 12E). In contrast, propionate did not induce phosphorylation of these molecules in WT ILC3s (FIG. 11F). However, given that propionate has lower potency for Ffar2 compared with the synthetic Ffar2 agonist and that propionate is likely to induce ERK phosphorylation (pERK) (FIG. 11F), it was determined if a longer treatment with propionate would activate ERK signaling in WT ILC3s. The longer stimulation (1 h versus 30 min) did induce a higher pERK in sorted ILC3s, but the percentage of pERK+ ILC3s was comparable to that observed with the shorter time (FIG. 12F). Under these conditions, it was determined whether the longer time with propionate led to pSTAT3 in ILC3s. Propionate did increase pSTAT3+ ILC3s, but only 1%-1.5% of the ILC3s were pSTAT3+ (FIG. 12G).

Next, Ffar2 agonism-induced signaling pathways were tested to determine whether this pathway directly affected IL-22 expression in sorted ILC3s. ILC3s were pretreated with an AKT or an ERK inhibitor prior to stimulation with the Ffar2 agonist or propionate, respectively. The AKT inhibitor, upon Ffar2 activation with the synthetic Ffar2 agonist, decreased Il22 expression in colonic ILC3s, similar to the STAT3 inhibitor (FIG. 11G). In contrast, the ERK inhibitor, prior to the longer propionate stimulation, did not affect IL-22 expression in sorted ILC3s (FIG. 12H). Also, inhibition of AKT upon Ffar2 activation with the agonist impaired STAT3 activation in ILC3s (FIG. 11H), supporting that AKT activation downstream of Ffar2 may directly affect STAT3 phosphorylation in ILC3s. Collectively, these data support that Ffar2 agonism by the synthetic Ffar2 agonist or propionate differentially regulates colonic ILC3-derived IL-22 expression via AKT and STAT3 axis or partially via ERK and STAT3 activation.

In addition, it was shown that the chemicals, peptides, and recombinant proteins described in TABLE 1, can also be used to modulate Ffar2 activity.

SEQUENCES
(FFAR2 amino acid sequence)
SEQ ID NO: 1
MLPDWKSSLILMAYIIIFLTGLPANLLALRAFVGRIRQPQPAPVHILLLS
LTLADLLLLLLLPFKIIEAASNFRWYLPKVVCALTSFGFYSSIYCSTWLL
AGISIERYLGVAFPVQYKLSRRPLYGVIAALVAWVMSFGHCTIVIIVQYL
NTTEQVRSGNEITCYENFTDNQLDVVLPVRLELCLVLFFIPMAVTIFCYW
RFVWIMLSQPLVGAQRRRRAVGLAVVTLLNFLVCFGPYNVSHLVGYHQRK
SPWWRSIAVVFSSLNASLDPLLFYFSSSVVRRAFGRGLQVLRNQGSSLLG
RRGKDTAEGTNEDRGVGQGEGMPSSDFITE
(FFAR2 gene sequence)
SEQ ID NO: 2
See NCBI Reference Sequence: NC_000019.10

1. A method for treating or preventing a gastrointestinal disease, the method comprising: administering to a subject in need thereof an agent that increases the level or activity of Free Fatty Acid Receptor 2 (Ffar2) in the subject.

2. The method of claim 1, wherein the agent preferentially binds to a Ffar2 receptor.

3. The method of claim 1, wherein the agent induces an increase in the number of group 3 innate lymphoid cells (ILC3s).

4. The method of claim 1, wherein the agent induces secretion of interleukin-22 (IL-22) and/or interleukin-17 (IL-17) from ILC3s.

5. The method of claim 1, wherein the agent is selected from the group consisting of a small molecule, an antibody, a peptide, a genome editing system, a vector, and a nucleic acid.

6. The method of claim 5, wherein the small molecule is a short chain fatty acid (SCFA) pharmaceutically acceptable salt, or derivative thereof.

7. The method of claim 6, wherein the SCFA is selected from the group consisting of: propionic acid, acetic acid, butyric acid, formic acid, isobutyric acid, valeric acid, isovaleric acid, formate, acetate, propionate, butyrate, pentanoate, isobutyrate, valerate, isovalerate, sodium propionate, and pharmaceutically acceptable salts thereof.

8. The method of claim 5, wherein the small molecule is selected from the group consisting of: ES43012-SOD, trans-2-methylcrotonic acid, propiolic acid, angelic acid, compound 34, sodium acetate, sodium propionate, sodium butyrate, formate, pentanoate, (S)-2-(4-chlorophenyl)-3,3-dimethyl-N-(5-phenylthiazol-2-yl)butanamide, BTI-A-404, BTI-A-292, AZ1729, and any derivative thereof.

9. The method of claim 5, wherein the vector or nucleic acid encodes an Ffar2 polypeptide.

10.-13. (canceled)

14. The method of claim 1, wherein the agent is formulated with a pharmaceutical composition.

15. The method of claim 14, wherein the pharmaceutical composition is formulated to restrict delivery of the agent to the gastrointestinal tract of the subject.

16. (canceled)

17. The method of claim 1, wherein the gastrointestinal disease is selected from the group consisting of: a gastrointestinal infection, inflammatory bowel disease (IBD), gastrointestinal injury, appendicitis, Crohn's disease (CD), ulcerative colitis (UC), gastritis, enteritis, esophagitis, gastroesophageal reflux disease (GERD), celiac disease, diverticulitis, food intolerance, ulcer, infectious colitis, irritable bowel syndrome, and cancer.

18. The method of claim 1, wherein the administering reduces inflammation of the gastrointestinal tract.

19.-23. (canceled)

24. A method of reducing inflammation in the gastrointestinal tract of a subject, the method comprising: administering to a subject an agent that increases the level or activity of Free Fatty Acid Receptor 2 (Ffar2) in the subject.

25. The method of claim 24, wherein the agent preferentially binds to a Ffar2 receptor.

26. The method of claim 24, wherein the agent induces an increase in the number of group 3 innate lymphoid cells (ILC3s).

27. The method of claim 24, wherein the agent induces secretion of interleukin-22 (IL-22) and/or interleukin-17 (IL-17) from ILC3s.

28. The method of claim 24, wherein the agent is selected from the group consisting of a small molecule, an antibody, a peptide, a genome editing system, a vector, and a nucleic acid.

29. The method of claim 28, wherein the small molecule is a short chain fatty acid (SCFA) pharmaceutically acceptable salt, or derivative thereof.

30.-35. (canceled)

36. An assay for identifying an agent that modulates a functional property of an immune lymphoid cell, the assay comprising:
a. contacting a population of innate lymphoid cells with an agent; and
b. detecting the level of Ffar2
wherein detecting a change in Ffar2 levels after contacting step (a) identifies the agent as one that can modulate a functional property of innate lymphoid cells.

37.-52. (canceled)
